(19) United States
(12) Patent Application Publication
(10) Pub. No.: US 2021/0369842 A1
(43) Pub. Date: Dec. 2, 2021

(54) ANTIBODY FORMULATION

(71) Applicants: GENMAB A/S, Copenhagen V (DK); BIONTECH SE, Mainz (DE)

(72) Inventors: Isil ALTINTAS, Utrecht (NL); David SATIJN, Utrecht (NL); Rik RADEMAKER, Utrecht (NL); Paul PARREN, Odijk (NL); Ugur SAHIN, Mainz (DE); Friederike GIESEKE, Mainz (DE); Alexander MUIK, Mainz (DE)

(21) Appl. No.: 17/289,602

(22) PCT Filed: Nov. 6, 2019
(86) PCT No.: PCT/EP2019/080445

§ 371 Date: Apr. 28, 2021

(30) Foreign Application Priority Data
Nov. 6, 2018 (EP) PCT/EP2018/080369

(51) Int. Cl. A61K 39/395; C07K 16/28; A61P 35/00; A61K 47/22; A61K 47/26; A61K 9/00

(52) U.S. Cl. CPC A61K 39/39591; C07K 16/2878; C07K 16/2827; A61K 2039/505; A61K 47/22; A61K 47/26; A61K 9/0019; A61P 35/00

(57) ABSTRACT

The present invention relates to pharmaceutical formulations of binding agents and their use in medicine. In particular, the invention relates to pharmaceutical formulations of binding agents such as bispecific antibodies binding human PD-L1 and binding human CD137. The invention furthermore relates to uses of the pharmaceutical formulations of the invention and to methods for producing pharmaceutical formulations.

### FIELD OF INVENTION

The present invention relates to bispecific antibodies binding to PD-L1 and CD137 (4-1BB). The invention provides pharmaceutical compositions comprising the antibodies and use of the formulations for therapeutic.

### BACKGROUND

CD137 (4-1BB, TNFRSF9) is a member of the tumor necrosis factor (TNF) receptor (TNFR) family. CD137 is a co-stimulatory molecule on CD8⁺ and CD4⁺ T cells, regulatory T cells (Tregs), natural killer (NK) and NKT cells, B cells and neutrophils. On T cells, CD137 is not constitutively expressed, but induced upon T-cell receptor (TCR)-activation. Stimulation via its natural ligand 4-1BBL or agonist antibodies leads to signaling using TNFR-associated factor (TRAF)-2 and TRAF-1 as adaptors. Early signaling by CD137 involves K-63 poly-ubiquitination reactions that ultimately result in activation of the nuclear factor (NF)-κB and mitogen-activated protein (MAP)-kinase pathways. Signaling leads to increased T cell co-stimulation, proliferation, cytokine production, maturation and prolonged CD⁸⁺ T-cell survival. Agonistic antibodies against CD137 have been shown to promote anti-tumor control by T cells in various pre-clinical models (Murillo et al. 2008 Clin. Cancer Res. 14(21): 6895-6906). Antibodies stimulating CD137 can induce survival and proliferation of T cells, thereby enhancing the anti-tumor immune response. Antibodies stimulating CD137 have been disclosed in the prior art, and include urelumab, a human IgG4 antibody (WO2005035584) and utomilumab, a human IgG2 antibody (Fisher et al. 2012 Cancer Immunol. Immunother. 61: 1721-1733).

Programmed death ligand 1 (PD-L1, PDL1, CD274, B7H1) is a 33 kDa, single-pass type I membrane protein. Three isoforms of PD-L1 have been described, based on alternative splicing. PD-L1 belongs to the immunoglobulin (Ig) superfamily and contains one Ig-like C2-type domain and one Ig-like V-type domain. Freshly isolated T and B cells express negligible amounts of PD-L1 and a fraction (about 16%) of CD14⁺ monocytes constitutively express PD-L1. However, interferon-γ (IFNγ) is known to upregulate PD-L1 on tumor cells.

PD-L1 obstructs anti-tumor immunity by 1) tolerizing tumor-reactive T cells by binding to its receptor, programmed cell death protein 1 (PD-1) (CD279) on activated T cells; 2) rendering tumor cells resistant to CD8⁺ T cell and Fas ligand-mediated lysis by PD-1 signaling through tumor cell-expressed PD-L1; 3) tolerizing T cells by reverse signaling through T cell-expressed CD80 (B7.1); and 4) promoting the development and maintenance of induced T regulatory cells. PD-L1 is expressed in many human cancers, including melanoma, ovarian, lung and colon cancer (Latchman et al., 2004 Proc Natl Acad Sci USA 101, 10691-6). PD-L1 blocking antibodies have shown clinical activity in several cancers known to overexpress PD-L1 (incl. melanoma, NSCLC). For example, atezolizumab is a humanized IgG1 monoclonal antibody against PD-L1. It is currently in clinical trials as an immunotherapy for several indications including various types of solid tumors (see e.g. Rittmeyer et al., 2017 Lancet 389:255-265) and is approved for non-small-cell lung cancer and bladder cancer indications. Avelumab, a PD-L1 antibody, (Kaufman et al Lancet Oncol. 2016; 17(10):1374-1385) has been approved by the FDA for the treatment of adults and pediatric patients 12 years and older with metastatic Merkel cell carcinoma, and is currently in clinical trials in several cancer indications, including bladder cancer, gastric cancer, head and neck cancer, mesothelioma, NSCLC, ovarian cancer and renal cancer. Durvalumab, a PD-L1 antibody, is approved for locally advanced or metastatic urothelial carcinoma indications and is in clinical development in multiple solid tumors and blood cancers (see e.g. Massard et al., 2016 J Clin Oncol. 34(26):3119-25). Further anti-PD-L1 antibodies have been described in WO2004004771, WO2007005874, WO2010036959, WO2010077634, WO2013079174, WO2013164694, WO2013173223 and WO2014022758.

Horton et al (J Immunother Cancer. 2015; 3(Suppl 2): 010) discloses combination of an agonistic 4-1BB antibody with a neutralizing PD-L1 antibody.

Combination therapy of utomilumab and avelumab is currently being tested in the clinic (Chen et al., J Clin Oncol 35, 2017 suppl; abstr TPS7575, and clinical trial NCT02554812).

However, despite advances in the art, there is a need for multispecific antibodies that can bind both PD-L1 and CD137 and pharmaceutical formulations of the same.

### SUMMARY OF INVENTION

It is an object of the present invention to provide a pharmaceutical formulation comprising
a. a binding agent comprising a first antigen-binding region binding to human CD137 (4-1BB) and a second antigen-binding region binding to human PD-L1 (CD274),
   the first antigen biding region comprising a first heavy chain variable region (VH) comprising the three complementarity determining regions, CDR1, CDR2, and CDR3, present within the amino acid sequence set forth in SEQ ID NO: 15, and a first light chain variable region (VL) comprising the three complementarity determining regions, CDR1, CDR2, and CDR3, present within the amino acid sequence set forth in SEQ ID NO: 16, and
   the second antigen-binding region comprising a second heavy chain variable region (VH) comprising the three complementarity determining regions, CDR1, CDR2, and CDR3, present within the amino acid sequence set forth in SEQ ID NO: 17, and a second light chain variable region (VL) comprising the three complementarity determining regions, CDR1, CDR2, and CDR3, present within the amino acid sequence set forth in SEQ ID NO: 21;
b. a histidine buffer,
c. about 100 to about 400 mM of a sugar, and
d. about 0.001 to about 0.1% (w/v) non-ionic surfactant;
   and having a pH between about 4.5 and about 6.5.

In another aspect, the present invention relates to a pharmaceutical formulation as defined above for use as a medicament.

In another aspect, the present invention relates to a pharmaceutical formulation as defined above for use in the treatment of cancer.

In yet another aspect, the present invention relates to a method of treatment of a disease comprising administering an effective amount of a pharmaceutical formulation as defined above to a subject in need thereof

In still another aspect, the present invention relates to a method of inducing cell death, or inhibiting growth and/or proliferation of a tumor cell expressing PD-L1 comprising administering an effective amount of a pharmaceutical formulation as defined above to a subject in need thereof and/or bearing said tumor cell.

It is also within the scope of the invention to provide the use of the pharmaceutical formulation defined above for the manufacture of a medicament, such as a medicament for the treatment of cancer, e.g. a cancer characterized by the presence of solid tumors or a cancer selected from the group consisting of: melanoma, ovarian cancer, lung cancer, colon cancer and head and neck cancer.

Finally the invention provides a method for producing a pharmaceutical formulation of the invention, the method comprising providing a binding agent as defined herein and combining it with:
a. a histidine buffer,
b. about 100 to about 400 mM of a sugar, and
c. about 0.001 to about 0.1% (w/v) non-ionic surfactant;
   at a pH between about 4.5 and about 6.5.

### BRIEF DESCRIPTION OF THE DRAWINGS

FIG. 1: Sequence alignments for human, African elephant and wild boar CD137. Amino acids in African elephant and wild boar CD137 that differ from those in the human sequence are highlighted in black.

FIG. 2: CD137 shuffle constructs, containing African elephant (shuffle 5) or wild boar (shuffle 1-4, 6) CD137 domains.

FIG. 3: Expression of CD137 shuffle constructs on HEK293-T17 cells. HEK293-T17 cells were transfected with the CD137 shuffle constructs. Cell surface expression of the constructs was measured by flow cytometry, using a polyclonal anti-CD137 antibody that recognizes human, wild boar and African elephant CD137.

FIG. 4: Binding of antibody CD137-009 to CD137 shuffle constructs expressed on HEK293-T17 cells. HEK293-T17 cells were transfected with the CD137 shuffle constructs, and with human CD137 (hCD137 wt), African elephant of wild boar CD137. Binding of antibody CD137-009 to these constructs expressed on HEK293-T17 cells was measured by flow cytometry. Staining with polyclonal anti-CD137 antibody is shown as a control.

FIG. 5: Effect of monovalent antibody b12-FEALxPD-L1-547-FEAR on the PD-1/PD-L1 interaction. The effect of b12-FEALxPD-L1-547-FEAR was determined in a PD-1/PD-L1 inhibition bioassay. Data shown are fold induction relative to control (without antibody added), for one representative experiment.

FIG. 6: Schematic representation of the anticipated mode of action of CD137xPD-L1 bispecific antibodies. (A) PD-L1 is expressed on antigen-presenting cells (APCs) as well as on tumor cells. PD-L1 binding to T cells expressing the negative regulatory molecule PD-1 effectively overrides T cell activation signals and eventually leads to T cell inhibition. (B) Upon addition of a CD137xPD-L1 bispecific antibody, the inhibitory PD-1:PD-L1 interaction is blocked via the PD-L1-specific arm and at the same time, the bispecific antibody, through the cell-cell interaction provides agonistic signaling to CD137 expressed on the T cells resulting in strong T cell costimulation.

FIG. 7: Release of the PD-1/PD-L1-mediated T cell inhibition and additional co-stimulation of CD⁸⁺ T cell proliferation by CD137-009-FEALxPD-L1-547-FEAR in an antigen-specific T cell assay with active PD-1/PD-L1 axis. CFSE-labelled T cells electroporated with a claudin-6-specific TCR- and PD-1-in vitro translated (IVT)-RNA were incubated with claudin-6-IVT-RNA-electroporated immature dendritic cells in the presence of 0.1 μg/mL and 0.02 μg/mL CD137-009-FEALxPD-L1-547-FEAR, b12-FEALxPD-L1-547-FEAR or b12 control antibody for five days. CD⁸⁺ T cell proliferation was measured by flow cytometry. Data shown are (A and C) representative CFSE histogram from two different donors and (B and D) the corresponding percentages of divided cells and proliferation index as calculated using FlowJo software. (B) shows analysis of data from donor 1 representatively shown in (A). (D) shows analysis of data from donor 2 representatively shown in (C). Error bars (SD) indicate variation within the experiment (three replicates, using cells from one donor).

FIG. 8: Analysis of the EC₅₀ value of the bispecific antibody CD137-009-FEALxPD-L1-547-FEAR in an antigen-specific T cell assay with active PD1/PD-L1 axis. CFSE-labeled T cells electroporated with a claudin-6-specific TCR- and PD-1-IVT-RNA were incubated with claudin-6-IVT-RNA-electroporated immature dendritic cells in the presence of CD137-009-FEALxPD-L1-547-FEAR (at 3-fold serial dilutions from 1 to 0.00015 μg/mL) for five days. CD8⁺ T cell proliferation was measured by flow cytometry. Data shown are percentages of divided cells (open diamonds) and proliferation indices (filled triangles) as a function of the antibody concentration. Error bars (SD) indicate variation within the experiment (six replicates, using cells from one donor). Curves were fitted by nonlinear regression and ECso values were determined using GraphPad Prism software.

FIG. 9: Comparison of CD137-009-FEALxPD-L1-547-FEAR with a combination of the two monovalently binding CD137 antibodies (CD137-009-FEALxb12-FEAR+b12-FEALxPD-L1-547-FEAR) or the two parental antibodies (CD137-009+PD-L1-547) in an antigen-specific T cell assay with active PD1/PD-L1 axis. CFSE-labelled T cells electroporated with a claudin-6-specific TCR- and PD1-IVT-RNA were incubated with claudin-6-IVT-RNA electroporated immature dendritic cells in the presence of 0.25 μg/mL (i) CD137-009-FEALxPD-L1-547-FEAR, (ii) CD137-009-FEALxb12+b12-FEALxPD-L1-547-FEAR, (iii) CD137-009-FEALxb12, (iv) b12-FEALxPD-L1-547-FEAR, (v) CD137-009+PD-L1-547, (vi) CD137-009, (vii) PD-L1-547, or (viii) b12 control antibody for five days. CD⁸⁺ T cell proliferation was measured by flow cytometry. Data shown are (A) representative CFSE histograms and (B and C) the corresponding mean values of percent divided cells and proliferation index as calculated using FlowJo software. Error bars (SD) indicate the variation within the experiment (three replicates, using cells from one donor)

FIG. 10: Ex vivo expansion of tumor infiltrating lymphocytes (TIL) from a human non-small-cell lung cancer tissue resection by CD137-009-FEALxPD-L1-547-FEAR. Tumor pieces from the resected tissue were cultured with 10 U/mL IL-2 and the indicated concentration of CD137-009-FEALxPD-L1-547-FEAR. After 10 days of culture, cells were harvested and analyzed by flow cytometry. (A) TIL count as fold expansion compared to untreated controls, (B) CD3⁺ CD8⁺ T cell count as fold expansion compared to untreated controls, (C) CD3⁺ CD4⁺ T cell count as fold expansion compared to untreated controls, (D) CD3⁻CD56⁺ NK cell count as fold expansion compared to untreated controls. Bars represent the mean±SD of n=5 individual wells, with two tumor pieces per well as starting material.

FIG. 11: Effect of mCD137-3H3xmPD-L1-MPDL3280A mouse surrogate antibody on antigen-specific T cell proliferation in an OT-I adoptive cell transfer set up. Ovalbumin (OVA) specific OT1⁺Thy1.1⁺ double positive cytotoxic T cells isolated from donor mice were retro-orbitally (r.o.) injected into naïve C57BL/6 recipient mice. The day after adoptive cell transfer, recipient mice were injected r.o. with 100 μg OVA as antigenic stimulus followed by a r.o. injection of 100 μg or 20 μg mCD137-3H3xmPD-L1-MPDL3280A, mCD137-3H3xb12 or mPD-L1-MPDL3280Axb12 antibody per mouse. Injection of PBS (indicated as OVA alone in the figure) was used as baseline reference and untreated animals were used as negative control. After 6 days, 100 μL blood was drawn via the r.o. route and analyzed for Thy1.1+CD⁸⁺ T cells. Data shown are (A) a schematic representation of the OT-I adoptive cell transfer experimental outline, and (B) the Thy1.1+CD⁸⁺ T cell frequency for each treatment group at day 6. Squares represent individual animals and error bars (SD) indicate the variation within the experiment (n=5 mice per group). Statistical analysis was performed using One-way Anova with Tukey's multiple comparisons test; ns=no significant difference between groups, ***=P<0.001.

FIG. 12: Anti-tumor efficacy of the mCD137-3H3xmPD-L1-MPDL3280A mouse surrogate antibody in a subcutaneous, syngeneic CT26 mouse tumor model. Female BALB/c mice bearing subcutaneous CT26 tumors were treated with intraperitoneal injections of 20 μg (i) mCD137-3H3xmPD-L1-MPDL3280A, (ii) mCD137-3H3xb12 or (iii) mPD-L1-MPDL3280Axb12 antibody per mouse, or (iv) PBS, after tumors reached a volume 30 mm³. Dosing schedule was: every 2-3 days for the first eight injections, followed by an injection every 7 days until the end of the experiment. At day 29, 100 μL blood was drawn via the r.o. route and analyzed for gp70-specific CD8⁺ T cells. Data shown are (A) tumor growth curves with each line representing a single mouse, (B) the resulting Kaplan-Meier survival analysis, and (C) the gp70-specific CD8⁺ T-cell frequencies for each treatment group at day 29 post implantation. PFS=progression free survival.

FIG. 13: Binding of monospecific, bivalent PD-L1 antibodies and monovalent b12xPD-L1 antibodies to tumor cells. Binding of PD-L1-547 and b12-FEALxPD-L1-547-FEAR to MDA-MB-231 (A), PC-3 (B) and SK-MES-1 (C) cells. Data shown are mean fluorescence intensities (MFI) as determined by flow cytometry. Monospecific, bivalent b12 antibodies were included as negative control.

FIG. 14: Comparison of PD-L1-547-FEALxCD137-009-HC7LC2-FEAR with a combination of the two monovalent controls (b12-FEALxCD137-009-HC7LC2-FEAR+b12-FEALxPD-L1-547-FEAR) or the two parental antibodies (CD137-009-HC7LC2-FEAR+PD-L1-547-FEAR) in a non-antigen-specific T-cell proliferation assay. CFSE-labeled PBMCs were incubated with sub-optimal concentration of anti-CD3 antibody (0.03 μg/mL and 0.1 μg/mL), or without (w/o) anti-CD3 antibody (as negative control for T-cell activation), and cultured in the presence of 0.2 μg/mL i) PD-L1-547-FEALxCD137-009-HC7LC2-FEAR, ii) b12-FEALxCD137-009-HC7LC2-FEAR+b12-FEALxPD-L1-547-FEAR each, iii) b12-FEALxCD137-009-HC7LC2-FEAR, iv) b12-FEALxPD-L1-547-FEAR, v) CD137-009-HC7LC2-FEAR+PD-L1-547-FEAR each, vi) CD137-009-HC7LC2-FEAR, vii) PD-L1-547-FEAR, or viii) b12-IgG-FEAL control antibody for four days. CD⁴⁺ (A) and CD8+(B) T-cell proliferation was measured by flow cytometry. Data are shown from three donors as the mean expansion index of three replicates, as calculated using FlowJo v10.4 software. Error bars (SD) indicate the variation within the experiment (three replicates, using cells from one donor).

FIG. 15: Determination of EC₅₀ values for induction of T-cell proliferation by PD-L1-547-FEALxCD137-009-HC7LC2-FEARx in a non-antigen-specific T-cell proliferation assay. CFSE-labeled PBMCs were incubated for four days with a sub-optimal concentration of anti-CD3 antibody and serial dilutions of PD-L1-547-FEALxCD137-009-HC7LC2-FEAR (1-0.00015 μg/mL) or 1 μg/mL b12 IgG as control antibody. Data from two representative donors are shown; PBMCs from donor 1 were stimulated with 0.03 μg/mL anti-CD3 (A, B) and PBMCs from donor 2 with 0.09 μg/mL anti-CD3 (C, D). CD⁴⁺ (A and C) and CD8+(B and D) T-cell proliferation was measured by flow cytometry. Data shown are mean expansion index values of three replicates, as calculated using FlowJo v10.4 software and fitted with a four parameter logarithmic fit. Error bars (SD) indicate the variation within the experiment (three replicates, using cells from one donor).

FIG. 16: Effect of PD-L1-547-FEALxCD137-009-HC7LC2-FEAR on secretion of 10 pro-inflammatory cytokines in an antigen-specific T-cell assay with or without PD-1 electroporation into T cells. T cells electroporated with a CLDN6-specific TCR- and with or without 2 μg PD1-IVT-RNA were incubated with CLDN6-IVT-RNA-electroporated iDCs in the presence of different concentrations of CD137-009-HC7LC2-FEALxPD-L1-547-FEAR (three-fold serial dilutions; ranging from 1 μg/mL to 0.00015 μg/mL) or b12 control antibody b12-IgG-FEAL. Cytokine levels of supernatants were determined 48 hours after antibody addition by multiplex sandwich immunoassay using the MSD V-Plex Human Proinflammatory panel 1 (10-Plex) kit. Each data point represents mean±SD of three individual wells.

FIG. 17: Effect of PD-L1-547-FEALxCD137-009-HC7LC2-FEAR on secretion of 10 pro-inflammatory cytokines in an antigen-unspecific T-cell assay. Human PBMCs were sub-optimally stimulated with anti-CD3 antibody in the presence of different concentrations of PD-L1-547-FEALxCD137-009-HC7LC2-FEAR (three-fold serial dilutions; ranging from 1 μg/mL to 0.00015 μg/mL) or b12 control antibody b12-IgG-FEAL. Cytokine levels in supernatants were determined at 48 hours after antibody addition by multiplex sandwich immunoassay using the MSD V-Plex Human Proinflammatory panel 1 (10-Plex) kit. Each data point represents mean±SD of three individual wells.

### DETAILED DESCRIPTION

### Definitions

The term “binding agent” in the context of the present invention refers to any agent capable of binding to desired antigens. In certain embodiments of the invention, the binding agent is an antibody, antibody fragment, or construct thereof. The binding agent may also comprise synthetic, modified or non-naturally occurring moieties, in particular non-peptide moieties. Such moieties may, for example, link desired antigen-binding functionalities or regions such as antibodies or antibody fragments. In one embodiment, the binding agent is a synthetic construct comprising antigen-binding CDRs or variable regions.

The term “immunoglobulin” refers to a class of structurally related glycoproteins consisting of two pairs of polypeptide chains, one pair of light (L) low molecular weight chains and one pair of heavy (H) chains, all four inter-connected by disulfide bonds. The structure of immunoglobulins has been well characterized. See for instance Fundamental Immunology Ch. 7 (Paul, W., ed., 2nd ed. Raven Press, N.Y. (1989)). Briefly, each heavy chain typically is comprised of a heavy chain variable region (abbreviated herein as V_{H} or V_{H}) and a heavy chain constant region (abbreviated herein as C_{H} or C_{H}). The heavy chain constant region typically is comprised of three domains, CH1, CH2, and CH3. The hinge region is the region between the CH1 and CH2 domains of the heavy chain and is highly flexible. Disulphide bonds in the hinge region are part of the interactions between two heavy chains in an IgG molecule. Each light chain typically is comprised of a light chain variable region (abbreviated herein as V_{L} or VL) and a light chain constant region (abbreviated herein as C_{L} or CL). The light chain constant region typically is comprised of one domain, CL. The V_{H} and VL regions may be further subdivided into regions of hypervariability (or hypervariable regions which may be hypervariable in sequence and/or form of structurally defined loops), also termed complementarity determining regions (CDRs), interspersed with regions that are more conserved, termed framework regions (FRs). Each VH and VL is typically composed of three CDRs and four FRs, arranged from amino-terminus to carboxy-terminus in the following order: FR1, CDR1, FR2, CDR2, FR3, CDR3, FR4 (see also Chothia and Lesk J. Mol. Biol. 196, 901-917 (1987)). Unless otherwise stated or contradicted by context, CDR sequences herein are identified according to IMGT rules using DomainGapAlign (Lefranc M P., Nucleic Acids Research 1999; 27:209-212 and Ehrenmann F., Kaas Q. and Lefranc M.-P. Nucleic Acids Res., 38, D301-307 (2010); see also internet http address www.imgt.org/). Unless otherwise stated or contradicted by context, reference to amino acid positions in the constant regions in the present invention is according to the EU-numbering (Edelman et al., Proc Natl Acad Sci USA. 1969 May; 63(1):78-85; Kabat et al., Sequences of Proteins of Immunological Interest, Fifth Edition. 1991 NIH Publication No. 91-3242).

The term “isotype” as used herein refers to the immunoglobulin class (for instance IgG1, IgG2, IgG3, IgG4, IgD, IgA, IgE, or IgM) or any allotypes thereof, such as IgG1m(za) and IgG1m(f)) that is encoded by heavy chain constant region genes. Further, each heavy chain isotype can be combined with either a kappa (κ) or lambda (λ) light chain.

The term “antibody” (Ab) in the context of the present invention refers to an immunoglobulin molecule, a fragment of an immunoglobulin molecule, or a derivative of either thereof, which has the ability to specifically bind to an antigen under typical physiological conditions with a half-life of significant periods of time, such as at least about 30 minutes, at least about 45 minutes, at least about one hour, at least about two hours, at least about four hours, at least about 8 hours, at least about 12 hours, about 24 hours or more, about 48 hours or more, about 3, 4, 5, 6, 7 or more days, etc., or any other relevant functionally-defined period (such as a time sufficient to induce, promote, enhance, and/or modulate a physiological response associated with antibody binding to the antigen and/or time sufficient for the antibody to recruit an effector activity). The variable regions of the heavy and light chains of the immunoglobulin molecule contain a binding domain that interacts with an antigen. The term “antigen-binding region”, wherein used herein, refers to the region which interacts with the antigen and comprises both a VH region and a VL region. The term antibody when used herein comprises not only monospecific antibodies, but also multispecific antibodies which comprise multiple, such as two or more, e.g. three or more, different antigen-binding regions. The constant regions of the antibodies (Abs) may mediate the binding of the immunoglobulin to host tissues or factors, including various cells of the immune system (such as effector cells) and components of the complement system such as C1q, the first component in the classical pathway of complement activation. As indicated above, the term antibody herein, unless otherwise stated or clearly contradicted by context, includes fragments of an antibody that are antigen-binding fragments, i.e., retain the ability to specifically bind to the antigen. It has been shown that the antigen-binding function of an antibody may be performed by fragments of a full-length antibody. Examples of antigen-binding fragments encompassed within the term “antibody” include (i) a Fab′ or Fab fragment, a monovalent fragment consisting of the VL, VH, CL and CH1 domains, or a monovalent antibody as described in WO2007059782 (Genmab); (ii) F(ab′)2 fragments, bivalent fragments comprising two Fab fragments linked by a disulfide bridge at the hinge region; (iii) a Fd fragment consisting essentially of the VH and CH1 domains; (iv) a Fv fragment consisting essentially of the VL and VH domains of a single arm of an antibody, (v) a dAb fragment (Ward et al., Nature 341, 544-546 (1989)), which consists essentially of a VH domain and also called domain antibodies (Holt et al; Trends Biotechnol. 2003 November; 21(11):484-90); (vi) camelid or Nanobody molecules (Revets et al; Expert Opin Biol Ther. 2005 January; 5(1):111-24) and (vii) an isolated complementarity determining region (CDR). Furthermore, although the two domains of the Fv fragment, VL and VH, are coded for by separate genes, they may be joined, using recombinant methods, by a synthetic linker that enables them to be made as a single protein chain in which the VL and VH regions pair to form monovalent molecules (known as single chain antibodies or single chain Fv (scFv), see for instance Bird et al., Science 242, 423-426 (1988) and Huston et al., PNAS USA 85, 5879-5883 (1988)). Such single chain antibodies are encompassed within the term antibody unless otherwise noted or clearly indicated by context. Although such fragments are generally included within the meaning of antibody, they collectively and each independently are unique features of the present invention, exhibiting different biological properties and utility. These and other useful antibody fragments in the context of the present invention, as well as bispecific formats of such fragments, are discussed further herein. It also should be understood that the term antibody, unless specified otherwise, also includes polyclonal antibodies, monoclonal antibodies (mAbs), antibody-like polypeptides, such as chimeric antibodies and humanized antibodies, and antibody fragments retaining the ability to specifically bind to the antigen (antigen-binding fragments) provided by any known technique, such as enzymatic cleavage, peptide synthesis, and recombinant techniques. An antibody as generated can possess any isotype. As used herein, the term “isotype” refers to the immunoglobulin class (for instance IgG1, IgG2, IgG3, IgG4, IgD, IgA, IgE, or IgM) that is encoded by heavy chain constant region genes. When a particular isotype, e.g. IgG1, is mentioned herein, the term is not limited to a specific isotype sequence, e.g. a particular IgG1 sequence, but is used to indicate that the antibody is closer in sequence to that isotype, e.g. IgG1, than to other isotypes. Thus, e.g. an IgG1 antibody of the invention may be a sequence variant of a naturally-occurring IgG1 antibody, including variations in the constant regions.

When used herein, the terms “arm”, “Fab-arm” and “half molecule” refer to one heavy chain-light chain pair. When a bispecific antibody is described to comprise a half-molecule antibody “derived from” a first antibody, and a half-molecule antibody “derived from” a second antibody, the term “derived from” indicates that the bispecific antibody was generated by recombining, by any known method, said half-molecules from each of said first and second antibodies into the resulting bispecific antibody. In this context, “recombining” is not intended to be limited by any particular method of recombining and thus includes all of the methods for producing bispecific antibodies described herein below, including for example recombining by half-molecule exchange, as well as recombining at nucleic acid level and/or through co-expression of two half-molecules in the same cells.

The term “antigen-binding region” or “binding region” as used herein, refers to a region of an antibody which is capable of binding to the antigen. The antigen can be any molecule, such as a polypeptide, e.g. present on a cell, bacterium, or virion. The terms “antigen-binding region” and “antigen-binding site” may, unless contradicted by the context, be used interchangeably in the context of the present invention.

The terms “antigen” and “target” may, unless contradicted by the context, be used interchangeably in the context of the present invention.

The term “binding” as used herein refers to the binding of an antibody to a predetermined antigen or target, typically with a binding affinity corresponding to a K_{D} of 1E⁻⁶ M or less, e.g. 5E⁻⁷ M or less, 1E⁻⁷ M or less, such as 5E⁻⁸ M or less, such as 1E⁻⁸ M or less, such as 5E⁻⁹ M or less, or such as 1E⁻⁹ M or less, when determined by biolayer interferometry using the antibody as the ligand and the antigen as the analyte and binds to the predetermined antigen with an affinity corresponding to a K_{D} that is at least ten-fold lower, such as at least 100-fold lower, for instance at least 1,000-fold lower, such as at least 10,000-fold lower, for instance at least 100,000-fold lower than its affinity for binding to a non-specific antigen (e.g., BSA, casein) other than the predetermined antigen or a closely-related antigen.

The term “K_{D}” (M), as used herein, refers to the dissociation equilibrium constant of a particular antibody-antigen interaction, and is obtained by dividing k_{d} by kₐ.

The term “k_{d}” (sec⁻¹), as used herein, refers to the dissociation rate constant of a particular antibody-antigen interaction. Said value is also referred to as the koff value or off-rate.

The term “kₐ” (M⁻¹×sec⁻¹), as used herein, refers to the association rate constant of a particular antibody-antigen interaction. Said value is also referred to as the kₒₙ value or on-rate.

The term “PD-L1” when used herein, refers to the Programmed Death-Ligand 1 protein. PD-L1 is found in humans and other species, and thus, the term “PD-L1” is not limited to human PD-L1 unless contradicted by context. Human, macaque (cynomolgus monkey), African elephant, wild boar and mouse PD-L1 sequences can be found through Genbank accession no. NP_054862.1, XP_005581836, XP_003413533, XP_005665023 and NP_068693, respectively. The sequence of human PD-L1 is also shown in SEQ ID NO: 28, wherein amino acids 1-18 are predicted to be a signal peptide. The sequence of macaque (cynomolgus monkey) PD-L1 is also shown in SEQ ID NO: 29, wherein amino acids 1-18 are predicted to be a signal peptide.

The term “CD137” as used herein, refers to the human Cluster of Differentiation 137 protein. CD137 (4-1BB), also referred to as TNFRSF9, is the receptor for the ligand TNFSF9/4-1BBL. CD137 is believed to be involved in T cell activation. In one embodiment, CD137 is human CD137, having UniProt accession number Q07011. The sequence of human CD137 is also shown in SEQ ID NO: 30, wherein amino acids 1-23 are predicted to be a signal peptide. In one embodiment CD137 is cynomolgus monkey (Macaca fascicularis) CD137, having UniProt accession number A9YYE7-1. The sequence of cynomolgus monkey CD137 is shown in SEQ ID NO: 31, wherein amino acids 1-23 are predicted to be aa signal peptide. Wild boar (Sus scrofa) CD137 is shown in SEQ ID NO: 38, wherein amino acids 1-23 are predicted to be aa signal peptide. African elefant (Loxodonta africana) CD137 is shown in SEQ ID NO: 39, wherein amino acids 1-23 are predicted to be aa signal peptide.

A “PD-L1 antibody” or “anti-PD-L1 antibody” is an antibody as described above, which binds specifically to the antigen PD-L1, in particular human PD-L1.

A “CD137 antibody” or “anti-CD137 antibody” is an antibody as described above, which binds specifically to the antigen CD137.

A “CD137xPD-L1 antibody”, “anti-CD137xPD-L1 antibody”, “PD-L1xCD137 antibody” or “anti-PD-L1xCD137 antibody” is a bispecific antibody, which comprises two different antigen-binding regions, one of which binds specifically to the antigen PD-L1 and one of which binds specifically to CD137.

The term “bispecific antibody” refers to antibody having specificities for at least two different, typically non-overlapping, epitopes. Such epitopes may be on the same or different targets. For the present invention the epitopes are on the same target, namely PD-L1 and 4-1BB. Examples of different classes of bispecific antibodies comprising an Fc region include but are not limited to: asymmetric bispecific molecules, e.g., IgG-like molecules with complementary CH3 domains; and symmetric bispecific molecules, e.g., recombinant IgG-like dual targeting molecules wherein each antigen-binding region of the molecule binds at least two different epitopes.

Examples of bispecific molecules include but are not limited to Triomab® (Trion Pharma/Fresenius Biotech, WO/2002/020039), Knobs-into-Holes (Genentech, WO 1998/50431), CrossMAbs (Roche, WO 2009/080251, WO 2009/080252, WO 2009/080253), electrostatically-matched Fc-heterodimeric molecules (Amgen, EP1870459 and WO2009089004; Chugai, US201000155133; Oncomed, WO 2010/129304), LUZ-Y (Genentech), DIG-body, PIG-body and TIG-body (Pharmabcine), Strand Exchange Engineered Domain body (SEEDbody) (EMD Serono, WO2007110205), Bispecific IgG1 and IgG2 (Pfizer/Rinat, WO 2011/143545), Azymetric scaffold (Zymeworks/Merck, WO2012058768), mAb-Fv (Xencor, WO 2011/028952), XmAb (Xencor), Bivalent bispecific antibodies (Roche, WO 2009/080254), Bispecific IgG (Eli Lilly), DuoBody® molecules (Genmab A/S, WO 2011/131746), DuetMab (Medimmune, US2014/0348839), Biclonics (Merus, WO 2013/157953), NovImmune (KABodies, WO 2012/023053), FcAAdp (Regeneron, WO 2010/151792), (DT)-Ig (GSK/Domantis), Two-in-one Antibody or Dual Action Fabs (Genentech, Adimab), mAb2 (F-Star, WO 2008/003116), Zybody™ molecules (Zyngenia), CovX-body (CovX/Pfizer), FynomAbs (Covagen/Janssen Cilag), DutaMab (Dutalys/Roche), iMab (MedImmune), Dual Variable Domain (DVD)-Ig™ (Abbott), dual domain double head antibodies (Unilever; Sanofi Aventis, WO 2010/0226923), Ts2Ab (MedImmune/AZ), BsAb (Zymogenetics), HERCULES (Biogen Idec, U.S. Pat. No. 7,951,918), scFv-fusions (Genentech/Roche, Novartis, Immunomedics, Changzhou Adam Biotech Inc, CN 102250246), TvAb (Roche, WO2012/025525, WO2012/025530), ScFv/Fc Fusions, SCORPION (Emergent BioSolutions/Trubion, Zymogenetics/BMS), Interceptor (Emergent), Dual Affinity Retargeting Technology (Fc-DART™) (MacroGenics, WO2008/157379, WO2010/080538), BEAT (Glenmark), Di-Diabody (Imclone/Eli Lilly) and chemically crosslinked mAbs (Karmanos Cancer Center), and covalently fused mAbs (AIMM therapeutics).

The term “monovalent antibody”, in the context of the present invention, refers to an antibody molecule that can interact with a specific epitope on an antigen, with only one antigen binding domain (e.g. one Fab arm). In the context of a bispecific antibody, “monovalent antibody binding” refers to the binding of the bispecific antibody to one specific epitope on an antigen with only one antigen binding domain (e.g. one Fab arm).

The term “monospecific antibody” in the context of the present invention, refers to an antibody that has binding specificity to one epitope only. The antibody may be a monospecific, monovalent antibody (i.e. carrying only one antigen binding region) or a monospecifc, bivalent antibody (i.e. an antibody with two identical antigen binding regions).

The term “bispecific antibody” refers to an antibody having two non-identical antigen binding domains, e.g. two non-identical Fab-arms or two Fab-arms with non-identical CDR regions. In the context of this invention, bispecific antibodies have specificity for at least two different epitopes. Such epitopes may be on the same or different antigens or targets. If the epitopes are on different antigens, such antigens may be on the same cell or different cells, cell types or structures, such as extracellular matrix or vesicles and soluble protein. A bispecific antibody may thus be capable of crosslinking multiple antigens, e.g. two different cells.

The term “bivalent antibody” refers to an antibody that has two antigen binding regions, which bind to epitopes on one or two targets or antigens or binds to one or two epitopes on the same antigen. Hence, a bivalent antibody may be a monospecific, bivalent antibody or a bispecific, bivalent antibody.

The terms “monoclonal antibody”, “monoclonal Ab”, “monoclonal antibody composition”, “mAb”, or the like, as used herein refer to a preparation of antibody molecules of single molecular composition. A monoclonal antibody composition displays a single binding specificity and affinity for a particular epitope. Accordingly, the term “human monoclonal antibody” refers to antibodies displaying a single binding specificity which have variable and constant regions derived from human germline immunoglobulin sequences. The human monoclonal antibodies may be produced by a hybridoma which includes a B cell obtained from a transgenic or transchromosomal non-human animal, such as a transgenic mouse, having a genome comprising a human heavy chain transgene and a light chain transgene, fused to an immortalized cell. Monoclonal antibodies may also be produced from recombinantly modified host cells, or systems that use cellular extracts supporting in vitro transcription and/or translation of nucleic acid sequences encoding the antibody.

The term “full-length antibody” when used herein, refers to an antibody (e.g., a parent or variant antibody) comprising one or two pairs of heavy and light chains, each containing all heavy and light chain constant and variable domains that are normally found in a heavy chain-light chain pair of a wild-type antibody of that isotype. In a full-length variant antibody, the heavy and light chain constant and variable domains may in particular contain amino acid substitutions that improve the functional properties of the antibody when compared to the full length parent or wild type antibody. A full-length antibody according to the present invention may be produced by a method comprising the steps of (i) cloning the CDR sequences into a suitable vector comprising complete heavy chain sequences and complete light chain sequence, and (ii) expressing the complete heavy and light chain sequences in suitable expression systems. It is within the knowledge of the skilled person to produce a full-length antibody when starting out from either CDR sequences or full variable region sequences. Thus, the skilled person would know how to generate a full-length antibody according to the present invention.

The term “chimeric antibody” as used herein, refers to an antibody wherein the variable region is derived from a non-human species (e.g. derived from rodents) and the constant region is derived from a different species, such as human. Chimeric monoclonal antibodies for therapeutic applications are developed to reduce antibody immunogenicity.

The term “human antibody”, as used herein, is intended to include antibodies having variable and framework regions derived from human germline immunoglobulin sequences and a human immunoglobulin constant domain. The human antibodies of the invention may include amino acid residues not encoded by human germline immunoglobulin sequences (e.g., mutations, insertions or deletions introduced by random or site-specific mutagenesis in vitro or by somatic mutation in vivo). However, the term “human antibody”, as used herein, is not intended to include antibodies in which CDR sequences derived from the germline of another non-human species, such as a mouse, have been grafted onto human framework sequences.

The term “humanized antibody” as used herein, refers to a genetically engineered non-human antibody, which contains human antibody constant domains and non-human variable domains modified to contain a high level of sequence homology to human variable domains. This can be achieved by grafting of the six non-human antibody complementarity-determining regions (CDRs), which together form the antigen binding site, onto a homologous human acceptor framework region (FR) (see WO92/22653 and EP0629240). In order to fully reconstitute the binding affinity and specificity of the parental antibody, the substitution of framework residues from the parental antibody (i.e. the non-human antibody) into the human framework regions (back-mutations) may be required. Structural homology modeling may help to identify the amino acid residues in the framework regions that are important for the binding properties of the antibody. Thus, a humanized antibody may comprise non-human CDR sequences, primarily human framework regions optionally comprising one or more amino acid back-mutations to the non-human amino acid sequence, and fully human constant regions. Optionally, additional amino acid modifications, which are not necessarily back-mutations, may be applied to obtain a humanized antibody with preferred characteristics, such as affinity and biochemical properties.

The term “Fc region” as used herein, refers to a region comprising, in the direction from the N- to C-terminal end of the antibody, at least a hinge region, a CH2 region and a CH3 region. An Fc region of the antibody may mediate the binding of the immunoglobulin to host tissues or factors, including various cells of the immune system (such as effector cells) and components of the complement system.

The term “hinge region” as used herein refers to the hinge region of an immunoglobulin heavy chain. Thus, for example the hinge region of a human IgG1 antibody corresponds to amino acids 216-230 according to the Eu numbering as set forth in Kabat Kabat, E. A. et al., Sequences of proteins of immunological interest. 5th Edition—US Department of Health and Human Services, NIH publication No. 91-3242, pp 662,680,689 (1991). However, the hinge region may also be any of the other subtypes as described herein.

The term “CH1 region” or “CH1 domain” as used herein refers to the CH1 region of an immunoglobulin heavy chain. Thus, for example the CH1 region of a human IgG1 antibody corresponds to amino acids 118-215 according to the Eu numbering as set forth in Kabat (ibid). However, the CH1 region may also be any of the other subtypes as described herein.

The term “CH2 region” or “CH2 domain” as used herein refers to the CH2 region of an immunoglobulin heavy chain. Thus, for example the CH2 region of a human IgG1 antibody corresponds to amino acids 231-340 according to the Eu numbering as set forth in Kabat (ibid). However, the CH2 region may also be any of the other subtypes as described herein.

The term “CH3 region” or “CH3 domain” as used herein refers to the CH3 region of an immunoglobulin heavy chain. Thus for example the CH3 region of a human IgG1 antibody corresponds to amino acids 341-447 according to the Eu numbering as set forth in Kabat (ibid). However, the CH3 region may also be any of the other subtypes as described herein.

The term “epitope” means a protein determinant capable of binding to an antigen-binding region of an antibody (“paratope”). Epitopes usually consist of surface groupings of molecules such as amino acids or sugar side chains and usually have specific three-dimensional structural characteristics, as well as specific charge characteristics. Conformational and non-conformational epitopes are distinguished in that the binding to the former, but not the latter, is lost in the presence of denaturing solvents. Epitope mapping techniques can determine “structural epitopes” or “functional epitopes”. Structural epitopes are defined as those residues within a structure that are in direct contact with the antibody and can for example be assessed by structure-based methods such as X-ray crystallography. A structural epitope may comprise amino acid residues directly involved in the binding of an antibody as well as other amino acid residues, which are not directly involved in the binding, such as amino acid residues which are effectively blocked or covered by antibody (in other words, the amino acid residue is within the footprint of the antibody). Functional epitope is defined as those residues that make energetic contributions to the antigen-antibody binding interaction and can for example be assessed by site-directed mutagenesis such as alanine scanning (Cunningham, B. C., & Wells, J. A. (1993) Journal of Molecular Biology; Clackson, T., & Wells, J. (1995) Science, 267(5196), 383-386). A functional epitope may comprise amino acid residues directly involved in the binding of an antibody as well as other amino acid residues which are not directly involved in the binding, such as amino acid residues which cause conformational changes to the location of residues involved in direct interactions (Greenspan, N. S., & Di Cera, E. (1999) Nature Biotechnology, 17(10), 936-937). In case of antibody-antigen interactions, the functional epitope may be used to distinguish antibody molecules between each other.

The term “Fc effector functions” or “Fc-mediated effector functions” as used herein, is intended to refer to functions that are a consequence of binding a polypeptide or antibody to its target, such as an antigen, on a cell membrane, and subsequent interaction of the IgG Fc domain with molecules of the innate immune system (e.g. soluble molecules or membrane-bound molecules). Examples of Fc effector functions include (i) C1q-binding, (ii) complement activation, (iii) complement-dependent cytotoxicity (CDC), (iv) antibody-dependent cell-mediated cytotoxicity (ADCC), (v) Fc-gamma receptor-binding, (vi) antibody-dependent cellular phagocytosis (ADCP), (vii) complement-dependent cellular cytotoxicity (CDCC), (viii) complement-enhanced cytotoxicity, (ix) binding to complement receptor of an opsonized antibody mediated by the antibody, (x) opsonisation, and (xi) a combination of any of (i) to (x).

The term “amino acid” and “amino acid residue” may herein be used interchangeably, and are not to be understood limiting. Amino acids are organic compounds containing amine (—NH₂) and carboxyl (—COOH) functional groups, along with a side chain (R group) specific to each amino acid. In the context of the present invention, amino acids may be classified based on structure and chemical characteristics. Thus, classes of amino acids may be reflected in one or both of the following tables:

Main classification based on structure and general chemical characterization of R group

| Class | Amino acid |
|---|---|
| Acidic Residues | D and E |
| Basic Residues | K, R, and H |
| Hydrophilic Uncharged Residues | S, T, N, and Q |
| Aliphatic Uncharged Residues | G, A, V, L, and I |
| Non-polar Uncharged Residues | C, M, and P |
| Aromatic Residues | F, Y, and W |

Alternative Physical and Functional Classifications of Amino Acid Residues

| Class | Amino acid |
|---|---|
| Hydroxyl group containing | S and T |
| residues | |
| Aliphatic residues | I, L, V, and M |
| Cycloalkenyl-associated | F, H, W, and Y |
| residues | |
| Hydrophobic residues | A, C, F, G, H, I, L, M, R, T, V, |
| | W, and Y |
| Negatively charged residues | D and E |
| Polar residues | C, D, E, H, K, N, Q, R, S, and T |
| Positively charged residues | H, K, and R |
| Small residues | A, C, D, G, N, P, S, T, and V |
| Very small residues | A, G, and S |
| Residues involved in turn | A, C, D, E, G, H, K, N, Q, R, S, |
| formation | P, and T |
| Flexible residues | Q, T, K, S, G, P, D, E, and R |

Substitution of one amino acid for another may be classified as a conservative or non-conservative substitution. In the context of the invention, a “conservative substitution” is a substitution of one amino acid with another amino acid having similar structural and/or chemical characteristics, such substitution of one amino acid residue for another amino acid residue of the same class as defined in any of the two tables above: for example, leucine may be substituted with isoleucine as they are both aliphatic, branched hydrophobes. Similarly, aspartic acid may be substituted with glutamic acid since they are both small, negatively charged residues.

In the context of the present invention, a substitution in an antibody is indicated as:
Original amino acid—position—substituted amino acid;

Referring to the well-recognized nomenclature for amino acids, the three letter code, or one letter code, is used, including the codes “Xaa” or“X” to indicate any amino acid residue. Thus, Xaa or X may typically represent any of the 20 naturally occurring amino acids. The term “naturally occurring” as used herein refers to any one of the following amino acid residues; glycine, alanine, valine, leucine, isoleucine, serine, threonine, lysine, arginine, histidine, aspartic acid, asparagine, glutamic acid, glutamine, proline, tryptophan, phenylalanine, tyrosine, methionine, and cysteine. Accordingly, the notation “K409R” or “Lys409Arg” means, that the antibody comprises a substitution of Lysine with Arginine in amino acid position 409.

Substitution of an amino acid at a given position to any other amino acid is referred to as:
Original amino acid—position; or e.g. “K409”

For a modification where the original amino acid(s) and/or substituted amino acid(s) may comprise more than one, but not all amino acid(s), the more than one amino acid may be separated by “,” or “/”. E.g. the substitution of Lysine with Arginine, Alanine, or Phenylalanine in position 409 is:

“Lys409Arg,Ala,Phe” or “Lys409Arg/Ala/Phe” or “K409R,A,F” or “K409R/A/F” or “K409 to R, A, or F”.

Such designation may be used interchangeably in the context of the invention but have the same meaning and purpose.

Furthermore, the term “a substitution” embraces a substitution into any one or the other nineteen natural amino acids, or into other amino acids, such as non-natural amino acids. For example, a substitution of amino acid K in position 409 includes each of the following substitutions: 409A, 409C, 409D, 409E, 409F, 409G, 409H, 409I, 409L, 409M, 409N, 409Q, 409R, 409S, 409T, 409V, 409W, 409P, and 409Y. This is, by the way, equivalent to the designation 409X, wherein the X designates any amino acid other than the original amino acid. These substitutions may also be designated K409A, K409C, etc. or K409A,C, etc. or K409A/C/etc. The same applies by analogy to each and every position mentioned herein, to specifically include herein any one of such substitutions.

The antibody according to the invention may also comprise a deletion of an amino acid residue. Such deletion may be denoted “del”, and includes, e.g., writing as K409del. Thus, in such embodiments, the Lysine in position 409 has been deleted from the amino acid sequence.

For purposes of the present invention, the “sequence identity” between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 5.0.0 or later. The parameters used are gap open penalty of 10, gap extension penalty of 0.5, and the EBLOSUM62 (EMBOSS version of BLOSUM62) substitution matrix. The output of Needle labeled “longest identity” (obtained using the -nobrief option) is used as the percent identity and is calculated as follows:

(Identical Residues×100)/(Length of Alignment−Total Number of Gaps in Alignment)

The retention of similar residues may also or alternatively be measured by a similarity score, as determined by use of a BLAST program (e.g., BLAST 2.2.8 available through the NCBI using standard settings BLOSUM62, Open Gap=11 and Extended Gap=1). Suitable variants typically exhibit at least about 45%, such as at least about 55%, at least about 65%, at least about 75%, at least about 85%, at least about 90%, at least about 95%, or more (e.g., about 99%) similarity to the parent sequence.

In the context of the present invention, “inhibition of PD-L1 binding to PD-1” refers to any detectably significant reduction in the binding of PD-L1 to PD-1 in the presence of an antibody capable of binding PD-L1. Typically, inhibition means an at least about 10% reduction, such as an at least about 15%, e.g. an at least about 20%, such as an at least 40% reduction in binding between PD-L1 and PD-1, caused by the presence of an anti-PD-L1 antibody. Inhibition of PD-L1 binding to PD-1 may be determined by any suitable technique. In one embodiment, inhibition is determined as described in Example 6 herein.

The term “treatment” refers to the administration of an effective amount of a pharmaceutical composition of the present invention with the purpose of easing, ameliorating, arresting or eradicating (curing) symptoms or disease states.

The term “effective amount” or “therapeutically effective amount” refers to an amount effective, at dosages and for periods of time necessary, to achieve a desired therapeutic result. A therapeutically effective amount of a binding agent, such as an antibody, in particular a bispecific antibody, may vary according to factors such as the disease state, age, sex, and weight of the individual, and the ability of the binding agent to elicit a desired response in the individual. A therapeutically effective amount is also one in which any toxic or detrimental effects of the antibody or antibody portion are outweighed by the therapeutically beneficial effects.

In a first aspect, the present invention relates to a pharmaceutical formulation comprising
a. a binding agent comprising a first antigen-binding region binding to human CD137 (4-1BB) and a second antigen-binding region binding to human PD-L1 (CD274),
   the first antigen biding region comprising a first heavy chain variable region (VH) comprising the three complementarity determining regions, CDR1, CDR2, and CDR3, present within the amino acid sequence set forth in SEQ ID NO: 15, and a first light chain variable region (VL) comprising the three complementarity determining regions, CDR1, CDR2, and CDR3, present within the amino acid sequence set forth in SEQ ID NO: 16, and
   the second antigen-binding region comprising a second heavy chain variable region (VH) comprising the three complementarity determining regions, CDR1, CDR2, and CDR3, present within the amino acid sequence set forth in SEQ ID NO: 17, and a second light chain variable region (VL) comprising the three complementarity determining regions, CDR1, CDR2, and CDR3, present within the amino acid sequence set forth in SEQ ID NO: 21;
b. a histidine buffer,
c. about 100 to about 400 mM of a sugar, and
d. about 0.001 to about 0.1% (w/v) non-ionic surfactant;
   and having a pH between about 4.5 and about 6.5.

The binding agent comprised in the pharmaceutical formulation according to the present invention may activate and/or induce proliferation in one cell by binding to CD137, while simultaneously binding to PD-L1 on another cell. In humans, CD137 is expressed on activated T cells, such as CD⁸⁺ T cells and CD⁴⁺ T cells, whereas PD-L1 is predominantly expressed on antigen-presenting cells (APCs) such as dendritic cells or tumor cells. Thus, binding agents, such as bispecific antibodies, according to the present invention capable of binding both CD137 and PD-L1 are able to simultaneously bind to T cells and APCs or T cells and tumor cells. Thus, binding agent in the formulation according to the invention may mediate cell-to-cell interaction between APCs and T cells by simultaneous binding of PD-L1 and CD137 on the cells. Thus, this may lead to proliferation of antigen-specific T cells. Further, the binding agent present in the formulation according to the invention may mediate cell-to-cell interaction between tumor cells and T cells by simultaneous binding of PD-L1 on tumor cells and CD137 on T cells. Thus, this may lead to further activation of T cells in the presence of tumor cells by binding of CD137 on the T cell, while binding of PD-L1 on tumor cells brings the T cell and tumor cell into close proximity. Thus, activation of T cells in the presence of tumor cells may lead to enhanced killing of tumor cells by the T cells. Further, the ability of the PD-L1 antigen-binding region, of the binding agent in the formulation according to the invention, to inhibit binding of PD-L1 on tumor cells with PD-1 on T cells prevents that the tumor cell is able to induce T cell inhibition, and thereby escaping the anti-tumor effect of the activated T cell.

Thus, a binding agent, such as a bispecific antibody, of the present invention may be used for treatment of a disease which can benefit from re-activation of T cells, such as cancer.

The pharmaceutical formulation may comprise 1 to 100 mM histidine, such as 5 to 100 mM, 10 to 100 mM, 15 to 100 mM, 5 to 90 mM, 5 to 80 mM, 5 to 70 mM, 5 to 60 mM, 5 to 50 mM, 5 to 40 mM, 5 to 30 mM, 10 to 90 mM, 10 to 80 mM, 10 to 70 mM, 10 to 60 mM, 10 to 50 mM, 10 to 40 mM, 10 to 30 mM, 15 to 90 mM, 15 to 80 mM, 15 to 70 mM, 15 to 60 mM, 15 to 50 mM, 15 to 40 mM, 15 to 30 mM or 15 to 20 mM histidine.

The pharmaceutical formulation may in particular comprise about 20 mM Histidine, such as 20 mM Histidine.

The pharmaceutical formulation may comprise 100 to 400 mM sugar, such as 125 to 400 mM, 150 to 400 mM, 150 to 400 mM, 175 to 400 mM, 200 to 400 mM, 225 to 400 mM, 100 to 375 mM, 100 to 350 mM, 100 to 325 mM, 100 to 300 mM, 125 to 375 mM, 125 to 350 mM, 125 to 325 mM, 125 to 300 mM, 125 to 275 mM, 150 to 375 mM, 150 to 350 mM, 150 to 325 mM, 150 to 300 mM, 150 to 275 mM, 175 to 375 mM, 175 to 350 mM, 175 to 325 mM, 175 to 300 mM, 175 to 275 mM, 200 to 375 mM 1, 200 to 350 mM 1, 200 to 325 mM, 200 to 300 mM, 200 to 275 mM, 225 to 375 mM, 225 to 350 mM, 225 to 325 mM, 225 to 300 mM, or such as 225 to 275 mM sugar.

In particular, the pharmaceutical formulation may comprise about 250 mM sugar, such as 250 mM sugar. Exemplary sugars include glucose, galactose, sucrose and trehalose dehydrate. The sugar may in particular be is sucrose.

The pharmaceutical formulation as disclosed herein may comprise 0.005 to 0.1% (w/v) non-ionic surfactant, such as 0.01 to 0.1% (w/v), 0.015 to 0.1% (w/v), 0.001 to 0.09% (w/v), 0.001 to 0.08% (w/v), 0.001 to 0.07% (w/v), 0.001 to 0.06% (w/v), 0.001 to 0.05% (w/v), 0.001 to 0.04% (w/v), 0.001 to 0.02% (w/v), 0.005 to 0.1% (w/v), 0.005 to 0.09% (w/v), 0.005 to 0.08% (w/v), 0.005 to 0.07% (w/v), 0.005 to 0.06% (w/v), 0.005 to 0.05% (w/v), 0.005 to 0.04% (w/v), 0.005 to 0.03% (w/v), 0.005 to 0.02% (w/v), 0.01 to 0.09% (w/v), 0.01 to 0.08% (w/v), 0.01 to 0.07% (w/v), 0.01 to 0.06% (w/v), 0.01 to 0.05% (w/v), 0.01 to 0.04% (w/v), 0.01 to 0.03% (w/v), 0.01 to 0.02% (w/v), 0.015 to 0.09% (w/v), 0.015 to 0.08% (w/v), 0.015 to 0.07% (w/v), 0.015 to 0.06% (w/v), 0.015 to 0.05% (w/v), 0.015 to 0.04% (w/v), 0.015 to 0.03% (w/v), or such as 0.015 to 0.02% (w/v) non-ionic surfactant.

In particular, the pharmaceutical formulation may comprise about 0.02% (w/v) non-ionic surfactant, such as 0.02% (w/v) non-ionic surfactant.

The non-ionic surfactant may be selected from 2-[2-[3,4-bis(2-hydroxyethoxy)oxolan-2-yl]-2-(2-hydroxyethoxy)ethoxy]ethyl (E)-octadec-9-enoate (Polyoxyethylene (20) sorbitan monooleate; Polysorbate 80) or 2-[2-[3,4-bis(2-hydroxyethoxy)oxolan-2-yl]-2-(2-hydroxyethoxy)ethoxy]ethyl dodecanoate (Polyoxyethylene (20) sorbitan monolaurate; Polysorbate 20).

The pharmaceutical formulation may have a pH between 4.5 and 6.5, such as between 4.7 and 6.5, e.g. between 4.9 and 6.5, between 5.1 and 6.5, between 5.3 and 6.5, between 4.5 and 6.3, between 4.7 and 6.1, between 4.7 and 5.9, between 4.7 and 5.7, between 5.1 and 6.3, between 4.7 and 6.1, between 4.7 and 5.9, between 4.7 and 5.7, between 4.9 and 6.3, between 4.9 and 6.1, between 4.9 and 5.9, between 4.9 and 5.7, between 5.1 and 6.3, between 5.1 and 6.1, between 5.1 and 5.9, between 5.1 and 5.7, between 5.3 and 6.3, between 5.3 and 6.1, between 5.3 and 5.9, such as between 5.3 and 5.7.

In currently preferred embodiments, the pharmaceutical formulation according to the invention has a pH, which is about 5.5, such as a pH of 5.5.

The pharmaceutical formulation may comprise 5 to 200 mg/mL of the binding agent, such as 10 to 200 mg/mL, 20 to 200 mg/mL, 40 to 200 mg/mL, 60 to 200 mg/mL, 80 to 200 mg/mL, 100 to 200 mg/mL, 120 to 200 mg/mL, 150 to 200 mg/mL, 5 to 150 mg/mL, 10 to 150 mg/mL, 20 to 150 mg/mL, 40 to 150 mg/mL, 60 to 150 mg/mL, 80 to 150 mg/mL, 100 to 150 mg/mL, 5 to 130 mg/mL, 10 to 130 mg/mL, 20 to 130 mg/mL, 40 to 130 mg/mL, 60 to 130 mg/mL, 80 to 130 mg/mL, 100 to 130 mg/mL, 5 to 100 mg/mL of the binding agent, 10 to 100 mg/mL, 15 to 100 mg/mL, 20 to 100 mg/mL, 30 to 100 mg/mL, 40 to 100 mg/mL, 50 to 100 mg/mL, 60 to 100 mg/mL, 5 to 80 mg/mL, 5 to 60 mg/mL, 5 to 50 mg/mL, 5 to 40 mg/mL, 5 to 30 mg/mL, 5 to 20 mg/mL, 10 to 80 mg/mL, 10 to 60 mg/mL, 10 to 50 mg/mL, 10 to 40 mg/mL, 10 to 30 mg/mL, 15 to 80 mg/mL, 15 to 60 mg/mL, 15 to 40 mg/mL, or such as 15 to 25 mg/mL of the binding agent.

The pharmaceutical formulation according to any one of the preceding claims, wherein the formulation comprises
i) about 20 mg/mL of the binding agent, such as about 40 mg/mL, about 60 mg/mL, about 80 mg/mL, about 100 mg/mL, about 120 mg/mL, or about 140 mg/mL, and
ii) about 20 mM Histidine, about 250 mM sugar, and about 0.02% (w/v) non-ionic surfactant and has a pH about 5.5.

In particular, the pharmaceutical formulation provided herein may comprise about 20 mg/mL of the binding agent, such as 20 mg/mL of the binding agent.

The formulation may in particular comprise about 20 mg/mL of the binding agent, about 20 mM Histidine, about 250 mM sugar, and about 0.02% (w/v) non-ionic surfactant and has a pH about 5.5.

The pharmaceutical formulation may comprise:
i) 20 mg/mL of the binding agent such as 40 mg/mL, 60 mg/mL, 80 mg/mL, 100 mg/mL, 120 mg/mL, or 140 mg/mL, and
ii) 20 mM Histidine, 250 mM sugar, and 0.02% (w/v) non-ionic surfactant and has a pH of 5.5.

In one embodiment, which is currently preferred, the pharmaceutical formulation according to the invention comprises 20 mg/mL of the binding agent, 20 mM Histidine, 250 mM sugar, and 0.02% (w/v) non-ionic surfactant and has a pH of 5.5.

Preferably, the pharmaceutical formulation according to the invention is essentially free of visible particles after having been subjected to 5 freeze-thaw cycles consisting of freezing for 12 h at −65° C. following by thawing for 12 h at 25° C., as determined by visible particle count performed against a black background and against a white background at an illumination of an intensity between 2000 and 3750 lux.

The binding agent comprised by the pharmaceutical formulation may in particular be an antibody, such as a bispecific antibody.

Each of the variable regions defined above may comprise three complementarity determining regions, CDR1, CDR2, and CDR3, and four framework regions, FR1, FR2, FR3, and FR4.

In the pharmaceutical formulation, the said complementarity determining regions and said framework regions are arranged from amino-terminus to carboxy-terminus in the following order: FR1, CDR1, FR2, CDR2, FR3, CDR3, FR4.

In one embodiment of the invention, the binding agent, in particular in the form of an antibody, such as a bispecific antibody, comprises a heavy chain variable region, wherein the complementary determining regions and the framework regions are arranged from the amino-terminus to the carboxy-terminus in the following order: HFR1, HCDR1, HFR2, HCDR2, HFR3, HCDR3, HFR4.

In one embodiment of the invention, the binding agent, in particular in the form of an antibody such as a bispecific antibody, comprises a light chain variable region, wherein the complementary determining regions and the framework regions are arranged from the amino-terminus to the carboxy-terminus in the following order: LFR1, LCDR1, LFR2, LCDR2, LFR3, LCDR3, LFR4.

In the pharmaceutical formulation
the first antigen biding region may comprise a first heavy chain variable region (VH) comprising the CDR1, CDR2, and CDR3 sequences set forth in: SEQ ID NO: 9, 10, 11, respectively, and a first light chain variable region (VL) comprising the CDR1, CDR2, and CDR3 sequences as set forth in: SEQ ID NO: 13, GAS, and SEQ ID NO: 14, respectively, and
the second antigen-binding region may comprise a second heavy chain variable region (VH) comprising the CDR1, CDR2, and CDR3 sequences set forth in: SEQ ID NO: 18, 19 and 20 respectively, and a second light chain variable region (VL) comprising the CDR1, CDR2, and CDR3 sequences set forth in: SEQ ID NO: 22, DDN and SEQ ID NO: 23, respectively.

The present invention also provides formulations in which the antibody comprises heavy and light chain variable regions as disclosed in the examples of the present application. Also provided are formulations of antibodies comprising functional variants of the VL regions, VH regions disclosed in the examples. A functional variant of a VL or VH used in the context of an antibody still allows the antibody to retain at least a substantial proportion (at least about 50%, 60%, 70%, 80%, 90%, 95% or more) of the affinity and/or the specificity/selectivity of the “reference” or “parent” antibody and in some cases, such an antibody may be associated with greater affinity, selectivity and/or specificity than the parent antibody. Such functional variants typically retain significant sequence identity to the parent antibody.

Hence, the pharmaceutical formulation according to the invention may be one wherein
the first antigen biding region comprises a first heavy chain variable region (VH) having at least 70%, at least 75%, at least 80%, at least 85%, at least 90%, at least 95%, at least 97%, at least 99%, or 100% sequence identity to the sequence set forth in SEQ ID NO: 15; and a first light chain variable region (VL) having at least 70%, at least 75%, at least 80%, at least 85%, at least 90%, at least 95%, at least 97%, at least 99%, or 100% sequence identity to the sequence set forth in SEQ ID NO: 16; and
the second antigen-binding region comprises a second heavy chain variable region (VH) having at least 70%, at least 75%, at least 80%, at least 85%, at least 90%, at least 95%, at least 97%, at least 99%, or 100% sequence identity to the sequence set forth in SEQ ID NO: 17; and a second light chain variable region (VL) having at least 70%, at least 75%, at least 80%, at least 85%, at least 90%, at least 95%, at least 97%, at least 99%, or 100% sequence identity to the sequence set forth in SEQ ID NO: 21.

Further, the pharmaceutical formulation according to the present disclosure may be one wherein
the first antigen biding region comprises a first heavy chain variable region (VH) comprising a CDR1, CDR2, and CDR3 sequence, as set forth in: SEQ ID NO: 9, 10 and 11, respectively, the first heavy chain variable region having at least 70%, at least 75%, at least 80%, at least 85%, at least 90%, at least 95%, at least 97%, at least 99%, or 100% sequence identity to the sequence set forth in SEQ ID NO: 15; and a first light chain variable region (VL) comprising a CDR1, CDR2, and CDR3 sequence, as set forth in: SEQ ID NO: 13, GAS, and SEQ ID NO: 14, respectively, the first light chain variable region having at least 70%, at least 75%, at least 80%, at least 85%, at least 90%, at least 95%, at least 97%, at least 99%, or 100% sequence identity to the sequence set forth in SEQ ID NO: 16, and
the second antigen-binding region comprises a second heavy chain variable region (VH) comprising a CDR1, CDR2, and CDR3 sequence, as set forth in: SEQ ID NO: 18, 19 and 20, respectively, the second heavy chain variable region having at least 70%, at least 75%, at least 80%, at least 85%, at least 90%, at least 95%, at least 97%, at least 99%, or 100% sequence identity to the sequence set forth in SEQ ID NO: 17; and a second light chain variable region (VL) comprising a CDR1, CDR2, and CDR3 sequence, as set forth in: SEQ ID NO: 22, DDN, 23, respectively, the second light chain variable region having at least 70%, at least 75%, at least 80%, at least 85%, at least 90%, at least 95%, at least 97%, at least 99%, or 100% sequence identity to the sequence set forth in SEQ ID NO: 21.

The pharmaceutical formulation according to the invention may be one wherein:
a. said first antigen-binding region binding to human CD137 comprises
   a first heavy chain variable region comprising the sequence set forth in SEQ ID NO: 15 or a sequence wherein up to 20 amino acid residues, such as up to 19, up to 18, up to 17, up to 16, up to 15, up to 14, up to 13, up to 12, up to 11, up to 10, up to 9, up to 8, up to 7, up to 6, up to 5, up to 4, up to 3, up to 2, up to 1 amino acid residues is/are modified as compared to the sequence set forth in SEQ ID NO: 15, the first heavy chain variable region (VH) comprising a CDR1, CDR2, and CDR3 sequence, as set forth in: SEQ ID NO: 9, 10 and 11, respectively; and
   a first light chain variable region comprising the sequence set forth in SEQ ID NO: 16 or a sequence wherein up to 20 amino acid residues, such as up to 19, up to 18, up to 17, up to 16, up to 15, up to 14, up to 13, up to 12, up to 11, up to 10, up to 9, up to 8, up to 7, up to 6, up to 5, up to 4, up to 3, up to 2, up to 1 amino acid residues is/are modified as compared to the sequence set forth in SEQ ID NO: 16 the first light chain variable region (VL) comprising a CDR1, CDR2, and CDR3 sequence, as set forth in: SEQ ID NO: 13, GAS and SEQ ID NO: 14, respectively; and
b. said second antigen-binding region binding to human PD-L1 comprises
   a second heavy chain variable region comprising the sequence set forth in SEQ ID NO: 17 or a sequence wherein up to 20 amino acid residues, such as up to 19, up to 18, up to 17, up to 16, up to 15, up to 14, up to 13, up to 12, up to 11, up to 10, up to 9, up to 8, up to 7, up to 6, up to 5, up to 4, up to 3, up to 2, up to 1 amino acid residues is/are modified as compared to the sequence set forth in SEQ ID NO: 17, the second heavy chain variable region (VH) comprising a CDR1, CDR2, and CDR3 sequence, as set forth in: SEQ ID NO: 18, 19 and 20, respectively; and
   a second light chain variable region comprising the sequence set forth in SEQ ID NO: 21 or a sequence wherein up to 20 amino acid residues, such as up to 19, up to 18, up to 17, up to 16, up to 15, up to 14, up to 13, up to 12, up to 11, up to 10, up to 9, up to 8, up to 7, up to 6, up to 5, up to 4, up to 3, up to 2, up to 1 amino acid residues is/are modified as compared to the sequence set forth in SEQ ID NO: 21, the second light chain variable region (VH) comprising a CDR1, CDR2, and CDR3 sequence, as set forth in: SEQ ID NO: 22, DDN and SEQ ID NO: 23, respectively.

In particular embodiments, the modification(s) of amino acid residues referred to above may be an amino acid substitution, such as a conservative amino acid substitution. Other modifications of amino acid residues comprised by the present disclosure include deletion of one or more amino acids as well as addition and/or insertion of one or more amino acid residues.

The present disclosure further provides a pharmaceutical formulation, wherein said binding agent comprises (i) a polypeptide comprising said first heavy chain variable region (VH) and further comprising a first heavy chain constant region (CH) and (ii) a polypeptide comprising said second heavy chain variable region (VH) and further comprising a second heavy chain constant region (CH).

The pharmaceutical composition as disclosed herein may comprise (i) a polypeptide comprising said first light chain variable region (VL) and further comprising a first light chain constant region (CL) and (ii) a polypeptide comprising said second light chain variable region (VL) and further comprising a second light chain constant region (CL).

The pharmaceutical formulation may comprise a biding agent, such as an antibody, comprising a first binding arm and a second binding arm, wherein
a. the first binding arm comprises i) a polypeptide comprising said first heavy chain variable region (VH) and said first heavy chain constant region (CH) and ii) a polypeptide comprising said first light chain variable region (VL) and said first light chain constant region (CL) and;
b. the second binding arm comprises i) a polypeptide comprising said second heavy chain variable region (VH) and said second heavy chain constant region (CH) and ii) a polypeptide comprising said second light chain variable region (VL) and said second light chain constant region (CL).

In particular embodiments of the invention, the first antigen-binding region binds to human CD137 as set forth in SEQ ID NO: 30, or a mature polypeptide thereof.

The first antigen-binding region may also be able to bind to cynomolgus monkey (Macaca fascicularis) CD137, as set forth in SEQ ID NO: 31, or a mature polypeptide thereof. An antigen binding region which is cross-specific for both human and cynomolgus monkey CD137 makes the binding agent in the pharmaceutical formulation suitable for preclinical testing in the cynomolgus monkey.

In the pharmaceutical formulation according to present disclosure, the second antigen-binding region preferably binds to human PD-L1 as set forth in SEQ ID NO: 28, or a mature polypeptide thereof.

The second antigen-binding region may also be able to bind to cynomolgus monkey (Macaca fascicularis) PD-L1 as set forth in SEQ ID NO: 29, or a mature polypeptide thereof.

Further, the second antigen-binding region may be able to inhibit the binding of human PD-L1 to human PD-1. This is of interest because the binding agent may thereby prevent PD-L1 from obstructing anti-tumor immunity through PD-1. Thus, the binding agent may prevent that the T cells receives an inhibitory signal through PD-1/PD-L1 interaction, while receiving an activation signal through binding to the CD137 molecule resulting in signaling that strengthens T cell proliferation, activation, effector and memory functions.

The binding agent may be in the format of a full-length antibody or an antibody fragment.

In particular, the binding agent, such as the antibody, may be of an isotype selected from the group consisting of IgG1, IgG2, IgG3, and IgG4.

According to the present disclosure the binding agent is a full-length IgG1 antibody.

In various embodiments, the antibody is an IgG1 antibody, more particularly an IgG1, kappa or IgG1, lambda isotype (i.e. IgG1, K, A), an IgG2a antibody (e.g. IgG2a, K, A), an IgG2b antibody (e.g. IgG2b, K, A), an IgG3 antibody (e.g. IgG3, K, A) or an IgG4 antibody (e.g. IgG4, K, A).

In the pharmaceutical formulation according to the present disclosure
a. the first antigen-binding region binding to CD137 may be derived from a chimeric antibody, and/or
b. the second antigen-binding region binding to human PD-L1 may be derived from a chimeric antibody.

Alternatively, in the pharmaceutical formulation according to the preceding disclosure,
a. the first antigen-binding region binding to CD137 may be derived from a humanized antibody, and/or
b. the second antigen-binding region binding to human PD-L1 may be derived from a humanized antibody.

As another alternative,
a. the first antigen-binding region binding to human CD137 may derived from a human antibody, and/or
b. the second antigen-binding region binding to human PD-L1 may derived from a human antibody.

In an even further alternative
a. the first antigen-binding region binding to human CD137 may be derived from a humanized antibody, and/or
b. the second antigen-binding region binding to human PD-L1 may be derived from a human antibody.

The first antigen-binding region may in particular be derived from a rabbit antibody. The first antigen-binding region may further be derived from a humanized antibody. Also, the first binding arm may be derived from a full-length antibody. In one embodiment of the invention, the first binding arm is derived from a monoclonal antibody. The first binding arm may be derived from a full-length IgG1, A (lambda) or IgG1, K (kappa) antibody.

The second antigen-binding region may be derived from a rat antibody. In one embodiment of the invention, the second antigen-binding region is human. Alternatively, the second antigen-binding region may be derived from a humanized antibody. Also, the second binding arm may be derived from a full-length antibody. In one embodiment of the invention, the second binding arm is derived from a monoclonal antibody. In one embodiment of the invention, the second binding arm is derived from a full-length IgG1, A (lambda) or IgG1, K (kappa) antibody. The first and second antigen-binding regions may be derived from humanized antibodies. The first and second antigen-binding regions may be human antibodies. The first and second binding arms may be derived from full-length antibodies, such as from full-length IgG1, A (lambda) or IgG1, K (kappa) antibodies. The first and second binding arms may be derived from monoclonal antibodies.

In one embodiment of the invention, the first antigen binding region is derived from an IgG1 lambda and the second antigen binding region is derived from an IgG1 kappa.

Many different formats and uses of bispecific antibodies are known in the art, and were reviewed by Kontermann; Drug Discov Today, 2015 July; 20(7):838-47 and; MAbs, 2012 March-April; 4(2): 182-97.

In embodiments of the present invention, in which the binding agent is a bispecific antibody the disclosure is not limited to any particular bispecific format or method of producing it.

Examples of bispecific antibody molecules which may be used in the present invention comprise (i) a single antibody that has two arms comprising different antigen-binding regions; (ii) a single chain antibody that has specificity to two different epitopes, e.g., via two scFvs linked in tandem by an extra peptide linker; (iii) a dual-variable-domain antibody (DVD-Ig), where each light chain and heavy chain contains two variable domains in tandem through a short peptide linkage (Wu et al., Generation and Characterization of a Dual Variable Domain Immunoglobulin (DVD-Ig™) Molecule, In: Antibody Engineering, Springer Berlin Heidelberg (2010)); (iv) a chemically-linked bispecific (Fab′)2 fragment; (v) a Tandab, which is a fusion of two single chain diabodies resulting in a tetravalent bispecific antibody that has two binding sites for each of the target antigens; (vi) a flexibody, which is a combination of scFvs with a diabody resulting in a multivalent molecule; (vii) a so-called “dock and lock” molecule, based on the “dimerization and docking domain” in Protein Kinase A, which, when applied to Fabs, can yield a trivalent bispecific binding protein consisting of two identical Fab fragments linked to a different Fab fragment; (viii) a so-called Scorpion molecule, comprising, e.g., two scFvs fused to both termini of a human Fab-arm; and (ix) a diabody.

The binding agent of the present invention may for example be a diabody or a cross-body.

In one embodiment, the binding agent of the invention is a bispecific antibody obtained via a controlled Fab-arm exchange (such as described in WO2011131746 (Genmab)).

Examples of different classes of binding agents which may be applicable in the content of the present invention include but are not limited to (i) IgG-like molecules with complementary CH3 domains to force heterodimerization; (ii) recombinant molecules include but are not limited to the Triomab/Quadroma molecules (Trion Pharma/Fresenius Biotech; Roche, WO2011069104), the so-called Knobs-into-Holes molecules (Genentech, WO9850431), CrossMAbs (Roche, WO2011117329) and the electrostatically-matched molecules (Amgen, EP1870459 and WO2009089004; Chugai, US201000155133; Oncomed, WO2010129304), the LUZ-Y molecules (Genentech, Wranik et al. J. Biol. Chem. 2012, 287(52): 43331-9, doi: 10.1074/jbc.M112.397869. Epub 2012 Nov. 1), DIG-body and PIG-body molecules (Pharmabcine, WO2010134666, WO2014081202), the Strand Exchange Engineered Domain body (SEEDbody) molecules (EMD Serono, WO2007110205), the Biclonics molecules (Merus, WO2013157953), FcAAdp molecules (Regeneron, WO201015792), bispecific IgG1 and IgG2 molecules (Pfizer/Rinat, WO11143545), Azymetric scaffold molecules (Zymeworks/Merck, WO2012058768), mAb-Fv molecules (Xencor, WO2011028952), bivalent bispecific antibodies (WO2009080254) and the DuoBody® molecules (Genmab, WO2011131746).

Examples of recombinant IgG-like dual targeting molecules include but are not limited to Dual Targeting (DT)-Ig molecules (WO2009058383), Two-in-one Antibody (Genentech; Bostrom, et al 2009. Science 323, 1610-1614), Cross-linked Mabs (Karmanos Cancer Center), mAb2 (F-Star, WO2008003116), Zybody molecules (Zyngenia; LaFleur et al. MAbs. 2013 March-April; 5(2):208-18), approaches with common light chain (Crucell/Merus, U.S. Pat. No. 7,262,028), KABodies (NovImmune, WO2012023053) and CovX-body (CovX/Pfizer; Doppalapudi, V. R., et al 2007. Bioorg. Med. Chem. Lett. 17, 501-506).

Examples of IgG fusion molecules include but are not limited to Dual Variable Domain (DVD)-Ig molecules (Abbott, U.S. Pat. No. 7,612,181), Dual domain double head antibodies (Unilever; Sanofi Aventis, WO20100226923), IgG-like Bispecific molecules (ImClone/Eli Lilly, Lewis et al. Nat Biotechnol. 2014 February; 32(2):191-8), Ts2Ab (MedImmune/AZ; Dimasi et al. J Mol Biol. 2009 Oct. 30; 393(3):672-92) and BsAb molecules (Zymogenetics, WO2010111625), HERCULES molecules (Biogen Idec, U.S. Ser. No. 00/795,1918), scFv fusion molecules (Novartis), scFv fusion molecules (Changzhou Adam Biotech Inc, CN 102250246) and TvAb molecules (Roche, WO2012025525, WO2012025530).

Examples of Fc fusion molecules include but are not limited to ScFv/Fc Fusions (Pearce et al., Biochem Mol Biol Int. 1997 September; 42(6):1179-88), SCORPION molecules (Emergent BioSolutions/Trubion, Blankenship J W, et al. AACR 100th Annual meeting 2009 (Abstract #5465); Zymogenetics/BMS, WO2010111625), Dual Affinity Retargeting Technology (Fc-DART) molecules (MacroGenics, WO2008157379, WO2010080538) and Dual(ScFv)2-Fab molecules (National Research Center for Antibody Medicine—China).

Examples of Fab fusion bispecific antibodies include but are not limited to F(ab)2 molecules (Medarex/AMGEN; Deo et al J Immunol. 1998 Feb. 15; 160(4):1677-86), Dual-Action or Bis-Fab molecules (Genentech, Bostrom, et al 2009. Science 323, 1610-1614), Dock-and-Lock (DNL) molecules (ImmunoMedics, WO2003074569, WO2005004809), Bivalent Bispecific molecules (Biotecnol, Schoonjans, J Immunol. 2000 Dec. 15; 165(12):7050-7) and Fab-Fv molecules (UCB-Celltech, WO 2009040562 A1).

Examples of ScFv-, diabody-based and domain antibodies include but are not limited to Bispecific T Cell Engager (BITE) molecules (Micromet, WO2005061547), Tandem Diabody molecules (TandAb) (Affimed) Le Gall et al., Protein Eng Des Sel. 2004 April; 17(4):357-66), Dual Affinity Retargeting Technology (DART) molecules (MacroGenics, WO2008157379, WO2010080538), Single-chain Diabody molecules (Lawrence, FEBS Lett. 1998 Apr. 3; 425(3):479-84), TCR-like Antibodies (AIT, ReceptorLogics), Human Serum Albumin ScFv Fusion (Merrimack, WO2010059315) and COMBODY molecules (Epigen Biotech, Zhu et al. Immunol Cell Biol. 2010 August; 88(6):667-75), dual targeting nanobodies (Ablynx, Hmila et al., FASEB J. 2010) and dual targeting heavy chain only domain antibodies.

Each of the first and second heavy chain constant regions (CH) may comprise one or more of a constant region domain 1 region (CH1 region), a hinge region, a CH2 region and a CH3 region, preferably at least a hinge region, a CH2 region and a CH3 region.

The binding agent, such as the bispecific antibody, of the present disclosure may comprise a first Fc sequence comprising a first CH3 region, and a second Fc sequence comprising a second CH3 region, wherein the sequences of the first and second CH3 regions are different and are such that the heterodimeric interaction between said first and second CH3 regions is stronger than each of the homodimeric interactions of said first and second CH3 regions. More details on these interactions and how they can be achieved are provided in WO2011131746 and WO2013060867 (Genmab), which are hereby incorporated by reference.

As described further herein, a stable bispecific PD-L1xCD137 antibody can be obtained at high yield using a particular method on the basis of one homodimeric starting PD-L1 antibody and one homodimeric starting CD137 antibody containing only a few, conservative, asymmetrical mutations in the CH3 regions. Asymmetrical mutations mean that the sequences of said first and second CH3 regions contain amino acid substitutions at non-identical positions.

Hence, in one embodiment of the invention, each of the first and second heavy chain constant regions (CHs) comprises a CH3 region, the two CH3 regions comprising asymmetrical mutations.

In the pharmaceutical formulation according to the disclosure, may comprise a binding agent, wherein in said first heavy chain constant region (CH) at least one of the amino acids in a position corresponding to a position selected from the group consisting of T366, L368, K370, D399, F405, Y407, and K409 in a human IgG1 heavy chain according to EU numbering has been substituted, and in said second heavy chain constant region (CH) at least one of the amino acids in a position corresponding to a position selected from the group consisting of T366, L368, K370, D399, F405, Y407, and K409 in a human IgG1 heavy chain according to EU numbering has been substituted, and wherein said first and said second heavy chains are not substituted in the same positions.

The pharmaceutical formulation disclosed herein may comprise a binding agent, wherein (i) the amino acid in the position corresponding to F405 in a human IgG1 heavy chain according to EU numbering is L in said first heavy chain constant region (CH), and the amino acid in the position corresponding to K409 in a human IgG1 heavy chain according to EU numbering is R in said second heavy chain constant region (CH), or (ii) the amino acid in the position corresponding to K409 in a human IgG1 heavy chain according to EU numbering is R in said first heavy chain, and the amino acid in the position corresponding to F405 in a human IgG1 heavy chain according to EU numbering is L in said second heavy chain.

The bispecific antibody disclosed herein, may comprise a first CH3 region which has an amino acid substitution at position 366 in a human IgG1 heavy chain, and a second CH3 region which has an amino acid substitution at a position selected from the group consisting of: 368, 370, 399, 405, 407 and 409 in a human IgG1 heavy chain. The amino acid at position 366 in a human IgG1 heavy chain may be selected from Ala, Asp, Glu, His, Asn, Val, or Gln.

The bispecific antibody disclosed herein may comprise a first CH3 region, which has an amino acid substitution at position 368 in a human IgG1 heavy chain, and a second CH3 region which has an amino acid substitution at a position selected from the group consisting of: 366, 370, 399, 405, 407 and 409 in a human IgG1 heavy chain.

The bispecific antibody disclosed herein may comprise a first CH3 region, which has an amino acid substitution at position 370 in a human IgG1 heavy chain, and a second CH3 region which has an amino acid substitution at a position selected from the group consisting of: 366, 368, 399, 405, 407 and 409 in a human IgG1 heavy chain.

The bispecific antibody disclosed herein may comprise a first CH3 region which has an amino acid substitution at position 399 in a human IgG1 heavy chain, and a second CH3 region which has an amino acid substitution at a position selected from the group consisting of: 366, 368, 370, 405, 407 and 409 in a human IgG1 heavy chain.

The bispecific antibody disclosed herein may comprise a first CH3 region, which has an amino acid substitution at position 405 in a human IgG1 heavy chain, and a second CH3 region which has an amino acid substitution at a position selected from the group consisting of: 366, 368, 370, 399, 407 and 409 in a human IgG1 heavy chain.

The bispecific antibody disclosed herein may comprise a first CH3 region which has an amino acid substitution at position 407 in a human IgG1 heavy chain, and a second CH3 region which has an amino acid substitution at a position selected from the group consisting of: 366, 368, 370, 399, 405, and 409 in a human IgG1 heavy chain.

The bispecific antibody disclosed herein may comprise a first CH3 region which has an amino acid substitution at position 409 in a human IgG1 heavy chain, and a second CH3 region has an amino acid substitution at a position selected from the group consisting of: 366, 368, 370, 399, 405, and 407 in a human IgG1 heavy chain.

Accordingly, the bispecific antibody disclosed herein may comprise the sequences of said first and second CH3 regions contain asymmetrical mutations, i.e. mutations at different positions in the two CH3 regions, e.g. a mutation at position 405 in one of the CH3 regions and a mutation at position 409 in the other CH3 region.

The bispecific antibody disclosed herein may be an antibody wherein the first CH3 region has an amino acid other than Lys, Leu or Met, e.g. Gly, Ala, Val, Ile, Ser, Thr, Phe, Arg, His, Asp, Asn, Glu, Gln, Pro, Trp, Tyr, or Cys, at position 409 and said second CH3 region has an amino-acid substitution at a position selected from the group consisting of: 366, 368, 370, 399, 405 and 407. The first CH3 region may have an amino acid other than Lys, Leu or Met, e.g. Gly, Ala, Val, Ile, Ser, Thr, Phe, Arg, His, Asp, Asn, Glu, Gln, Pro, Trp, Tyr, or Cys, at position 409 and said second CH3 region may have an amino acid other than Phe, e.g. Gly, Ala, Val, Ile, Ser, Thr, Lys, Arg, His, Asp, Asn, Glu, Gln, Pro, Trp, Tyr, Cys, Lys, or Leu, at position 405. In a further embodiment hereof, said first CH3 region may have an amino acid other than Lys, Leu or Met, e.g. Gly, Ala, Val, Ile, Ser, Thr, Phe, Arg, His, Asp, Asn, Glu, Gln, Pro, Trp, Tyr, or Cys, at position 409 and said second CH3 region may have an amino acid other than Phe, Arg or Gly, e.g. Leu, Ala, Val, Ile, Ser, Thr, Met, Lys, His, Asp, Asn, Glu, Gln, Pro, Trp, Tyr, or Cys, at position 405.

The bispecific antibody disclosed herein may be an antibody, wherein said first CH3 region comprises a Phe at position 405 and an amino acid other than Lys, Leu or Met, e.g. Gly, Ala, Val, Ile, Ser, Thr, Phe, Arg, His, Asp, Asn, Glu, Gln, Pro, Trp, Tyr, or Cys, at position 409 and said second CH3 region comprises an amino acid other than Phe, e.g. Gly, Ala, Val, Ile, Ser, Thr, Lys, Arg, His, Asp, Asn, Glu, Gln, Pro, Trp, Tyr, Leu, Met, or Cys, at position 405 and a Lys at position 409. The first CH3 region may comprise a Phe at position 405 and an amino acid other than Lys, Leu or Met, e.g. Gly, Ala, Val, Ile, Ser, Thr, Phe, Arg, His, Asp, Asn, Glu, Gln, Pro, Trp, Tyr, or Cys, at position 409 and the second CH3 region may comprise an amino acid other than Phe, Arg or Gly, e.g. Leu, Ala, Val, Ile, Ser, Thr, Met, Lys, His, Asp, Asn, Glu, Gln, Pro, Trp, Tyr, or Cys, at position 405 and a Lys at position 409.

The bispecific antibody disclosed herein may be an antibody, wherein said first CH3 region comprises a Phe at position 405 and an amino acid other than Lys, Leu or Met, e.g. Gly, Ala, Val, Ile, Ser, Thr, Phe, Arg, His, Asp, Asn, Glu, Gln, Pro, Trp, Tyr, or Cys, at position 409 and said second CH3 region comprises a Leu at position 405 and a Lys at position 409. The first CH3 region may comprise a Phe at position 405 and an Arg at position 409 and said second CH3 region comprises an amino acid other than Phe, Arg or Gly, e.g. Leu, Ala, Val, Ile, Ser, Thr, Lys, Met, His, Asp, Asn, Glu, Gln, Pro, Trp, Tyr, or Cys, at position 405 and a Lys at position 409. The first CH3 region may comprise Phe at position 405 and an Arg at position 409 and said second CH3 region comprises a Leu at position 405 and a Lys at position 409.

The bispecific antibody disclosed herein may be an antibody, wherein said first CH3 region comprises an amino acid other than Lys, Leu or Met, e.g. Gly, Ala, Val, Ile, Ser, Thr, Phe, Arg, His, Asp, Asn, Glu, Gln, Pro, Trp, Tyr, or Cys, at position 409 and said second CH3 region comprises a Lys at position 409, a Thr at position 370 and a Leu at position 405. The first CH3 region may comprise an Arg at position 409 and said second CH3 region may comprise a Lys at position 409, a Thr at position 370 and a Leu at position 405.

The bispecific antibody disclosed herein may be an antibody, wherein said first CH3 region comprises a Lys at position 370, a Phe at position 405 and an Arg at position 409 and said second CH3 region comprises a Lys at position 409, a Thr at position 370 and a Leu at position 405.

The bispecific antibody disclosed herein may be an antibody, wherein said first CH3 region comprises an amino acid other than Lys, Leu or Met, e.g. Gly, Ala, Val, Ile, Ser, Thr, Phe, Arg, His, Asp, Asn, Glu, Gln, Pro, Trp, Tyr, or Cys, at position 409 and said second CH3 region comprises a Lys at position 409 and: a) an Ile at position 350 and a Leu at position 405, or b) a Thr at position 370 and a Leu at position 405.

The bispecific antibody disclosed herein may be an antibody, wherein said first CH3 region comprises an Arg at position 409 and said second CH3 region comprises a Lys at position 409 and: a) an Ile at position 350 and a Leu at position 405, or b) a Thr at position 370 and a Leu at position 405.

The bispecific antibody disclosed herein may be an antibody, wherein said first CH3 region comprises a Thr at position 350, a Lys at position 370, a Phe at position 405 and an Arg at position 409 and said second CH3 region comprises a Lys at position 409 and: a) an Ile at position 350 and a Leu at position 405, or b) a Thr at position 370 and a Leu at position 405.

The bispecific antibody disclosed herein may be an antibody, wherein said first CH3 region comprises a Thr at position 350, a Lys at position 370, a Phe at position 405 and an Arg at position 409 and said second CH3 region comprises an Ile at position 350, a Thr at position 370, a Leu at position 405 and a Lys at position 409.

The bispecific antibody disclosed herein may be an antibody, wherein said first CH3 region has an amino acid other than Lys, Leu or Met at position 409 and said second CH3 region has an amino acid other than Phe at position 405, such as other than Phe, Arg or Gly at position 405; or said first CH3 region has an amino acid other than Lys, Leu or Met at position 409 and said second CH3 region has an amino acid other than Tyr, Asp, Glu, Phe, Lys, Gln, Arg, Ser or Thr at position 407.

The bispecific antibody disclosed herein may comprise a first CH3 region having an amino acid other than Lys, Leu or Met at position 409 and a second CH3 region having an amino acid other than Tyr, Asp, Glu, Phe, Lys, Gln, Arg, Ser or Thr at position 407.

The bispecific antibody disclosed herein may comprise a first CH3 region having a Tyr at position 407 and an amino acid other than Lys, Leu or Met at position 409 and a second CH3 region having an amino acid other than Tyr, Asp, Glu, Phe, Lys, Gln, Arg, Ser or Thr at position 407 and a Lys at position 409.

The bispecific antibody disclosed herein may comprise a first CH3 region having a Tyr at position 407 and an Arg at position 409 and a second CH3 region having an amino acid other than Tyr, Asp, Glu, Phe, Lys, Gln, Arg, Ser or Thr at position 407 and a Lys at position 409.

The said first CH3 region may have an amino acid other than Lys, Leu or Met, e.g. Gly, Ala, Val, Ile, Ser, Thr, Phe, Arg, His, Asp, Asn, Glu, Gln, Pro, Trp, Tyr, or Cys, at position 409 and said second CH3 region may have an amino acid other than Tyr, Asp, Glu, Phe, Lys, Gln, Arg, Ser or Thr, e.g. Leu, Met, Gly, Ala, Val, Ile, His, Asn, Pro, Trp, or Cys, at position 407. The said first CH3 region may have an amino acid other than Lys, Leu or Met, e.g. Gly, Ala, Val, Ile, Ser, Thr, Phe, Arg, His, Asp, Asn, Glu, Gln, Pro, Trp, Tyr, or Cys, at position 409 and said second CH3 region may have an Ala, Gly, His, Ile, Leu, Met, Asn, Val or Trp at position 407.

The bispecific antibody disclosed herein may be an antibody, wherein said first CH3 region has an amino acid other than Lys, Leu or Met, e.g. Gly, Ala, Val, Ile, Ser, Thr, Phe, Arg, His, Asp, Asn, Glu, Gln, Pro, Trp, Tyr, or Cys, at position 409 and said second CH3 region has a Gly, Leu, Met, Asn or Trp at position 407.

The bispecific antibody disclosed herein may e an antibody, wherein said first CH3 region has a Tyr at position 407 and an amino acid other than Lys, Leu or Met, e.g. Gly, Ala, Val, Ile, Ser, Thr, Phe, Arg, His, Asp, Asn, Glu, Gln, Pro, Trp, Tyr, or Cys, at position 409 and said second CH3 region has an amino acid other than Tyr, Asp, Glu, Phe, Lys, Gln, Arg, Ser or Thr, e.g. Leu, Met, Gly, Ala, Val, Ile, His, Asn, Pro, Trp, or Cys, at position 407 and a Lys at position 409.

The bispecific antibody disclosed herein may be an antibody, wherein said first CH3 region has a Tyr at position 407 and an amino acid other than Lys, Leu or Met, e.g. Gly, Ala, Val, Ile, Ser, Thr, Phe, Arg, His, Asp, Asn, Glu, Gln, Pro, Trp, Tyr, or Cys, at position 409 and said second CH3 region has an Ala, Gly, His, Ile, Leu, Met, Asn, Val or Trp at position 407 and a Lys at position 409.

The bispecific antibody disclosed herein may be an antibody, wherein said first CH3 region has a Tyr at position 407 and an amino acid other than Lys, Leu or Met, e.g. Gly, Ala, Val, Ile, Ser, Thr, Phe, Arg, His, Asp, Asn, Glu, Gln, Pro, Trp, Tyr, or Cys, at position 409 and said second CH3 region has a Gly, Leu, Met, Asn or Trp at position 407 and a Lys at position 409.

The bispecific antibody disclosed herein may be an antibody, wherein said first CH3 region has a Tyr at position 407 and an Arg at position 409 and said second CH3 region has an amino acid other than Tyr, Asp, Glu, Phe, Lys, Gln, Arg, Ser or Thr, e.g. Leu, Met, Gly, Ala, Val, Ile, His, Asn, Pro, Trp, or Cys, at position 407 and a Lys at position 409.

The bispecific antibody disclosed herein may be an antibody, wherein said first CH3 region has a Tyr at position 407 and an Arg at position 409 and said second CH3 region has an Ala, Gly, His, Ile, Leu, Met, Asn, Val or Trp at position 407 and a Lys at position 409.

The bispecific antibody disclosed herein may be an antibody, wherein said first CH3 region has a Tyr at position 407 and an Arg at position 409 and said second CH3 region has a Gly, Leu, Met, Asn or Trp at position 407 and a Lys at position 409.

The bispecific antibody disclosed herein may be an antibody, wherein the first CH3 region has an amino acid other than Lys, Leu or Met, e.g. Gly, Ala, Val, Ile, Ser, Thr, Phe, Arg, His, Asp, Asn, Glu, Gln, Pro, Trp, Tyr, or Cys, at position 409, and the second CH3 region may have
(i) an amino acid other than Phe, Leu and Met, e.g. Gly, Ala, Val, Ile, Ser, Thr, Lys, Arg, His, Asp, Asn, Glu, Gln, Pro, Trp, Tyr, or Cys, at position 368, or
(ii) a Trp at position 370, or
(iii) an amino acid other than Asp, Cys, Pro, Glu or Gln, e.g. Phe, Leu, Met, Gly, Ala, Val, Ile, Ser, Thr, Lys, Arg, His, Asn, Trp, Tyr, or Cys, at position 399 or
(iv) an amino acid other than Lys, Arg, Ser, Thr, or Trp, e.g. Phe, Leu, Met, Ala, Val, Gly, Ile, Asn, His, Asp, Glu, Gln, Pro, Tyr, or Cys, at position 366.

The first CH3 region may have an Arg, Ala, His or Gly at position 409, and the second CH3 region may have
(i) a Lys, Gln, Ala, Asp, Glu, Gly, His, Ile, Asn, Arg, Ser, Thr, Val, or Trp at position 368, or
(ii) a Trp at position 370, or
(iii) an Ala, Gly, Ile, Leu, Met, Asn, Ser, Thr, Trp, Phe, His, Lys, Arg or Tyr at position 399, or
(iv) an Ala, Asp, Glu, His, Asn, Val, Gln, Phe, Gly, Ile, Leu, Met, or Tyr at position 366.

The first CH3 region may have an Arg at position 409, and the second CH3 region may have
(i) an Asp, Glu, Gly, Asn, Arg, Ser, Thr, Val, or Trp at position 368, or
(ii) a Trp at position 370, or
(iii) a Phe, His, Lys, Arg or Tyr at position 399, or
(iv) an Ala, Asp, Glu, His, Asn, Val, Gln at position 366.

The bispecific antibody may comprise a first and second heavy chain, wherein each of said first and second heavy chains comprises at least a hinge region, a CH2 and a CH3 region, wherein (i) the amino acid in the position corresponding to F405 in human IgG1 heavy chain is L in said first heavy chain, and the amino acid in the position corresponding to K409 in human IgG1 heavy chain is R in said second heavy chain, or (ii) the amino acid in the position corresponding to K409 in human IgG1 heavy chain is R in said first heavy chain, and the amino acid in the position corresponding to F405 in human IgG1 heavy chain is L in said second heavy chain.

In addition to the above-specified amino-acid substitutions, said first and second heavy chains may contain further amino-acid substitutions, deletion or insertions relative to wild-type heavy chain sequences.

In one embodiment of the present disclosure neither said first nor said second Fc sequence comprises a Cys-Pro-Ser-Cys sequence in the (core) hinge region. In an alternative embodiment both said first and said second Fc sequence comprise a Cys-Pro-Pro-Cys sequence in the (core) hinge region

Preferably the antibody comprised in the pharmaceutical formulation of the invention induces Fc-mediated effector function to a lesser extent compared to another antibody comprising the same first and second antigen binding regions and two heavy chain constant regions (CHs) comprising human IgG1 hinge, CH2 and CH3 regions.

The said first and second heavy chain constant regions (CHs) may be modified so that the antibody induces Fc-mediated effector function to a lesser extent compared to an antibody which is identical except for comprising non-modified first and second heavy chain constant regions (CHs).

The said Fc-mediated effector function is preferably measured by binding to Fcγ receptors, binding to C1q, or induction of Fc-mediated cross-linking of Fcγ receptors.

In particular, the Fc-mediated effector function is measured by binding to C1q.

The said first and second heavy chain constant regions may have been modified so that binding of C1q to said antibody is reduced compared to a wild-type antibody, preferably reduced by at least 70%, at least 80%, at least 90%, at least 95%, at least 97%, or 100%, wherein C1q binding is preferably determined by ELISA.

The binding agent comprised by the pharmaceutical formulation may be one wherein in at least one of said first and second heavy chain constant region (CH) one or more amino acids in the positions corresponding to positions L234, L235, D265, N297, and P331 in a human IgG1 heavy chain according to EU numbering, are not L, L, D, N, and P, respectively.

In the binding agent of the pharmaceutical formulation, the positions corresponding to positions L234 and L235 in a human IgG1 heavy chain according to EU numbering may be F and E, respectively, in said first and second heavy chains.

In the binding agent of the pharmaceutical formulation the positions corresponding to positions L234, L235, and D265 in a human IgG1 heavy chain according to EU numbering may be F, E, and A, respectively, in said first and second heavy chain constant regions (HCs).

The pharmaceutical formulation may comprise a binding agent, wherein the positions corresponding to positions L234, L235, and D265 in a human IgG1 heavy chain according to EU numbering of both the first and second heavy chain constant regions are F, E, and A, respectively, and wherein (i) the position corresponding to F405 in a human IgG1 heavy chain according to EU numbering of the first heavy chain constant region is L, and the position corresponding to K409 in a human IgG1 heavy chain according to EU numbering of the second heavy chain constant region is R, or (ii) the position corresponding to K409 in a human IgG1 heavy chain according to EU numbering of the first heavy chain is R, and the position corresponding to F405 in a human IgG1 heavy chain according to EU numbering of the second heavy chain is L.

The pharmaceutical formulation may comprise a binding agent, wherein the positions corresponding to positions L234 and L235 in a human IgG1 heavy chain according to EU numbering of both the first and second heavy chain constant regions are F and E, respectively, and wherein (i) the position corresponding to F405 in a human IgG1 heavy chain according to EU numbering of the first heavy chain constant region is L, and the position corresponding to K409 in a human IgG1 heavy chain according to EU numbering of the second heavy chain is R, or (ii) the position corresponding to K409 in a human IgG1 heavy chain according to EU numbering of the first heavy chain constant region is R, and the position corresponding to F405 in a human IgG1 heavy chain according to EU numbering of the second heavy chain is L.

In particular embodiments, the first binding arm may comprises a kappa (κ) light chain, such as a kappa light chain comprising the amino acid sequence set forth in SEQ ID NO: 26 and said second binding arm comprises a lambda (λ) light chain, such as a lambda light chain comprising the amino acid sequence set forth in SEQ ID NO: 27.

In other embodiments, the first binding arm comprises a lambda (λ) light chain, such as a lambda light chain comprising the amino acid sequence set forth in SEQ ID NO: 27 and said second binding arm comprises a kappa (κ) light chain, such as a kappa light chain comprising the amino acid sequence set forth in SEQ ID NO: 26.

In still other embodiments both the first binding arm and the second binding arm comprises a lambda (λ) light chain, such as a lambda light chain comprising the amino acid sequence set forth in SEQ ID NO: 27.

In further embodiments, both the first binding arm and the second binding arm comprises a kappa (κ) light chain, such as a kappa light chain comprising the amino acid sequence set forth in SEQ ID NO: 26.

The binding agent may be one wherein the first binding arm comprises the amino acid sequences set forth in SEQ ID NO: 24 and the second binding arm comprises the amino acid sequence set forth in SEQ ID NO: 25.

Alternatively, the binding agent is one wherein the first binding arm comprises the amino acid sequences set forth in SEQ ID NO: 25 and the second binding arm comprises the amino acid sequence set forth in SEQ ID NO: 24.

The binding agent may be one that induces and/or enhances proliferation of T cells.

In particular, the said T cells may be CD⁴⁺ and/or CD⁸⁺ T cells.

In the pharmaceutical formulation according to the invention the binding agent may be one which activates CD137 signaling only when the second antigen-binding region binds to PD-L1.

Proliferation of T cells may be measured by co-culturing T-cells expressing a specific T-cell receptor (TCR) with dendritic cells (DCs) presenting the corresponding antigen on the major histocompatibility complex, which is recognized by the TCR.

In one embodiment, said induction or enhancement of proliferation of T cells is determined by an antigen-specific assay, where DCs are transfected with claudin-6 antigen and T cells are transfected with a TCR that recognizes a claudin-6-derived epitope presented in HLA-A2 on the DC. This assay is described in Example 7.

The binding agent of the invention may be able to mediate expansion of tumor-infiltrating lymphocytes (TILs) in an ex vivo culture of human tumor tissue. The expansion of TILs may be 1.5 fold or more, 2-fold or more, 3-fold or more, 4-fold or more, 5-fold or more, 6-fold or more, 7-fold or more, 8-fold or more, 9 fold or more or 10-fold or more. The expansion of CD3⁻CD56⁺ natural killer (NK) cells may be from at least 10-fold, such as at least 20-fold, at least 30-fold, at least 40-fold, or such as at least 50-fold. The expansion of CD3⁺CD8⁺ cytotoxic T-lymphocytes (CTLs) may be at least 2-fold, at least 3-fold, at least 4-fold, at least 5-fold, at least 6-fold or such as at least 7-fold. Preferably, the expansion of TILs is determined as TIL expansion from a human non-small-cell lung carcinoma tissue specimen in response to incubation with a concentration of bispecific binding agent corresponding to 0.01, 0.1 and 1 μg/mL, such as in response to incubation with a concentration of bispecific binding agent corresponding to 0.1 μg/mL.

The expansion of TILs may be determined in an assay comprising the steps of:
i) providing a resection specimen, such as a fresh resection specimen, of tumor tissue and washing the specimen in hematopoietic cell medium,
ii) cutting the tumor tissue in pieces having a diameter of 1-2 mm and providing a sample containing two pieces of tumor tissue,
iii) incubating the sample with bispecific binding agent of the invention at a concentration of 0.1 μg/ml in hematopoietic cell medium, such as Lonza™ X-VIVO™ 15, with 10% Human Serum Albumin, antibiotics and Proleukin®S (recombinant human IL-2 analog; SEQ ID NO: 56) in a well of a tissue culture plate at 37° C., 5% CO₂ for 72 hours; wherein when more than 25 TIL microclusters are observed in a sample, then the cells in said sample are split and transferred into 6 samples, or 6 wells in a tissue culture plate,
iv) harvesting TILs after a total incubation period of 10-14 days and subjecting them to staining with labelled antibodies against human CD3, human CD4, human CD56 and human CD8 and with a dye that stains non-viable cells, such as aminoactinomycin D; and
v) analyzing each sample by flow cytometry.

The binding agent of the invention may in particular be able to induce expansion of CD40+ and CD⁸⁺ T-cells in a population of peripheral blood mononuclear cells (PBMCs), wherein T-cells are activated, such as sub-optimally activated, by incubation with a an anti-CD3 antibody, such as clone UCHT1), preferably at a concentration between 0.03 and 0.1 μg/mL and are preferably incubated with bispecific binding agent according to the invention at a concentration corresponding to 0.2 μg/mL. In particular, the process for determining T-cell expansion may comprise the steps of:
i) obtaining PBMCs from buffy coats of healthy donors, such as by isolation of a Ficoll gradient,
ii) labelling the PBMCs with carboxyfluorescein succinimidyl ester (CFSE) in PBS,
iii) providing a sample comprising 75000 CFSE-labelled PBMCs and incubating the sample with anti-CD3 antibody, preferably at a concentration between 0.03 and 0.1 μg/mL as predetermined for each donor to be a concentration inducing suboptimal T-cell proliferation, and with bispecific binding agent of the invention at a concentration of 0.2 μg/mL, at 37° C., 5% CO₂, for four days in Iscove's Modified Dulbecco's Medium with glutamine and supplemented with human AB serum,
iv) subjecting the PBMCs to staining with labeled antibodies against human CD4, human CD8, human CD56 and with with a dye that stains non-viable cells, such as 7-aminoactinomycin D; and
v) analyzing CSFE in different subpopulations (CD⁴⁺ and CD⁸⁺ T-cells) in the samples by flow cytometry.

Traditional methods such as the hybrid hybridoma and chemical conjugation methods (Marvin and Zhu (2005) Acta Pharmacol Sin 26:649) can be used in the preparation of the binding agent disclosed in the context of the invention. Co-expression in a host cell of two antibodies, consisting of different heavy and light chains, leads to a mixture of possible antibody products in addition to the desired bispecific binding agent, which can then be isolated by, e.g., affinity chromatography or similar methods.

Strategies favoring the formation of a functional bispecific, product, upon co-expression of different antibody constructs can also be used, e.g., the method described by Lindhofer et al. (1995 J Immunol 155:219). Fusion of rat and mouse hybridomas producing different antibodies leads to a limited number of heterodimeric proteins because of preferential species-restricted heavy/light chain pairing. Another strategy to promote formation of heterodimers over homodimers is a “knob-into-hole” strategy in which a protuberance is introduced on a first heavy-chain polypeptide and a corresponding cavity in a second heavy-chain polypeptide, such that the protuberance can be positioned in the cavity at the interface of these two heavy chains so as to promote heterodimer formation and hinder homodimer formation. “Protuberances” are constructed by replacing small amino-acid side-chains from the interface of the first polypeptide with larger side chains. Compensatory “cavities” of identical or similar size to the protuberances are created in the interface of the second polypeptide by replacing large amino-acid side-chains with smaller ones (U.S. Pat. No. 5,731,168). EP1870459 (Chugai) and WO2009089004 (Amgen) describe other strategies for favoring heterodimer formation upon co-expression of different antibody domains in a host cell. In these methods, one or more residues that make up the CH3-CH3 interface in both CH3 domains are replaced with a charged amino acid such that homodimer formation is electrostatically unfavorable and heterodimerization is electrostatically favorable. WO2007110205 (Merck) describe yet another strategy, wherein differences between IgA and IgG CH3 domains are exploited to promote heterodimerization.

Another in vitro method for producing bispecific antibodies has been described in WO2008119353 (Genmab), wherein a bispecific antibody is formed by “Fab-arm” or “half-molecule” exchange (swapping of a heavy chain and attached light chain) between two monospecific IgG4- or IgG4-like antibodies upon incubation under reducing conditions. The resulting product is a bispecific antibody having two Fab arms which may comprise different sequences.

A preferred method for preparing bispecific PD-L1xCD137 binding agents as disclosed herein includes the methods described in WO2011131746 and WO2013060867 (Genmab) comprising the following steps:
a) providing a first antibody comprising an Fc region, said Fc region comprising a first CH3 region;
b) providing a second antibody comprising a second Fc region, said Fc region comprising a second CH3 region, wherein the first antibody is a CD137 antibody and the second antibody is a PD-L1 antibody, or vice versa;
   wherein the sequences of said first and second CH3 regions are different and are such that the heterodimeric interaction between said first and second CH3 regions is stronger than each of the homodimeric interactions of said first and second CH3 regions;
c) incubating said first antibody together with said second antibody under reducing conditions; and
d) obtaining said bispecific PD-L1xCD137 antibody.

Similarly, there is provided a method for producing a binding agent as disclosed in the context of the invention, comprising the steps of:
a) culturing a host cell producing a first antibody comprising an antigen-binding region capable of binding to human CD137 as defined herein and purifying said first antibody from the culture;
b) culturing a host cell producing a second antibody comprising an antigen-binding region capable of binding to human PD-L1 as defined herein purifying said second antibody from the culture;
c) incubating said first antibody together with said second antibody under reducing conditions sufficient to allow the cysteines in the hinge region to undergo disulfide-bond isomerization, and
d) obtaining said bispecific antibody.

In one embodiment of the invention, the said first antibody together with said second antibody are incubated under reducing conditions sufficient to allow the cysteines in the hinge region to undergo disulfide-bond isomerization, wherein the heterodimeric interaction between said first and second antibodies in the resulting heterodimeric antibody is such that no Fab-arm exchange occurs at 0.5 mM GSH after 24 hours at 37° C.

Without being limited to theory, in step c), the heavy-chain disulfide bonds in the hinge regions of the parent antibodies are reduced and the resulting cysteines are then able to form inter heavy-chain disulfide bonds with cysteine residues of another parent antibody molecule (originally with a different specificity). In one embodiment of this method, the reducing conditions in step c) comprise the addition of a reducing agent, e.g. a reducing agent selected from the group consisting of: 2-mercaptoethylamine (2-MEA), dithiothreitol (DTT), dithioerythritol (DTE), glutathione, tris(2-carboxyethyl)phosphine (TCEP), L-cysteine and beta-mercapto-ethanol, preferably a reducing agent selected from the group consisting of: 2-mercaptoethylamine, dithiothreitol and tris(2-carboxyethyl)phosphine. In a further embodiment, step c) comprises restoring the conditions to become non-reducing or less reducing, for example by removal of a reducing agent, e.g. by desalting.

For this method, any of the CD137 and PD-L1 antibodies described above may be used including first and second CD137 and PD-L1 antibodies, respectively, comprising a first and/or second Fc region. Examples of such first and second Fc regions, including combination of such first and second Fc regions may include any of those described above. In a particular embodiment, the first and second CD137 and PD-L1 antibodies, respectively, may be chosen so as to obtain a bispecific antibody as described herein.

In one embodiment of this method, said first and/or second antibodies are full-length antibodies.

The Fc regions of the first and second antibodies may be of any isotype, including, but not limited to, IgG1, IgG2, IgG3 or IgG4. Preferably, the Fc regions of both said first and said second antibodies are of the IgG1 isotype. Alternatively, one of the Fc regions of said antibodies is of the IgG1 isotype and the other of the IgG4 isotype. In the latter case, the resulting bispecific antibody comprises an Fc sequence of an IgG1 and an Fc sequence of IgG4 and may thus have interesting intermediate properties with respect to activation of effector functions.

One of the antibody starting proteins may have been engineered to not bind Protein A, thus allowing to separate the heterodimeric protein from said homodimeric starting protein by passing the product over a protein A column.

As described above, the sequences of the first and second CH3 regions of the homodimeric starting antibodies are different and are such that the heterodimeric interaction between said first and second CH3 regions is stronger than each of the homodimeric interactions of said first and second CH3 regions. More details on these interactions and how they can be achieved are provided in WO2011131746 and WO2013060867 (Genmab), which are hereby incorporated by reference in their entirety.

In particular, a stable bispecific PD-L1xCD137 antibody can be obtained at high yield using the above method of the invention on the basis of two homodimeric starting antibodies which bind CD137 and PD-L1, respectively, and contain only a few, conservative, asymmetrical mutations in the CH3 regions. Asymmetrical mutations mean that the sequences of said first and second CH3 regions contain amino acid substitutions at non-identical positions.

The bispecific antibodies disclosed herein may also be obtained by co-expression of constructs encoding the first and second polypeptides in a single cell. Thus, in a further aspect, the invention relates to a method for producing a bispecific antibody, said method comprising the following steps:
a) providing a first nucleic-acid construct encoding a first polypeptide comprising a first Fc sequence and a first antigen-binding region of a first antibody heavy chain, said first Fc sequence comprising a first CH3 region,
b) providing a second nucleic-acid construct encoding a second polypeptide comprising a second Fc sequence and a second antigen-binding region of a second antibody heavy chain, said second Fc sequence comprising a second CH3 region,
   wherein the sequences of said first and second CH3 regions are different and are such that the heterodimeric interaction between said first and second CH3 regions is stronger than each of the homodimeric interactions of said first and second CH3 regions, and wherein said first homodimeric protein has an amino acid other than Lys, Leu or Met at position 409 and said second homodimeric protein has an amino-acid substitution at a position selected from the group consisting of: 366, 368, 370, 399, 405 and 407, optionally wherein said first and second nucleic acid constructs encode light chain sequences of said first and second antibodies
c) co-expressing said first and second nucleic-acid constructs in a host cell, and
d) obtaining said heterodimeric protein from the cell culture.

The pharmaceutical formulation provided according to the invention is preferably an aqueous formulation.

The invention also provides a method for producing a pharmaceutical formulation as defined above, the method comprising providing a binding agent as defined above and combining it with:
a. a histidine buffer,
b. about 100 to about 400 mM of a sugar, and
c. about 0.001 to about 0.1% (w/v) non-ionic surfactant;
   at a pH between about 4.5 and about 6.5.

It will be understood that the particulars provided above concerning the histidine buffer, the sugar, the non-ionic surfactant and the pH also apply to the method for producing the formulation.

In a further aspect, the present invention provides a pharmaceutical formulation as defined above for use as a medicament.

The pharmaceutical formulation may in particular be for use in the treatment of cancer.

The invention further provides a method for treatment of a disease comprising administering an effective amount of a pharmaceutical formulation as defined herein to a subject in need thereof.

The disease may in particular be cancer.

The present invention also provides a method of inducing cell death or inhibiting growth and/or proliferation of a tumor cell expressing PD-L1 comprising administering an effective amount of a pharmaceutical formulation as defined above to a subject in need thereof and/or bearing said tumor cell.

In relation to the pharmaceutical formulation for use as set forth above or the method defined above, the cancer may in particular be characterized by the presence of solid tumors or may be selected from the group consisting of: melanoma, ovarian cancer, lung cancer, colorectal cancer, head and neck cancer, gastric cancer, breast cancer, renal cancer, bladder cancer, esophageal cancer, pancreatic cancer, hepatic cancer, thymoma and thymic carcinoma, brain cancer, glioma, adrenocortical carcinoma, thyroid cancer, other skin cancers, sarcoma, multiple myeloma, leukemia, lymphoma, myelodysplastic syndromes, ovarian cancer, endometriosis cancer, prostate cancer, penile cancer, Hodgkin's lymphoma, non-Hodgkin's lymphoma, Merkel cell carcinoma and mesothelioma.

The cancer may in particular be non-small cell lung cancer (NSCLC).

The invention further provides the use of a pharmaceutical formulation as defined above, for the manufacture of a medicament, such as a medicament for the treatment of cancer, e.g. a cancer characterized by the presence of solid tumors or a cancer selected from the group consisting of: melanoma, ovarian cancer, lung cancer, colon cancer and head and neck cancer.

The lung cancer may in particular be non-small cell lung cancer (NSCLC).

Dosage regimens in the above methods of treatment and uses are adjusted to provide the optimum desired response (e.g., a therapeutic response). For example, a single bolus may be administered, several divided doses may be administered over time or the dose may be proportionally reduced or increased as indicated by the exigencies of the therapeutic situation. Parenteral compositions may be formulated in dosage unit form for ease of administration and uniformity of dosage.

The efficient dosages and the dosage regimens for the pharmaceutical formulation depend on the disease or condition to be treated and may be determined by the persons skilled in the art. An exemplary, non-limiting range for a therapeutically effective amount of a compound of the present invention is about 0.001-10 mg/kg, such as about 0.001-5 mg/kg, for example about 0.001-2 mg/kg, such as about 0.001-1 mg/kg, for instance about 0.001, about 0.01, about 0.1, about 1 or about 10 mg/kg. Another exemplary, non-limiting range for a therapeutically effective amount of a binding agent (e.g. a bispecific antibody) of the present invention is about 0.1-100 mg/kg, such as about 0.1-50 mg/kg, for example about 0.1-20 mg/kg, such as about 0.1-10 mg/kg, for instance about 0.5, about such as 0.3, about 1, about 3, about 5, or about 8 mg/kg.

A physician or veterinarian having ordinary skill in the art may readily determine and prescribe the effective amount of the pharmaceutical formulation required. For example, the physician or veterinarian could start doses of the binding agent (e.g. a bispecific antibody) employed in the pharmaceutical composition at levels lower than that required to achieve the desired therapeutic effect and gradually increase the dosage until the desired effect is achieved. In general, a suitable daily dose of a binding agent (e.g. a bispecific antibody) of the present invention will be that amount of the compound which is the lowest dose effective to produce a therapeutic effect. Administration may e.g. be parenteral, such as intravenous, intramuscular or subcutaneous. In one embodiment, the binding agents (e.g. bispecific antibodies) may be administered by infusion in a weekly dosage of calculated by mg/m². Such dosages can, for example, be based on the mg/kg dosages provided above according to the following: dose (mg/kg)×70: 1.8. Such administration may be repeated, e.g., 1 to 8 times, such as 3 to 5 times. The administration may be performed by continuous infusion over a period of from 2 to 24 hours, such as from 2 to 12 hours. In one embodiment, the binding agents (e.g. bispecific antibodies) may be administered by slow continuous infusion over a long period, such as more than 24 hours, to reduce toxic side effects.

The pharmaceutical formulation may be administered in a weekly dosage of calculated as a fixed dose for up to 8 times, such as from 4 to 6 times when given once a week. Such regimen may be repeated one or more times as necessary, for example, after 6 months or 12 months. Such fixed dosages can, for example, be based on the mg/kg dosages provided above, with a body weight estimate of 70 kg. The dosage may be determined or adjusted by measuring the amount of binding agent (e.g. bispecific antibody) of the present invention in the blood upon administration by for instance taking out a biological sample and using anti-idiotypic antibodies which target the PD-L1 antigen antigen-binding region of the antibodies of the present invention.

The pharmaceutical formulation may be administered as maintenance therapy, such as, e.g., once a week for a period of 6 months or more.

The pharmaceutical formulation may also be administered prophylactically to reduce the risk of developing cancer, delay the onset of the occurrence of an event in cancer progression, and/or reduce the risk of recurrence when a cancer is in remission.

When used as defined above the pharmaceutical formulation according to the invention is preferably administered intravenously.

The use or method defined above may comprise using the pharmaceutical formulation in combination with one or more further therapeutic agents, such as a chemotherapeutic agent.

The present invention is further illustrated by the following examples, which should not be construed as limiting the scope of the invention.

### SEQUENCES

TABLE 1

| SEQ | | |
|---|---|---|
| ID | NAME | SEQUENCE |
| 1 | VH_b12 | QVQLVQSGAEVKKPGASVKVSCQASGYRFSNFVIHWVRQAPGQ |
| | | RFEWMGWINPYNGNKEFSAKFQDRVTFTADTSANTAYMELRSL |
| | | RSADTAVYYCARVGPYSWDDSPQDNYYMDVWGKGTTVIVSS |
| 2 | VH_b12-CDR1 | GYRFSNFV |
| 3 | VH_b12-CDR2 | INPYNGNK |
| 4 | VH_b12-CDR3 | ARVGPYSWDDSPQDNYYMDV |
| 5 | VL_b12 | EIVLTQSPGTLSLSPGERATFSCRSSHSIRSRRVAWYQHKPGQA |
| | | PRLVIHGVSNRASGISDRFSGSGSGTDFTLTITRVEPEDFALYYC |
| | | QVYGASSYTFGQGTKLERK |
| 6 | VL_b12-CDR1 | HSIRSRR |
| | VL_b12-CDR2 | GVS |
| 7 | VL_b12-CDR3 | QVYGASSYT |
| 8 | CD137-009 | QSLEESGGRLVTPGTPLTLTCTVSGFSLNDYWMSWVRQAPGKG |
| | | LEWIGYIDVGGSLYYASWAKGRFTISRTSTTVDLKMTSLTTEDTA |
| | | TYFCARGGLTYGFDLWGPGTLVTVSS |
| 9 | VH_CD137-009_CDR1 | GFSLNDYW |
| 10 | VH_CD137-009_CDR2 | IDVGGSL |
| 11 | VH_CD137-009_CDR3 | ARGGLTYGFDL |
| 12 | VL_CD137-009 | DIVMTQTPASVSEPVGGTVTINCQASEDISSYLAWYQQKPGQRP |
| | | KRLIYGASDLASGVPSRFSASGSGTEYALTISDLESADAATYYCH |
| | | YYATISGLGVAFGGGTEVVVK |
| 13 | VL_CD137-009_CDR1 | EDISSY |
| | VL_CD137-009_CDR2 | GAS |
| 14 | VL_CD137-009_CDR3 | HYYATISGLGVA |
| 15 | VH_CD137-009-H7 | EVQLVESGGGLVQPGRSLRLSCTASGFSLNDYWMSWVRQAPG |
| | | KGLEWVGYIDVGGSLYYAASVKGRFTISRDDSKSIAYLQMNSLK |
| | | TEDTAVYYCARGGLTYGFDLWGQGTLVTVSS |
| 9 | VH_CD137-009- | GFSLNDYW |
| | H7_CDR1 | |
| 10 | VH_CD137-009- | IDVGGSL |
| | H7_CDR2 | |
| 11 | VH_CD137-009- | ARGGLTYGFDL |
| | H7_CDR3 | |
| 16 | VL_CD137-009-L2 | DIVMTQSPSSLSASVGDRVTITCQASEDISSYLAWYQQKPGKAP |
| | | KRLIYGASDLASGVPSRFSASGSGTDYTFTISSLQPEDIATYYCH |
| | | YYATISGLGVAFGGGTKVEIK |
| 13 | VL_CD137-009- | EDISSY |
| | L2_CDR1 | |
| | VL_CD137-009- | GAS |
| | L2_CDR2 | |
| 14 | VL_CD137-009- | HYYATISGLGVA |
| | L2_CDR3 | |
| 17 | VH-PD-L1-547 | EVQLLEPGGGLVQPGGSLRLSCEASGSTFSTYAMSWVRQAPGK |
| | | GLEWVSGFSGSGGFTFYADSVRGRFTISRDSSKNTLFLQMSSLR |
| | | AEDTAVYYCAIPARGYNYGSFQHWGQGTLVTVSS |
| 18 | VH-PD-L1-547-CDR1 | GSTFSTYA |
| 19 | VH-PD-L1-547-CDR2 | FSGSGGFT |
| 20 | VH-PD-L1-547-CDR3 | AIPARGYNYGSFQH |
| 21 | VL-PD-L1-547 | SYVLTQPPSVSVAPGQTARITCGGNNIGSKSVHWYQQKPGQAP |
| | | VLVVYDDNDRPSGLPERFSGSNSGNTATLTISRVEAGDEADYYC |
| | | QVWDSSSDHVVFGGGTKLTVL |
| 22 | VL-PD-L1-547-CDR1 | NIGSKS |
| | VL-PD-L1-547-CDR2 | DDN |
| 23 | VL-PD-L1-547-CDR3 | QVWDSSSDHVV |
| 24 | IgG1-FEAR-Fc | ASTKGPSVFPLAPSSKSTSGGTAALGCLVKDYFPEPVTVSWNSG |
| | | ALTSGVHTFPAVLQSSGLYSLSSVVTVPSSSLGTQTYICNVNHKP |
| | | SNTKVDKRVEPKSCDKTHTCPPCPAPEFEGGPSVFLFPPKPKDTL |
| | | MISRTPEVTCVVVAVSHEDPEVKFNWYVDGVEVHNAKTKPREE |
| | | QYNSTYRVVSVLTVLHQDWLNGKEYKCKVSNKALPAPIEKTISK |
| | | AKGQPREPQVYTLPPSREEMTKNQVSLTCLVKGFYPSDIAVEWE |
| | | SNGQPENNYKTTPPVLDSDGSFFLYSRLTVDKSRWQQGNVFSC |
| | | SVMHEALHNHYTQKSLSLSPGK |
| 25 | IgG1-FEAL-Fc | ASTKGPSVFPLAPSSKSTSGGTAALGCLVKDYFPEPVTVSWNSG |
| | | ALTSGVHTFPAVLQSSGLYSLSSVVTVPSSSLGTQTYICNVNHKP |
| | | SNTKVDKRVEPKSCDKTHTCPPCPAPEFEGGPSVFLFPPKPKDTL |
| | | MISRTPEVTCVVVAVSHEDPEVKFNWYVDGVEVHNAKTKPREE |
| | | QYNSTYRVVSVLTVLHQDWLNGKEYKCKVSNKALPAPIEKTISK |
| | | AKGQPREPQVYTLPPSREEMTKNQVSLTCLVKGFYPSDIAVEWE |
| | | SNGQPENNYKTTPPVLDSDGSFLLYSKLTVDKSRWQQGNVFSC |
| | | SVMHEALHNHYTQKSLSLSPGK |
| 26 | Kappa-C | RTVAAPSVFIFPPSDEQLKSGTASVVCLLNNFYPREAKVQWKVD |
| | | NALQSGNSQESVTEQDSKDSTYSLSSTLTLSKADYEKHKVYACE |
| | | VTHQGLSSPVTKSFNRGEC |
| 27 | Lambda-C | GQPKAAPSVTLFPPSSEELQANKATLVCLISDFYPGAVTVAWKAD |
| | | SSPVKAGVETTTPSKQSNNKYAASSYLSLTPEQWKSHRSYSCQV |
| | | THEGSTVEKTVAPTECS |
| 28 | HumanPD-L1 | MRIFAVFIFMTYWHLLNAFTVTVPKDLYVVEYGSNMTIECKFPVE |
| | | KQLDLAALIVYWEMEDKNIIQFVHGEEDLKVQHSSYRQRARLLK |
| | | DQLSLGNAALQITDVKLQDAGVYRCMISYGGADYKRITVKVNAP |
| | | YNKINQRILVVDPVTSEHELTCQAEGYPKAEVIWTSSDHQVLSG |
| | | KTTTTNSKREEKLFNVTSTLRINTTTNEIFYCTFRRLDPEENHTAE |
| | | LVIPELPLAHPPNERTHLVILGAILLCLGVALTFIFRLRKGRMMDVK |
| | | KCGIQDTNSKKQSDTHLEET |
| 29 | Cynomolgus monkey | MRIFAVFIFTIYWHLLNAFTVTVPKDLYVVEYGSNMTIECKFPVEK |
| | PD-L1 | QLDLTSLIVYWEMEDKNIIQFVHGEEDLKVQHSNYRQRAQLLKD |
| | | QLSLGNAALRITDVKLQDAGVYRCMISYGGADYKRITVKVNAPY |
| | | NKINQRILVVDPVTSEHELTCQAEGYPKAEVIWTSSDHQVLSGK |
| | | TTTTNSKREEKLLNVTSTLRINTTANEIFYCIFRRLDPEENHTAELV |
| | | IPELPLALPPNERTHLVILGAIFLLLGVALTFIFYLRKGRMMDMKKC |
| | | GIRVTNSKKQRDTQLEET |
| 30 | Human CD137 | MGNSCYNIVATLLLVLNFERTRSLQDPCSNCPAGTFCDNNRNQI |
| | | CSPCPPNSFSSAGGQRTCDICRQCKGVFRTRKECSSTSNAECDC |
| | | TPGFHCLGAGCSMCEQDCKQGQELTKKGCKDCCFGTFNDQKR |
| | | GICRPWTNCSLDGKSVLVNGTKERDVVCGPSPADLSPGASSVTP |
| | | PAPAREPGHSPQIISFFLALTSTALLFLLFFLTLRFSVVKRGRKKLL |
| | | YIFKQPFMRPVQTTQEEDGCSCRFPEEEEGGCEL |
| 31 | Cynomolgus monkey | MGNSCYNIVATLLLVLNFERTRSLQDLCSNCPAGTFCDNNRSQI |
| | CD137 | CSPCPPNSFSSAGGQRTCDICRQCKGVFKTRKECSSTSNAECDC |
| | | ISGYHCLGAECSMCEQDCKQGQELTKKGCKDCCFGTFNDQKRG |
| | | ICRPWTNCSLDGKSVLVNGTKERDVVCGPSPADLSPGASSATPP |
| | | APAREPGHSPQIIFFLALTSTVVLFLLFFLVLRFSVVKRSRKKLLYIF |
| | | KQPFMRPVQTTQEEDGCSCRFPEEEEGGCEL |
| 32 | Human CD137 shuffle | MGNSCYNIVATLLLVLNFERTRSVPDPCSNCSAGTFCGKNIQELC |
| | 6 (amino acids 24-47 | MPCPPNSFSSAGGQRTCDICRQCKGVFRTRKECSSTSNAECDC |
| | of wild boar CD137) | TPGFHCLGAGCSMCEQDCKQGQELTKKGCKDCCFGTFNDQKR |
| | | GICRPWTNCSLDGKSVLVNGTKERDVVCGPSPADLSPGASSVTP |
| | | PAPAREPGHSPQIISFFLALTSTALLGGCEL |
| 33 | Human CD137 shuffle | MGNSCYNIVATLLLVLNFERTRSLQDPCSNCPAGTFCDNNRNQI |
| | 5 (amino acids 48-88 | CSPCPLNSFSSTGGQMNCDMCRKCEGVFKTKRACSPTRDAECE |
| | of African elephant | CTPGFHCLGAGCSMCEQDCKQGQELTKKGCKDCCFGTFNDQK |
| | CD137) | RGICRPWTNCSLDGKSVLVNGTKERDVVCGPSPADLSPGASSV |
| | | TPPAPAREPGHSPQIISFFLALTSTALLFLLFFLTLRFSVVKRGRKK |
| | | LLYIFKQPFMRPVQTTQEEDGCSCRFPEEEEGGCEL |
| 34 | Human CD137 shuffle | MGNSCYNIVATLLLVLNFERTRSLQDPCSNCPAGTFCDNNRNQI |
| | 4 (amino acids 89-114 | CSPCPPNSFSSAGGQRTCDICRQCKGVFRTRKECSSTSNAECDC |
| | of wild boar CD137) | VPGFRCLGAGCAMCEEYCQQGQELTQKGCKDCCFGTFNDQKR |
| | | GICRPWTNCSLDGKSVLVNGTKERDVVCGPSPADLSPGASSVTP |
| | | PAPAREPGHSPQIISFFLALTSTALLFLLFFLTLRFSVVKRGRKKLL |
| | | YIFKQPFMRPVQTTQEEDGCSCRFPEEEEGGCEL |
| 35 | Human CD137 shuffle | MGNSCYNIVATLLLVLNFERTRSLQDPCSNCPAGTFCDNNRNQI |
| | 3 (amino acids 115- | CSPCPPNSFSSAGGQRTCDICRQCKGVFRTRKECSSTSNAECDC |
| | 138 of wild boar | TPGFHCLGAGCSMCEQDCKQGQELTKEGCKDCSFGTFNDEEHG |
| | CD137) | VCRPWTDCSLDGKSVLVNGTKERDVVCGPSPADLSPGASSVTP |
| | | PAPAREPGHSPQIISFFLALTSTALLFLLFFLTLRFSVVKRGRKKLL |
| | | YIFKQPFMRPVQTTQEEDGCSCRFPEEEEGGCEL |
| 36 | Human CD137 shuffle | MGNSCYNIVATLLLVLNFERTRSLQDPCSNCPAGTFCDNNRNQI |
| | 2 (amino acids 139- | CSPCPPNSFSSAGGQRTCDICRQCKGVFRTRKECSSTSNAECDC |
| | 161 of wild boar | TPGFHCLGAGCSMCEQDCKQGQELTKKGCKDCCFGTFNDQKR |
| | CD137) | GICRPWTNCSLAGKPVLMNGTKARDVVCGPRPADLSPGASSVT |
| | | PPAPAREPGHSPQIISFFLALTSTALLFLLFFLTLRFSVVKRGRKKL |
| | | LYIFKQPFMRPVQTTQEEDGCSCRFPEEEEGGCEL |
| 37 | Human CD137 shuffle | MGNSCYNIVATLLLVLNFERTRSLQDPCSNCPAGTFCDNNRNQI |
| | 1 (amino acids 162- | CSPCPPNSFSSAGGQRTCDICRQCKGVFRTRKECSSTSNAECDC |
| | 186 of wild | TPGFHCLGAGCSMCEQDCKQGQELTKKGCKDCCFGTFNDQKR |
| | boarCD137) | GICRPWTNCSLDGKSVLVNGTKERDVVCGPSPTDFSPGTPSTTM |
| | | PVPGGEPGHTSHIISFFLALTSTALLFLLFFLTLRFSVVKRGRKKLL |
| | | YIFKQPFMRPVQTTQEEDGCSCRFPEEEE |
| | | GGCEL |
| 38 | Wild Boar CD137 | MGNGYYNIVATVLLVMNFERTRSVPDPCSNCSAGTFCGKNIQEL |
| | | CMPCPSNSFSSTSGQKACNVCRKCEGVFRTKKECSSTSNAVCE |
| | | CVPGFRCLGAGCAMCEEYCQQGQELTQEGCKDCSFGTFNDEEH |
| | | GVCRPWTDCSLAGKPVLMNGTKARDVVCGPRPTDFSPGTPSTT |
| | | MPVPGGEPGHTSHVIIFFLALMSTAVFVLVSYLALRFSVVQQGRK |
| | | KLLYIVKQPFLKPAQTVQEEDACSCRFPEEEEGECEL |
| 39 | African Elephant | MGNGYYNMVATVLLVMNFERTGAVQDSCRDCLAGTYCVKNESQ |
| | CD137 | ICSPCPLNSFSSTGGQMNCDMCRKCEGVFKTKRACSPTRDAECE |
| | | CVSGFHCLGAGCTMCQQDCKQGQELTKEGCKDCCLGTFNDQK |
| | | NGICRPWTNCSLEGKSVLANGTKKRDVVCGPPAADSFPDTSSVT |
| | | VPAPERKPDHHPQIITFFLALISAALLFLVFFLVVRFSVAKWGRKK |
| | | LLYIFKQPFIKPVQTAQEEDGCSCRFPEEEEGDCEL |
| 40 | Human CD137 | CPPNSFSSAGGQRTCDICRQCKGVFRTRKECSSTSNAECDC |
| | amino acids 48-88 | |
| 41 | Human CD137 | LQDPCSNCPAGTFCDNNRNQICSPCPPNSFSSAGGQRTCDICR |
| | (mature protein) | QCKGVFRTRKECSSTSNAECDCTPGFHCLGAGCSMCEQDCKQG |
| | | QELTKKGCKDCCFGTFNDQKRGICRPWTNCSLDGKSVLVNGTK |
| | | ERDVVCGPSPADLSPGASSVTPPAPAREPGHSPQIISFFLALTSTA |
| | | LLFLLFFLTLRFSVVKRGRKKLLYIFKQPFMRPVQTTQEEDGCSCR |
| | | FPEEEEGGCEL |
| 42 | VH-CD137-MOR7480- | EVQLVQSGAEVKKPGESLRISCKGSGYSFSTYWISWVRQMPGK |
| | FEAR | GLEWMGKIYPGDSYTNYSPSFQGQVTISADKSISTAYLQWSSLK |
| | | ASDTAMYYCARGYGIFDYWGQGTLVTVSS |
| 43 | VH-CD137-MOR7480- | GYSFSTYW |
| | FEAR CDR1 | |
| 44 | VH-CD137-MOR7480- | IYPGDSYT |
| | FEAR CDR2 | |
| 45 | VH-CD137-M0R7480- | ARGYGIFDY |
| | FEAR CDR3 | |
| 46 | VL-CD137-MOR7480 | SYELTQPPSVSVSPGQTASITCSGDNIGDQYAHWYQQKPGQSP |
| | (Lambda) | VLVIYQDKNRPSGIPERFSGSNSGNTATLTISGTQAMDEADYYC |
| | | ATYTGFGSLAVFGGGTKLTVL |
| 47 | VL-CD137- | NIGDQY |
| | MOR7480_CDR1 | |
| | VL-CD137- | QDK |
| | MOR7480_CDR2 | |
| 48 | VL-CD137- | ATYTGFGSLAV |
| | MOR7480_CDR3 | |
| 49 | Recombinant human | MYRMQLLSCIALSLALVTNSAPTSSSTKKTQLQLEHLLLDLQMIL |
| | interleukin analog | NGINNYKNPKLTRMLTFKFYMPKKATELKHLQCLEEELKPLEEVLN |
| | (Proleukin ® | LAQSKNFHLRPRDLISNINVIVLELKGSETTFMSEYADETATIVEF |
| | (aldesleukin)) | LNRWITFCQSIISTLT |
| 50 | MPDL3280A VH | EVQLVESGGGLVQPGGSLRLSCAASGFTFSDSWIHWYRQAPGK |
| | | GLEWYAWISPYGGSTYYADSVKGRFTISADTSKNTAYLQMNSLR |
| | | AEDTAVYYCARRHWPGGFDYWGQGTLVTVSS |
| 51 | MPDL3280A VL | DIQMTQSPSSLSASVGDRVTITCRASQDVSTAVAWYQQKPGKA |
| | | PKLLIYSASFLYSGVPSRFSGSGSGTDFTLTISSLQPEDFATYYCQ |
| | | QYLYHPATFGQGTKVEIK |

### EXAMPLES

### Example 1: Generation of CD137 Antibody

Antibodies CD137-009 was generated as described in example 1 of WO2016/110584. In short, rabbits were immunized with a mixture of proteins containing a human CD137-Fc fusion protein. Single B cells from blood were sorted and screened for production of CD137 specific antibody by ELISA and flow cytometry. From screening-positive B cells, RNA was extracted and sequencing was performed. The variable regions of heavy and light chain were gene synthesized and cloned into a human IgG1 kappa expression vector or human IgG1 lambda expression vector including a human IgG1 heavy chain containing the following amino acid mutations: L234F, L235E, D265A and F405L (FEAL) or K409R (FEAR) wherein the amino acid position number is according to EU numbering (correspond to SEQ ID NO: 25). The variable region sequences of the chimeric CD137 antibody (CD137-009) are shown in the Sequence Listing SEQ ID NO: 8 and SEQ ID NO: 12 herein.

### Example 2: Humanization of the Rabbit (Chimeric) CD137 Antibody

Humanized antibody sequences from the rabbit anti-CD137-009 were generated at Antitope (Cambridge, UK). Humanized antibody sequences were generated using germline humanization (CDR-grafting) technology. Humanized V region genes were designed based on human germline sequences with closest homology to the VH and Vκ amino acid sequences of the rabbit antibody. A series of seven VH and three Vκ (VL) germline humanized V-region genes were designed. Structural models of the non-human parental antibody V regions were produced using Swiss PDB and analyzed in order to identify amino acids in the V region frameworks that may be important for the binding properties of the antibody. These amino acids were noted for incorporation into one or more variant CDR-grafted antibodies. The germline sequences used as the basis for the humanized designs are shown in Table 2.

TABLE 2
Closest matching human germline V segment and J segment sequences.

| | Heavy chain | | Light chain (κ) | |
|---|---|---|---|---|
| | Human V region | Human J region | Human V region | Human J region |
| | germline | germline | germline | germline |
| Antibody | segment | segment | segment | segment |
| Rabbit anti-CD137-009 | hIGHV3-49*04 | hIGHJ4 | hIGKV1-33*01 | IGKJ4 |

Variant sequences with the lowest incidence of potential T cell epitopes were then selected using Antitope's proprietary in silico technologies, iTope™ and TCED™ (T Cell Epitope Database) (Perry, L. C. A, Jones, T. D. and Baker, M. P. New Approaches to Prediction of Immune Responses to Therapeutic Proteins during Preclinical Development (2008). Drugs in R&D 9 (6): 385-396; 20 Bryson, C. J., Jones, T. D. and Baker, M. P. Prediction of Immunogenicity of Therapeutic Proteins (2010). Biodrugs 24 (1): 1-8). Finally, the nucleotide sequences of the designed variants have been codon-optimized.

The variable region sequences of the humanized CD137 antibody (CD137-009-HC7LC2) are shown in the Sequence Listing SEQ ID NO: 15 and SEQ ID NO: 16 herein.

### Example 3: DNA Shuffling Between Wild Boar CD137 or Elephant CD137 and Human CD137 to Determine Domains Important for Binding of CD137 Antibody

To determine domains important for binding of the CD137 antibody to human CD137, DNA shuffling was performed between human and wild boar CD137 (Sus scrofa; XP_005665023) or between human and African elephant CD137 (Ioxodonta africana; XP_003413533). Shuffle constructs were prepared from DNA encoding human CD137, by replacing human domains with wild boar (shuffle construct 1-4, 6) or elephant (shuffle construct 5) domains. The amino acid sequence of the shuffle constructs are show in table 1.

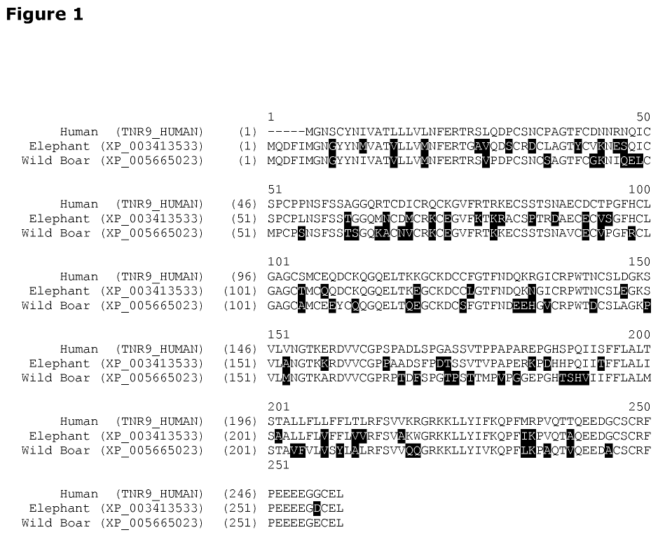

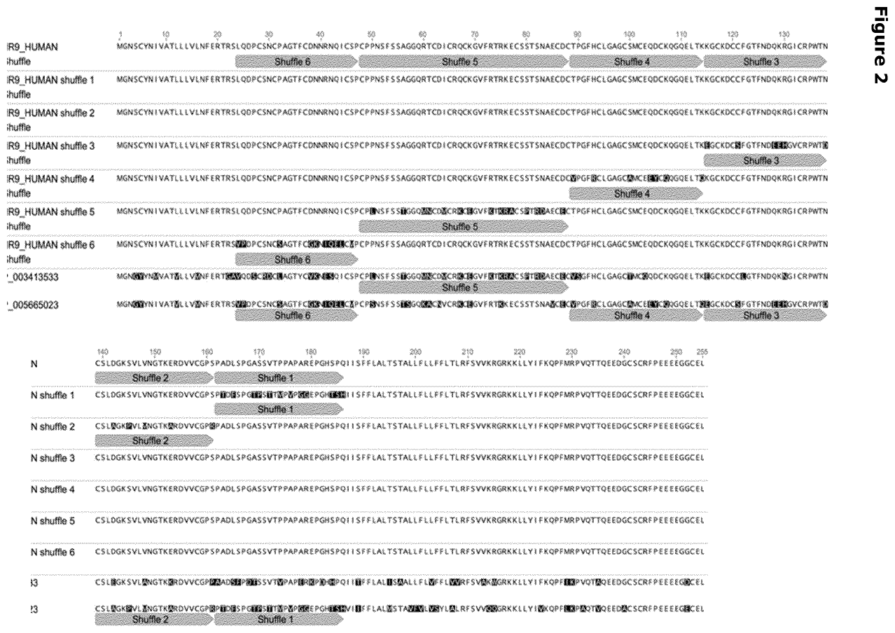

If a domain in human CD137 is important for binding of an anti-CD137 antibody, binding will be lost upon replacement of that domain with the wild boar or African elephant domain. Homology between human and wild boar and between human and African elephant CD137 is 70.2 and 74.5%, respectively. Requirement for the selection of these two species was that the domain of interest in African elephant and wild boar was sufficiently different compared to human, resulting in loss of binding, while remaining critical structural interactions which is necessary to minimize the risk of misfolding or loss of expression. FIG. 1 shows sequence alignments of human, wild boar and African elephant CD137. FIG. 2 shows the constructs for human CD137 containing wild boar CD137 or African elephant CD137 domains, as indicated.

3×10⁶ HEK293T-17 cells were seeded in T75 culture flasks (Greiner Bio-One, cat. no. 658175) in 20 mL RPMI 1640 GlutaMAX medium containing 10% FCS (Biochrom, cat. no. S 0115). After O/N incubation, cells were transiently transduced with expression vectors encoding the shuffle constructs or the wild boar, African elephant or human CD137 downstream of a constitutively active human elongation factor-1 alpha (EF-1 alpha) promotor using TransIT®-LT1 Transfection Reagent, Mirus Bio (VWR International, cat. no. 731-0029), according to the manufacturer's instructions. The next day, cells were harvested using 1.5 mL Accutase (Sigma Aldrich, cat. no. A6964) (incubation at 37° C. for 5 min.) and flow cytometry was performed, essentially as described supra, to measure surface expression of the shuffle constructs and the human, African elephant and wild boar CD137 and to measure binding of the antibody clones to the different shuffle constructs. To measure cell surface expression of the constructs, transduced cells were incubated with 1 μg/mL goat polyclonal anti-human CD137 (R&D Systems, cat. no. AF838) in FACS buffer (4° C., 20 min.), followed by incubation with APC-labeled anti-goat IgG (H+L) (R&D Systems, cat. no. F0108) (4° C., 20 min.). Binding of the different CD137 antibody clones to cells expressing the shuffle constructs was measured by incubation of the transduced cells with 1 μg/mL of the antibody clones, followed by APC-labeled AffiniPure F(ab′)2 Fragment (1:50 final dilution; Jackson, cat. no. 109-136-127).

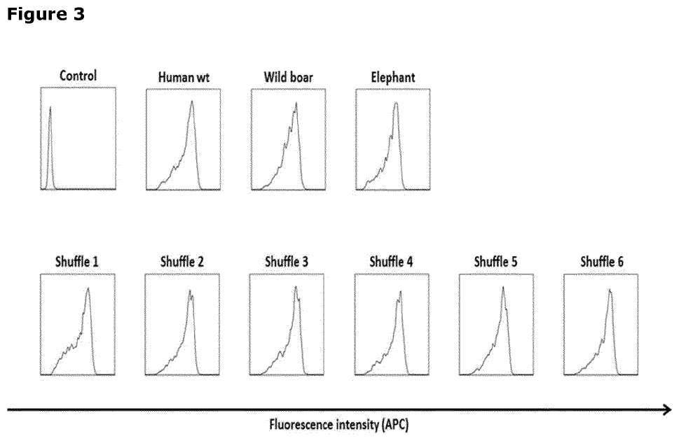

All CD137 shuffle constructs, as well as human, African elephant and wild boar CD137, were expressed on the cell surface with similar expression levels (FIG. 3).

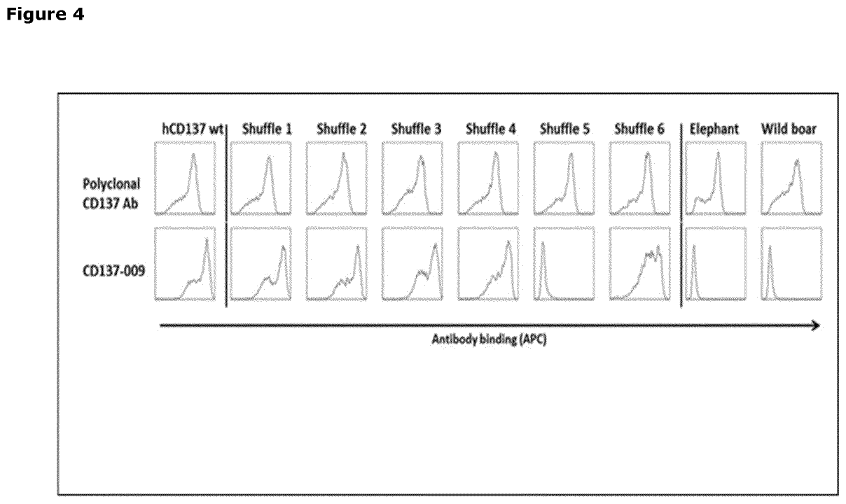

FIG. 4 shows that CD137-009 showed loss of binding to African elephant and wild boar CD137. CD137-009 also showed loss of binding to shuffle construct 5, as compared to binding to human CD137.

### Example 4: Generation of PD-L1 Antibody

Immunization and hybridoma generation were performed at Aldevron GmbH (Freiburg, Germany). A cDNA encoding amino acid 19-238 of human PD-L1 was cloned into Aldevron proprietary expression plasmids. Antibody PD-L1-547 was generated by immunization of OmniRat animals (transgenic rats expressing a diversified repertoire of antibodies with fully human idiotypes; Ligand Pharmaceuticals Inc., San Diego, USA) using intradermal application of human PD-L1 cDNA-coated gold-particles using a hand-held device for particle-bombardment (“gene gun”). Serum samples were collected after a series of immunizations and tested in flow cytometry on HEK cells transiently transfected with the aforementioned expression plasmids to express human PD-L1. Antibody-producing cells were isolated and fused with mouse myeloma cells (Ag8) according to standard procedures. RNA from hybridomas producing PD-L1 specific antibody was extracted and sequencing was performed. The variable regions of heavy and light chain (SEQ ID NO:17 and 21) were gene synthesized and cloned into a human IgG1 lambda expression vector including a human IgG1 heavy chain containing the following amino acid mutations: L234F, L235E, D265A and K409R (FEAR) wherein the amino acid position number is according to EU numbering (correspond to SEQ ID NO:24).

### Example 5: Generation of Bispecific Antibodies by 2-MEA-Induced Fab-Arm Exchange

Bispecific IgG1 antibodies were generated by Fab-arm-exchange under controlled reducing conditions. The basis for this method is the use of complementary CH3 domains, which promote the formation of heterodimers under specific assay conditions as described in WO2011/131746. The F405L and K409R (EU numbering) mutations were introduced into the relevant antibodies to create antibody pairs with complementary CH3 domains.

To generate bispecific antibodies, the two parental complementary antibodies, each antibody at a final concentration of 0.5 mg/mL, were incubated with 75 mM 2-mercaptoethylamine-HCl (2-MEA) in a total volume of 100 μL PBS at 31° C. for 5 hours. The reduction reaction was stopped by removing the reducing agent 2-MEA using spin columns (Microcon centrifugal filters, 30 k, Millipore) according to the manufacturer's protocol.

Bispecific antibodies were generated by combining the following antibodies from Example 1 and 4:
CD137-009-FEAL antibody combined with the PD-L1-547-FEAR antibody
PD-L1-547-FEAL antibody combined with the CD137-009-FEAR
PD-L1-547-FEAL antibody combined with CD137-009-HC7LC2-FEAR antibody,
b12-FEAL antibody combined with the PD-L1-547-FEAR antibody, with CD137-009-FEAR or with CD137-009-HC7LC2-FEAR antibody, using as the first arm the antibody b12 which is a gp120 specific antibody (Barbas, CF. J Mol Biol. 1993 Apr. 5; 230(3):812-23)
PD-L1-547-FEAL or CD137-009-FEAL with b12-FEAR antibody.

### Example 6: Effect of PD-L1 Antibody on the PD-1/PD-L1 Interaction

The effect of monovalent PD-L1 antibody b12-FEALxPD-L1-547-FEAR on the interaction of PD-1 and PD-L1 was determined in a PD-1/PD-L1 inhibition bioassay as developed by Promega (Madison, USA). This is a bioluminescent cell-based assay consisting of two genetically engineered cell lines: PD-1 effector cells, which are Jurkat T cells expressing human PD-1 and a luciferase reporter driven by an NFAT response element (NFAT-RE), and PD-L1 aAPC/CHO-K1 cells, which are CHO-K1 cells expressing human PD-L1 and an engineered cell surface protein designed to activate cognate TCRs in an antigen-independent manner. When the two cell types are co-cultured, the PD-1/PD-L1 interaction inhibits TCR signaling and NFAT-RE-mediated luminescence. Addition of an antibody that blocks the PD-1/PD-L1 interaction releases the inhibitory signal and results in TCR activation and NFAT-RE-mediated luminescence.

PD-L1 aAPC/CHO-K1 cells (Promega, cat. no. J109A) were thawed according to the manufacturer's protocol, resuspended Ham's F12 medium (Promega, cat. no. J123A) containing 10% Fetal Bovine Serum (FBS; Promega, cat. no. J121A), and plated in 96 well flat bottom culture plates (CulturPlate-96, Perkin Elmer, cat. no. 6005680). Plates were incubated for 16 hours at 37° C., 5% CO₂. Supernatant was removed and serial dilutions of antibody (final concentration ranging from 5 to 0.001 μg/mL; 4-fold dilutions in RPMI 1640 [Lonza, cat. no. BE12-115F] containing 1% Fetal Bovine Serum [FBS; Promega, cat. no. J121A]) were added. PD-1 effector cells (Promega, cat. no. J115A; thawed according to the manufacturer's protocol and resuspended in RPMI/1% FBS) were added. Plates were incubated for 6 h at 37° C., 5% CO₂. After equilibration to room temperature, 40 μl Bio-Glo reagent (Bio-Glo luciferase assay substrate [Promega cat. no. G72013] reconstituted in Bio-Glo luciferase assay buffer [Promega, cat. no. G7198] according to the manufacturer's protocol) was added to each well. Plates were incubated at room temperature for 5-10 minutes and luminescence was measured using an EnVision Multilabel Reader (PerkinElmer). The effect on PD1-PD-L1 interaction, relative to control (without antibody added), was calculated as follows:

Fold induction=RLU (induced-background)/RLU (no antibody control-background), RLU is relative light units

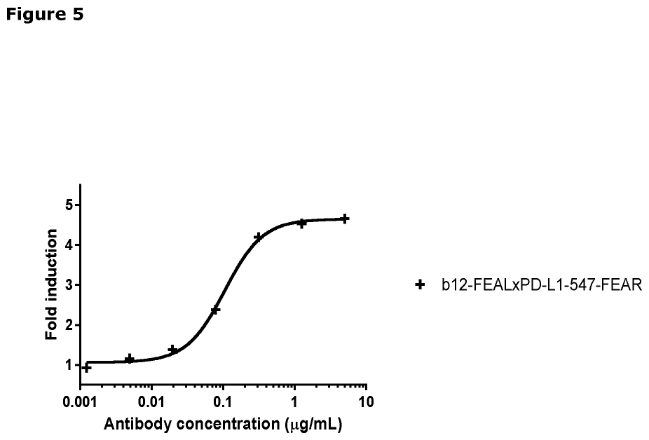

FIG. 5 shows that monovalent antibody b12-FEALxPD-L1-547-FEAR efficiently inhibited PD1-PD-L1 interaction.

### Example 7: Antigen-Specific CD⁸⁺ T Cell Proliferation Assay to Measure Effects by Bispecific Antibodies Binding to PD-L1 and CD137

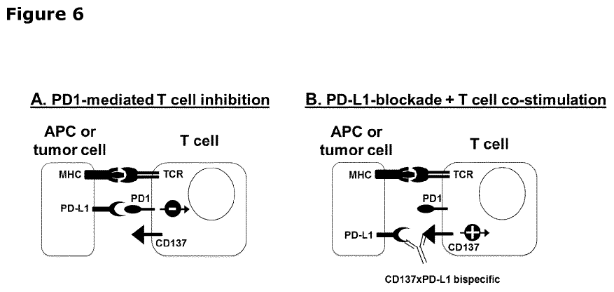

A schematic representation of the anticipated mode of action of CD137xPD-L1 bispecific antibodies is shown in FIG. 6.

To measure induction of T cell proliferation by the bispecific antibody targeting PD-L1 and CD137 in an antigen-specific assay, dendritic cells (DCs) were transfected with claudin-6 in vitro-transcribed RNA (IVT-RNA) to express the claudin-6 antigen. T cells were transfected with PD-1 IVT-RNA and with the claudin-6-specific, HLA-A2-restricted T cell receptor (TCR). This TCR can recognize the claudin-6-derived epitope presented in HLA-A2 on the DC. The CD137xPD-L1 bispecific antibody can cross-link PD-L1 endogenously expressed on monocyte-derived dendritic cells or on tumor cells and CD137 on the T cells, leading to inhibition of the inhibitory PD-1/PD-L1 interaction and at the same time clustering of CD137, resulting in T cell proliferation. Clustering of the CD137 receptor expressed on T cells leads to activation of the CD137 receptor which thereby delivers a co-stimulatory signal to the T cell.

HLA-A2⁺ peripheral blood mononuclear cells (PBMCs) were obtained from healthy donors (Transfusionszentrale, University Hospital, Mainz, Germany). Monocytes were isolated from PBMCs by magnetic-activated cell sorting (MACS) technology using anti-CD14 MicroBeads (Miltenyi; cat. no. 130-050-201), according to the manufacturer's instructions. The peripheral blood lymphocytes (PBLs, CD14-negative fraction) were frozen for future T-cell isolation. For differentiation into immature DCs (iDCs), 1×10⁶ monocytes/ml were cultured for five days in RPMI GlutaMAX (Life technologies GmbH, cat. no. 61870-044) containing 5% human AB serum (Sigma-Aldrich Chemie GmbH, cat. no. H4522-100ML), sodium pyruvate (Life technologies GmbH, cat. no. 11360-039), non-essential amino acids (Life technologies GmbH, cat. no. 11140-035), 100 IU/mL penicillin-streptomycin (Life technologies GmbH, cat. no. 15140-122), 1000 IU/mL granulocyte-macrophage colony-stimulating factor (GM-CSF; Miltenyi, cat. no. 130-093-868) and 1,000 IU/mL interleukin-4 (IL-4; Miltenyi, cat. no. 130-093-924). Once during these five days, half of the medium was replaced with fresh medium. iDCs were harvested by collecting non-adherent cells and adherent cells were detached by incubation with PBS containing 2 mM EDTA for 10 min at 37°. After washing, iDCs were frozen in RPMI GlutaMAX containing 10% v/v DMSO (AppliChem GmbH, cat. no A3672,0050)+50% v/v human AB serum for future antigen-specific T cell assays.

One day prior to the start of an antigen-specific CD⁸⁺ T cell proliferation assay, frozen PBLs and iDCs, from the same donor, were thawed. CD8⁺ T cells were isolated from PBLs by MACS technology using anti-CD8 MicroBeads (Miltenyi, cat. no. 130-045-201), according to the manufacturer's instructions. About 10-15×10⁶ CD⁸⁺ T cells were electroporated with 10 μg of in vitro translated (IVT)-RNA encoding the alpha-chain plus 10 μg of IVT-RNA encoding the beta-chain of a claudin-6-specific murine TCR (HLA-A2-restricted; described in WO 2015150327 A1) plus 10 μg IVT-RNA encoding PD-1 in 250 μL X-Vivol5 (Biozym Scientific GmbH, cat. no. 881026) in a 4-mm electroporation cuvette (VWR International GmbH, cat. no. 732-0023) using the BTX ECM® 830 Electroporation System device (BTX; 500 V, 1×3 ms pulse). Immediately after electroporation, cells were transferred into fresh IMDM medium (Life Technologies GmbH, cat. no. 12440-061) supplemented with 5% human AB serum and rested at 37° C., 5% CO₂ for at least 1 hour. T cells were labeled using 1.6 μM carboxyfluorescein succinimidyl ester (CFSE; Invitrogen, cat. no. C34564) in PBS according to the manufacturer's instructions, and incubated in IMDM medium supplemented with 5% human AB serum, O/N.

Up to 5×10⁶ thawed iDCs were electroporated with 5 μg IVT-RNA encoding full length claudin-6, in 250 μL X-Vivol5 medium, using the electroporation system as described above (300 V, 1×12 ms pulse) and incubated in IMDM medium supplemented with 5% human AB serum, O/N.

The next day, cells were harvested. Cell surface expression of claudin-6 and PD-L1 on DCs and TCR and PD-1 on T cells was checked by flow cytometry. DCs were stained with an Alexa647-conjugated CLDN6-specific antibody (non-commercially available; in-house production) and with anti-human CD274 antibody (PD-L1, eBioscienes, cat. no. 12-5983) and T cells were stained with an anti-Mouse TCR β Chain antibody (Becton Dickinson GmbH, cat. no. 553174) and with anti-human CD279 antibody (PD-1, eBioscienes, cat. no. 17-2799). 5,000 electroporated DCs were incubated with 50,000 electroporated, CFSE-labeled T cells in the presence of bispecific or control antibodies in IMDM GlutaMAX supplemented with 5% human AB serum in a 96-well round-bottom plate. T cell proliferation was measured after 5 days by flow cytometry. Detailed analyses of T cell proliferation based on CFSE-peaks indicating cell divisions were made by FlowJo software. In the results, ‘% divided cells’ indicates percentage of cells that went into division and ‘proliferation index’ indicates average number of divisions of cells that went into division

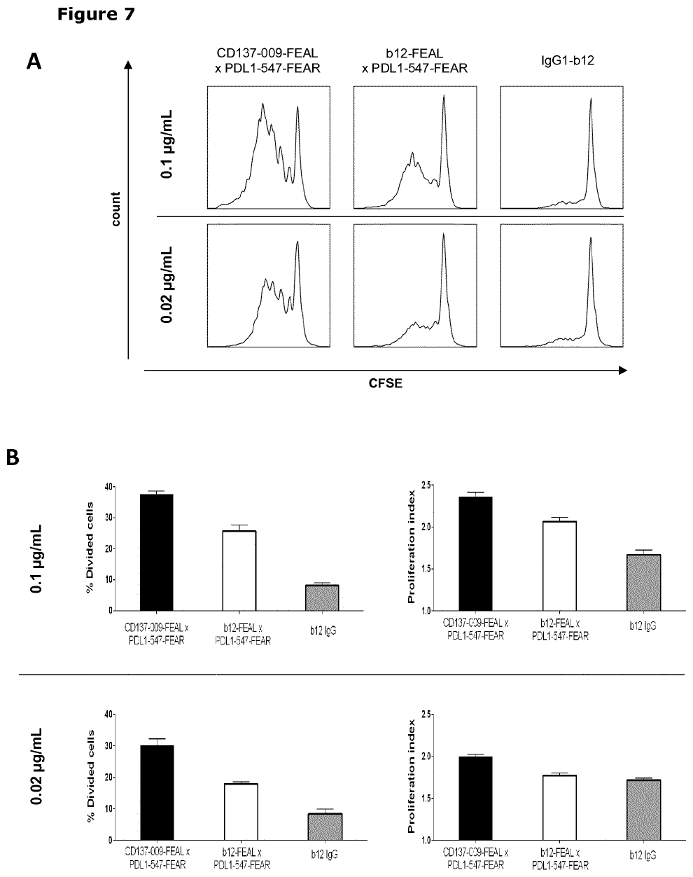

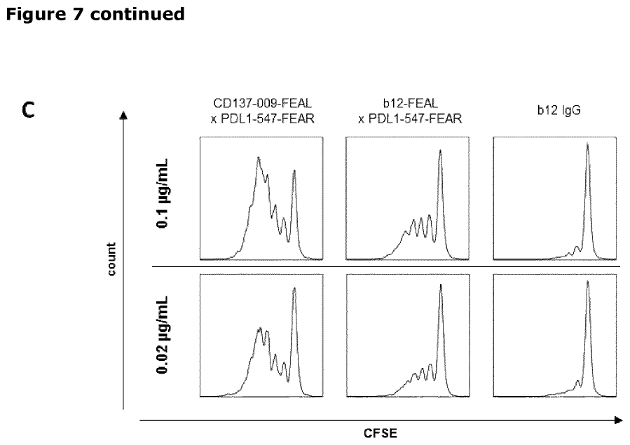

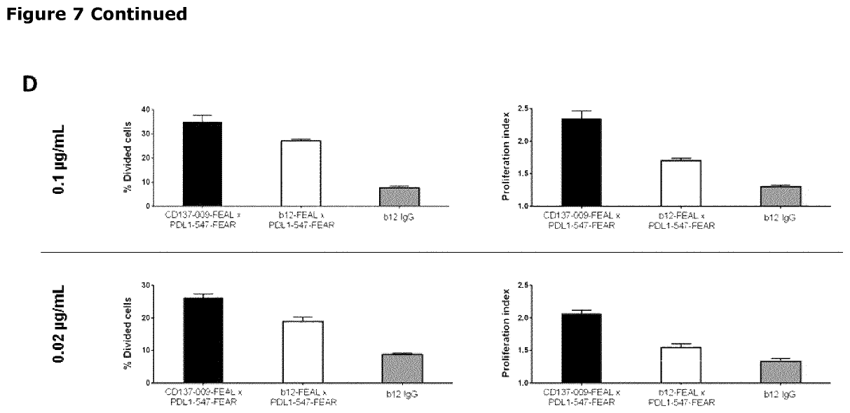

The monovalent PD-L1-control antibody having one irrelevant binding-arm, b12-FEALxPD-L1-547-FEAR, enhanced T cell proliferation to a certain extent compared to incubation with b12 (as regular IgG1), and the bispecific antibody CD137-009-FEALxPD-L1-547-FEAR induced strong proliferation of CD⁸⁺ T cells (FIG. 7). This was reflected by both an increase in the percentage of divided cells (FIGS. 7B and D left panels) as well as an increase of the proliferation index (FIGS. 7B and D right panels).

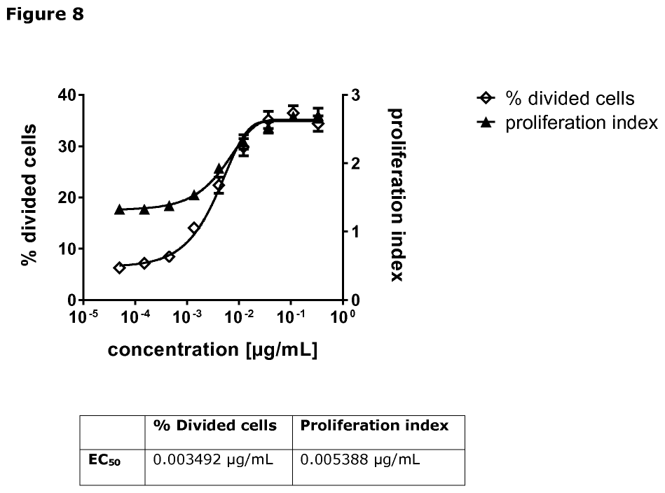

In addition, the EC₅₀ value in this assay was determined for CD137-009-FEALxPD-L1-547-FEAR. To this end, the bispecific antibody was analyzed at 3-fold serial dilutions from 1 to 0.00015 μg/mL (FIG. 8). Percentage of divided cells and proliferation index were determined by FlowJo software. Curves were analyzed by non-linear regression (sigmoidal dose-response with variable slope) using GraphPad Prism 5 software (GraphPad Software, San Diego, Calif., USA). The EC₅₀ values of the induction of antigen-specific T cell proliferation of CD137-009-FEALxPD-L1-547-FEAR was 0.003492 μg/mL for ‘% divided cells’ and 0.005388 μg/mL for ‘proliferation index’.

### Example 8: Comparison of the Bispecific Antibody Targeting PD-L1 and CD137 with a Combination of Two Monovalently Binding CD137 and PD-L1 Antibodies or the Two Parental Antibodies (PD-L1-547+CD137-009) in an Antigen-Specific T-Cell Assay with Active PD1/PD-L1 Axis

To measure induction of T cell proliferation by the bispecific antibody targeting PD-L1 and CD137, an antigen-specific T cell proliferation assay with active PD1/PD-L1 axis was performed (general assay set-up analogous to example 7). In short, 5,000 claudin-6-IVT-RNA electroporated DCs were incubated with 50,000 claudin-6-specific TCR- and PD1-IVT-RNA electroporated, CFSE-labeled T cells in the presence of bispecific or control antibodies in IMDM GlutaMAX supplemented with 5% human AB serum in a 96-well round-bottom plate. T cell proliferation was measured after 5 days by flow cytometry. Detailed analyses of T cell proliferation based on CFSE-peaks indicating cell divisions were performed using FlowJo software. In the results, ‘% divided cells’ indicates percentage of cells that went into division and ‘proliferation index’ indicates average number of divisions of cells that went into division.

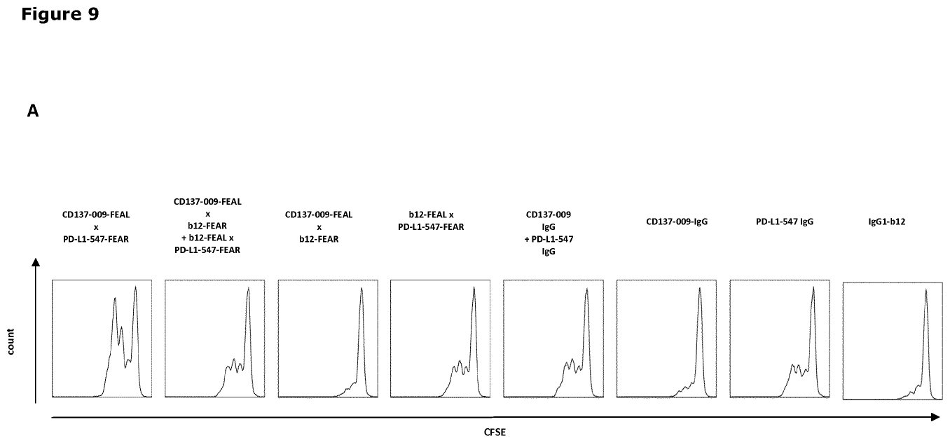

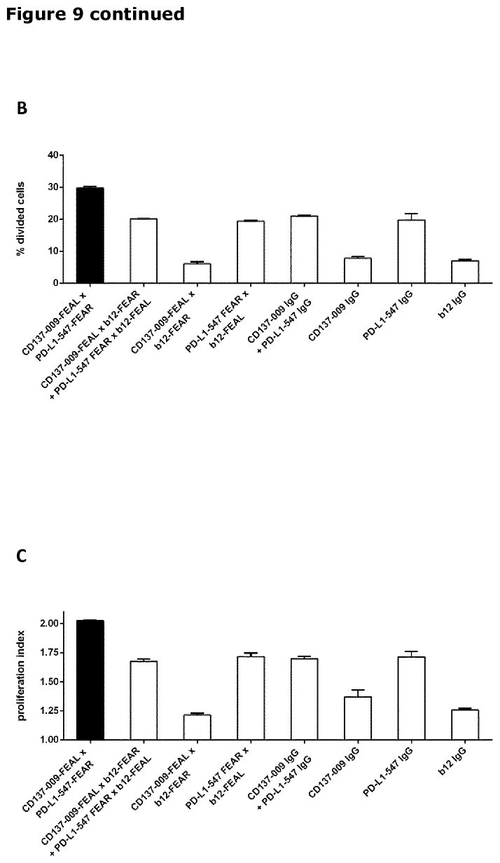

Neither the monovalent CD137-control antibody, CD137-009-FEALxb12-FEAR, having one irrelevant binding-arm nor the corresponding bivalent parental antibody CD137-009 had an effect on T cell proliferation when compared IgG1-b12. In contrast, incubation with the monovalent PD-L1-control antibody as well as the bivalent parental antibody (b12-FEALxPD-L1-547-FEAR and PD-L1-547, respectively) led to a moderately enhanced T-cell proliferation compared to incubation with IgG1-b12 control antibody. A comparable level of T-cell proliferation was detectable upon incubation with the combined monovalent control antibodies (CD137-009-FEALxb12-FEAR+b12-FEALxPD-L1-547-FEAR) and the combined corresponding parental antibodies (CD137-009+PD-L1-547). In contrast, the bispecific antibody CD137-009-FEALxPD-L1-547-FEAR induced strong proliferation of CD⁸⁺ T cells, which was superior to both combined controls (monovalent and bivalent) (FIG. 9). This was reflected by both an increase in the percentage of divided cells (FIG. 9B) as well as an increase in the proliferation index (FIG. 9C).

### Example 9: Ex Vivo TIL Expansion Assay to Evaluate the Effects of the CD137xPD-L1 Bispecific Antibody on Tumor Infiltrating Lymphocytes

To evaluate the effects of CD137-009-FEALxPD-L1-547-FEAR on tumor infiltrating lymphocytes (TIL), an ex vivo culture of human tumor tissue was performed as follows. Fresh human tumor tissue resection specimens were washed three times by transferring the isolated tumor chunks from one well in a 6-well plate (Fisher Scientific cat. no. 10110151) containing wash medium to the next using a spatula or serological pipette. Wash medium was composed of X-VIVO 15 (Biozym, cat. no. 881024) supplemented with 1% Pen/Strep (Thermo Fisher, cat. no. 15140-122) and 1% Fungizone (Thermo Fisher, cat. no. 15290-026). Next, the tumor was dissected with a surgical knife (Braun/Roth, cat. no. 5518091 BA223) and cut into pieces with a diameter of about 1-2 mm. Two pieces each were put into one well of a 24-well plate (VWR international, cat. no. 701605) containing 1 mL TIL medium (X-VIVO 15, 10% Human Serum Albumin (HSA, CSL Behring, cat. no. PZN-6446518) 1% Pen/Strep, 1% Fungizone and supplemented with 10 U/mL IL-2 (Proleukin®S, Novartis Pharma, cat. no. 02238131)). CD137-009-FEALxPD-L1-547-FEAR was added at the indicated final concentrations. Culture plates were incubated at 37° C. and 5% CO₂. After 72 hours, 1 mL of fresh TIL medium containing the indicated concentration of the bispecific antibody was added to each well. Wells were monitored via a microscope for the occurrence of TIL clusters every other day. Wells were transferred individually when more than 25 TIL microclusters were detected in the respective well. To split TIL cultures, the cells in the wells of a 24-well plate were re-suspended in the 2 mL medium and transferred into a well of a 6-well plate. Each well was in addition supplemented with another 2 mL of TIL medium.

After a total culture period of 10-14 days, TILs were harvested and analyzed by flow cytometry. Cells were stained with the following reagents, all diluted 1:50 in staining-buffer, (D-PBS containing 5% FCS and 5 mM EDTA), anti-human CD4-FITC (Miltenyi Biotec, cat. no. 130-080-501), anti-human CD3-PE-Cy7 (BD Pharmingen, cat. no. 563423), 7-aminoactinomycin D (7-AAD, Beckman Coulter, cat. no. A07704), anti-human CD56-APC (eBioscience, cat. no. 17-0567-42), and anti-human CD8-PE (TONBO, cat. 50-0088). To allow for quantitative comparison of the acquired cells between different treatment groups, cell pellets were re-suspended after the last washing step in FACS-buffer supplemented with BD™ CompBeads (BD biosciences, cat. no. 51-90-9001291). Flow cytometric analysis was performed on a BD FACSCanto™ II flow cytometer (Becton Dickinson) and acquired data was analyzed using FlowJo 7.6.5 software. The relative viable TIL count, CD3+CD⁸⁺ T cell count, CD3⁺ CD4⁺ T cell count and CD3⁻CD56⁺ NK cell count per 1,000 beads correlating to the corresponding well in a 6-well plate was calculated by normalization of the acquired 7AAD-negative cell fraction to the acquired bead counts.

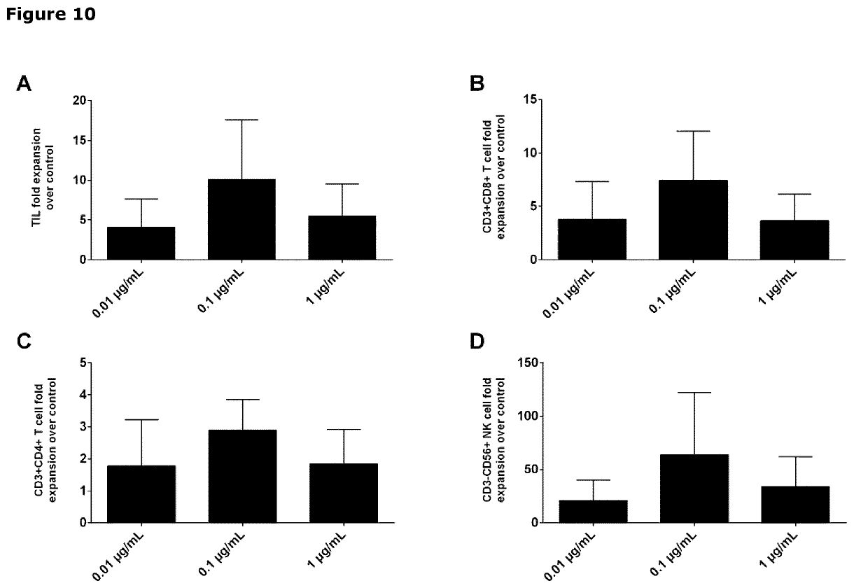

FIG. 10 shows the analysis of a TIL expansion from a human non-small-cell lung carcinoma tissue specimen. Here, the following concentrations of CD137-009-FEALxPD-L1-547-FEAR were added: 0.01, 0.1 and 1 μg/mL; a tissue specimen from the same patient without antibody addition served as negative control. After 10 days of culture, the TILs were harvested and analyzed by flow cytometry. Five samples (from 5 original wells) for each antibody concentration derived from different wells of the 24-well plate were measured. In all samples cultured with the bispecific antibody the viable count of TILs was significantly increased in comparison to the without antibody control samples. Overall, an up to 10-fold expansion of viable TILs was observed, when 0.1 μg/mL CD137-009-FEALxPD-L1-547-FEAR was added to cultures (FIG. 10 A). CD3+CD⁴⁺ T helper cells were only slightly expanded (FIG. 10 C; 2.8-fold expansion), whereas in contrast, the most prominent TIL expansion was seen for CD3⁻CD56⁺ NK cells (FIG. 10 D; up to 64-fold expansion over control). Also a strong effect on CD3⁺ CD8⁺ cytotoxic T lymphocytes (CTLs) was observed (FIG. 10 B; 7.4-fold expansion over control).

### Example 10: Effect of a Surrogate Bispecific Mouse Antibody Binding to mPD-L1 and mCD137 on Ovalbumin Specific T Cell Proliferation in C57BL/6 Mice after OT-I CD8⁺ Adoptive T Cell Transfer

Surrogate mouse bispecific antibodies mCD137-3H3xmPD-L1-MPDL3280A, mCD137-3H3xb12 and mPD-L1-MPDL3280Axb12 were generated using a method to generate murine bispecific antibodies based on controlled Fab-arm exchange (Labrijn et al, 2017 Sci Rep. 7(1): 2476 and WO2016097300).

The monoclonal antibody 3H3, which binds to mouse 4-1BB, was obtained from BioXcell (cat. No. BE0239) and protein sequenced at ProtTech. The inferred cDNA sequence was deducted using proprietary methods. The variable regions of heavy and light chain were gene synthesized and cloned into a mouse IgG2a expression vector including a murine IgG2a constant region containing the following amino acid mutations: L234A, L235A, F405L and R411T. Similarly, the variable regions of b12 were cloned into this expression vector.

The antibody MPDL3280A (heavy and light chain variable sequences set forth in SEQ ID NOs: 50 and 51, respectively) has been described to bind both human and mouse PD-L1. The variable regions of heavy and light chains of this antibody were cloned into a mouse IgG2a expression vector including a murine IgG2a constant region containing the following amino acid mutations: L234A, L235A, T370K and K409R.

Bispecific mouse (in essence rat-human-mouse chimeric) antibodies were generated by Fab-arm-exchange under controlled reducing conditions as described supra.

Female C57BL/6JO1aHsd mice (Envigo RMS GmbH, Rossdorf, Germany), 6-8 weeks of age, with a weight between 17 and 24 g, were acclimated to the animal facility for at least six days prior to study enrollment. These mice were used as recipients. Female or male C57BL/6 Thy1.1×C57BL/6J OT-1 mice homozygous for both the OT-1 and Thy1.1 allele were bred in-house (cross-bred from C57BL/6-Tg(TcraTcrb)1100Mjb/Crl and B6. PL-Thyla/CyJ mice) and were used as donors. Mice had free access to food (ssniff M-Z autoclavable Soest, Germany) and sterile water and were housed on 12 hours light/dark cycle at 22° C.±2° C. with a relative humidity of 55%±15%.

At the day of study start, C57BL/6 Thy1.1×C57BL/6J OT-1 donor mice were sacrificed and spleens were isolated. Spleens were mechanically dissociated and erythrocytes were lysed by re-suspending the splenocyte pellet with erythrocyte-lysis buffer (8.25 g/L NH₄Cl, 1 g/L KHCO3, 0.1 mM EDTA, pH7). Subsequently, splenocytes were washed with Dulbecco's PBS (DPBS) and CD⁸⁺ T cells were isolated using the CD8a (Ly-2) MicroBeads, mouse in combination with the autoMACS Pro Separator (both Miltenyi Biotec GmbH, Bergisch Gladbach, Germany). CD8⁺/OT-1⁺/Thy1.1⁺ T cells (2.5-5×10⁵ cells) were injected retro-orbitally in a total volume of 200 μL per C57BL/6JO1aHsd recipient mouse. The day after adoptive cell transfer, recipient mice were ‘vaccinated’ retro-orbitally with 100 μg ovalbumin/200 μL PBS as antigenic stimulus. After 6 hours, the mice were treated retro-orbitally with the respective bispecific antibody. In detail, 100 μg or 20 μg mCD137-3H3xmPD-L1-MPDL3280A, mCD137-3H3xb12 or mPD-L1-MPDL3280Axb12 antibody was injected per mouse. Injection of plain PBS was used as baseline reference and untreated animals (mice that received donor cells only) were used as negative control. After 6 days, 100 μL blood was drawn via the retro-orbital route and analyzed for Thy1.1+CD⁸⁺ T cells on a BD FACSCanto II cytometer (Becton Dickinson GmbH) using V500 rat anti-mouse CD45 (Becton Dickinson GmbH, Cat No. 561487), FITC rat anti-mouse CD8a (Life technologies, Cat No. MCD0801) and Alexa Fluor 647 anti-rat CD90/mouse CD90.1 (BioLegend Europe, Cat No. 202508) antibodies. Thy1.1 (CD90.1) positivity was used as surrogate for OT-1 specific T cells.

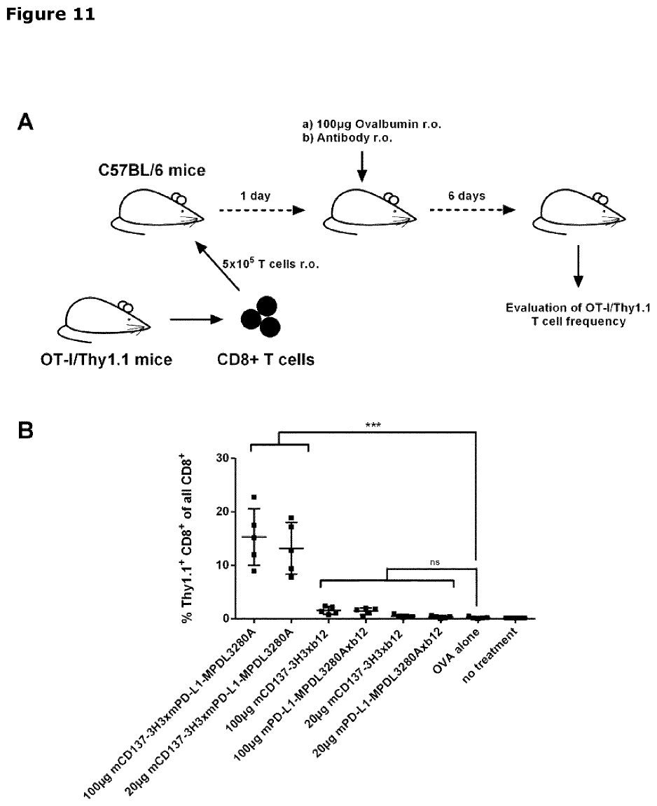

FIG. 11 A is a schematic representation of the OT-1 adoptive T-cell transfer assay outline. FIG. 11 B shows the analysis of the Thy1.1⁺CD⁸⁺ T-cell frequencies as determined by flow cytometry. For each bispecific antibody treatment modality, n=5 mice were used. The ovalbumin antigenic stimulus alone led to detectable increase in Thy1.1+CD⁸⁺ T-cell frequency compared to untreated animals. Interestingly, both monovalent control antibodies having one irrelevant b12 binding-arm, mCD137-3H3-xb12 and mPD-L1-MPDL3280Axb12, were not able to boost ovalbumin-specific OT-1 T-cell expansion compared to animals that had been treated with ovalbumin only. In contrast, the bispecific antibody mCD137-3H3xmPD-L1-MPDL3280A was able to induce a strong proliferation of OT-1 T cells leading to T-cell frequencies of 10-20% CD8+/OT-1+/Thy1.1⁺ T-cells (% of total T cell population) at both dose levels tested (20 and 100 μg antibody).

### Example 11: Effect of a Surrogate Bispecific Mouse Antibody Binding to mPD-L1 and mCD137 on Tumor Growth in a Subcutaneous, Syngeneic CT26 Mouse Tumor Model

Female BALB/c Rj mice (Janvier, Genest-St.-Isle, France), 6-8 weeks of age, with a weight between 17 and 24 g, were acclimated for at least six days prior to study enrollment. Mice had free access to food (ssniff M-Z autoclavable Soest, Germany) and sterile water and were housed on 12 hours light/dark cycle at 22° C.±2° C. with a relative humidity of 55%±10%. CT26 cells were obtained from the ATCC® (Cat No. CRL-2638™) and cultured in Roswell Park Memorial Institute medium (RPMI) 1640 Medium, GlutaMAX′ (Life technologies, Cat No. 61870-044) supplemented with 10% Fetal Bovine Serum (FBS) (Biochrom, Cat No. S 0115) in 5% CO₂ at 37° C. The cells were harvested using StemPro® Accutase® Cell Dissociation Reagent (Life technologies, Cat No. A1110501), resuspended in DPBS (Life technologies, Cat No. 14190-169), and 0.5×10⁶ cells/100 μl per mouse subcutaneously (SC) implanted into the right shaven flank of female BALB/c Rj mice. Tumor volume was assessed by caliper measurements every 2-3 days and is expressed as the product of the perpendicular diameters using the following formula: a²×b/2 where b is the longer of the two diameters (a<b). Animals were stratified into four groups when a mean tumor volume of 30 mm³ was reached. Treatment started the next day with intraperitoneal injection of 20 μg bispecific antibody binding to mPD-L1 and mCD137 (mCD137-3H3xmPD-L1-MPDL3280A), with the monovalent mCD137- or mPD-L1-control antibodies having one irrelevant binding-arm (mCD137-3H3xb12 and mPD-L1-MPDL3280Axb12), or PBS as negative control. Dosing schedule was every 2-3 days for the first eight injections, followed by an injection every 7 days until the end of the experiment. At day 29 post tumor cell inoculation, 100 μL blood was drawn via the retro-orbital route and analyzed for gp70-specific CD8⁺ T cells (gp70 is an envelope protein expressed on CT26 tumor cells) on a BD FACSCanto II cytometer (Becton Dickinson GmbH) using V500 rat anti-mouse CD45 (Becton Dickinson GmbH, Cat No. 561487), FITC rat anti-mouse CD8a (Life technologies, Cat No. MCD0801) antibodies and T-Select H-2Ld MuLV gp70 tetramer-SPSYVYHQF-APC (MBL Ltd. Corp., Cat No. TS-M521-2).

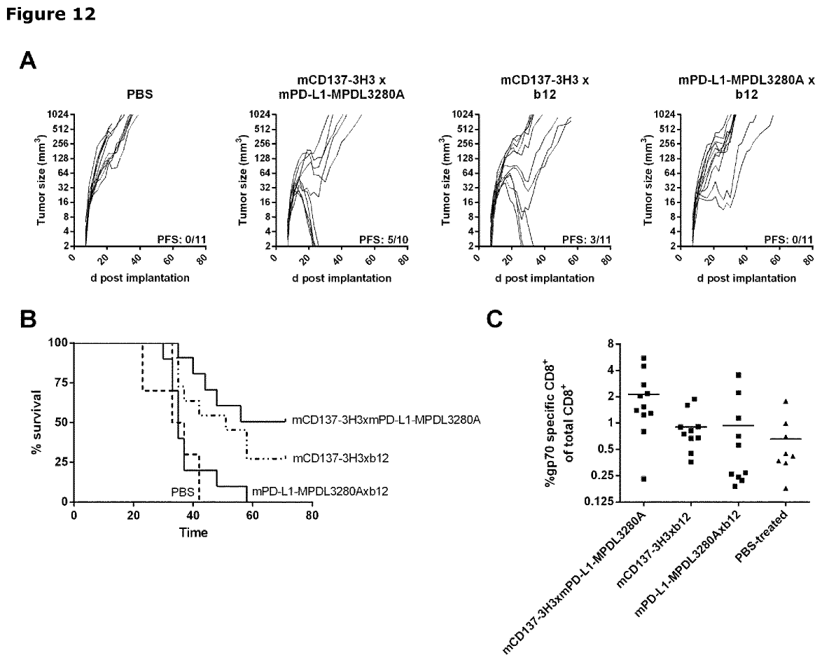

FIG. 12 A shows the tumor growth curves for all four treatment groups with individual lines being representative of a single tumor/mouse. Progression-free survival (PFS) frequencies for the respective treatment groups are given at the bottom of each plot. FIG. 12 B displays the corresponding Kaplan-Meier survival curves until the end of the experiment at day 71 post tumor cell inoculation. FIG. 12 C shows the analysis of gp70 tetramer₊ CD⁸⁺ T-cell frequencies as determined by flow cytometry. For each treatment modality, all mice that were still alive at day 29 post tumor cell implantation were analyzed. In summary, the bispecific antibody binding to mPD-L1 and mCD137 (mCD137-3H3xmPD-L1-MPDL3280A) provided most efficient tumor control with 5 out of 10 (i.e. 50%) animals going into complete tumor regression. In comparison, a slightly weaker but still prominent anti-tumor effect was observed for the mCD137-3H3xb12 control; treatment led to 3 out of 11 (i.e. 27%) animals being able to reject tumors. In both cases, all mice that went into full remission remained tumor-free until the end of the experiment. In striking contrast, both the mPD-L1-MPDL3280Axb12-treated cohort as well as the PBS control were not able to control tumor burden, with mPD-L1-MPDL3280Axb12-treatment leading at least to some intermittent tumor growth inhibition in 2 out of 11 (i.e. 18%) animals between day 15 and 30 post tumor cell inoculation. When looking at the frequency of CD⁸⁺ T cells that were able to bind gp70 tetramers, highest gp70-specific CD⁸⁺ T-cell frequencies were detectable in mCD137-3H3xmPD-L1-MPDL3280A treated animals (2.14%±1.52%). In comparison, gp70 tetramer⁺ CD8⁻ T-cell frequencies in mCD137-3H3xb12 (0.90%±0.46%), mPD-L1-MPDL3280Axb12 (0.94%±1.06%) and PBS-treated control animals (0.66%±0.49%) were considerably lower with only minimal differences between those three treatment modalities.

### Example 12: Binding of PD-L1 Antibodies or b12xPD-L1 Bispecific Antibodies to Tumor Cells

Binding of PD-L1 antibodies and b12xPD-L1 bispecific antibodies to human tumor cell lines MDA-MB-231 (breast adenocarcinoma; ATCC; Cat. no. HTB-26), PC-3 (prostate adenocarcinoma; ATCC; Cat. no. CRL-1435) and SK-MES-1 (lung squamous cell carcinoma; ATCC; Cat. no. HTB-58) was analyzed by flow cytometry.

Cells (3-5×10⁴ cells/well) were incubated in polystyrene 96-well round-bottom plates (Greiner bio-one, cat. no. 650101) with serial dilutions of antibodies (range 0.0001 to 10 μg/mL in 5-fold dilution steps) in 50 μL PBS/0.1% BSA/0.02% azide (FACS buffer) at 4° C. for 30 min. After washing twice in FACS buffer, cells were incubated with secondary antibody at 4° C. for 30 min. As a secondary antibody, R-Phycoerythrin (PE)-conjugated goat-anti-human IgG F(ab′)₂ (Cat. no. 109-116-098, Jackson ImmunoResearch Laboratories, Inc., West Grove, Pa.) diluted 1:500 in 50 μL FACS buffer, was used for all experiments. Next, cells were washed twice in FACS buffer, re-suspended in 20 μL FACS buffer and analyzed on an iQue screener (Intellicyt Corporation, USA). Binding curves were analyzed using non-linear regression (sigmoidal dose-response with variable slope) using GraphPad Prism V75.04 software (GraphPad Software, San Diego, Calif., USA).

Quantitative flow cytometry (QIFIKIT®, Dako; cat. no K0078) was performed as described (Poncelet and Carayon, 1985, J. Immunol. Meth. 85: 65-74) using MPDL3280A (heavy and light chain variable sequences set forth in SEQ ID NOs: 50 and 51, respectively), to quantify antigen density on the plasma membrane of MDA-MB-231, PC-3 and SK-MES-1 cells. It was determined that the cells lines have the following PD-L1 antigen density (ABC, antibody binding capacity):
MDA-MB-231: appr. 21,000 ABC/cell
PC-3: appr. 6,000 ABC/cell
SK-MES-1: appr. 30,000 ABC/cell

### Binding to MDA-MB-231 Cells

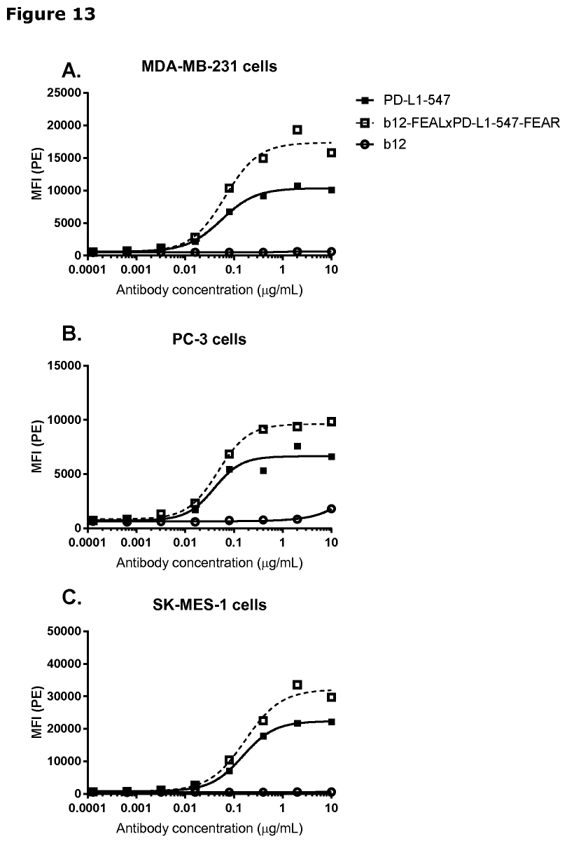

FIG. 13 (A) shows dose-dependent binding of b12-FEALxPD-L1-547-FEAR to MDA-MB-231 cells, with higher maximum binding than monospecific, bivalent PD-L1-547-FEAR.

### Binding to PC-3 Cells

FIG. 13 (B) shows dose-dependent binding of b12-FEALxPD-L1-547-FEAR to PC3 cells, with higher maximum binding than monospecific, bivalent PD-L1-547-FEAR.

### Binding to SK-MES-1 Cells

FIG. 13 (C) shows dose-dependent binding of b12-FEALxPD-L1-547-FEAR to SK-MES-1 cells, with higher maximum binding than monospecific, bivalent PD-L1-547-FEAR.

### Example 13: Non-Antigen-Specific t-Cell Proliferation Assay to Measure Effects of Bispecific Antibodies Binding to Pd-l1 and Cd137

A schematic representation of the anticipated mode of action of PD-L1xCD137 bispecific antibodies is shown in FIG. 6.

To measure induction of T-cell proliferation in polyclonally activated T cells, PBMCs were incubated with a sub-optimal concentration of anti-CD3 antibody (clone UCHT1), to activate T cells, combined with PD-L1-547-FEALxCD137-009-HC7LC2-FEAR bispecific antibody or control antibodies. Within the PBMC population, cells expressing PD-L1 can be bound by the PD-L1-specific arm of the bispecific antibody, whereas the T cells in the population can be bound by the CD137-specific arm. In this assay, T-cell proliferation is a measure for trans-activation of the T cells via the CD137-specific arm, induced by cross-linking with the PD-L1-expressing cells via the bispecific antibody and by blockade of PD-L1:PD-1 interaction, is measured as T-cell proliferation.

PBMCs were obtained from buffy coats of healthy donors (Transfusionszentrale, University Hospital, Mainz, Germany) using a Ficoll gradient (VWR, cat. no. 17-5446-02). PBMCs were labeled using 1.6 μM carboxyfluorescein succinimidyl ester (CFSE) (Thermo Fisher, cat. no. C34564) in PBS, according to the manufacturer's instructions. 75,000 CFSE-labeled PBMCs were seeded per well in a 96-well round-bottom plate (Sigma Aldrich, CLS3799-50EA) and incubated with a sub-optimal concentration of anti-CD3 antibody (R&D Systems, clone UCHT1, cat. no. MAB100; 0.03-0.1 μg/mL final concentration) that was pre-determined for each donor to induce sub-optimal T cell proliferation, and bispecific or control antibodies, in 150 μL IMDM GlutaMAX supplemented with 5% human AB serum, at 37° C., 5% CO₂, for four days. Proliferation of CD⁴⁺ and CD8⁺ T cells was analyzed by flow cytometry, essentially as described supra. 30 μL containing PE-labeled CD4 antibody (BD Biosciences, cat. no. 555347; 1:80 final dilution), PE-Cy7-labeled CD8a antibody (clone RPA-T8, eBioscience, cat. no. 25-0088-41; 1:80 final dilution), APC-labeled CD56 antibody (eBiosciences, cat. no. 17-0567; 1:80 final dilution) and 7-AAD (Beckman Coulter, cat. no. A07704; 1:80 final dilution) in FACS buffer was used to stain the cells and exclude CD56⁺ natural killer (NK) cells and 7-AAD⁺ dead cells from the analysis. Samples were measured on a BD FACSCanto II flow cytometer (BD Biosciences) as proliferation read-out. Detailed analyses of T-cell proliferation based on CFSE-peaks indicating cell divisions were made by FlowJo 10.4 software and exported expansion index values were used to plot dose-response curves in GraphPad Prism version 6.04 (GraphPad Software, Inc). The expansion index determines the fold-expansion of the overall culture; an expansion index of 2.0 represents a doubling of the cell count, whereas an expansion index of 1.0 represents no change of the overall cell count.

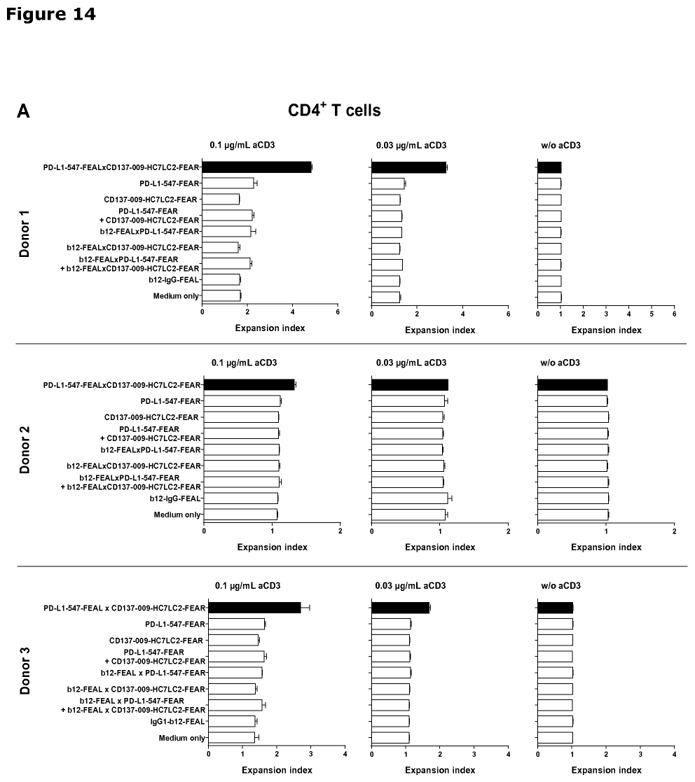

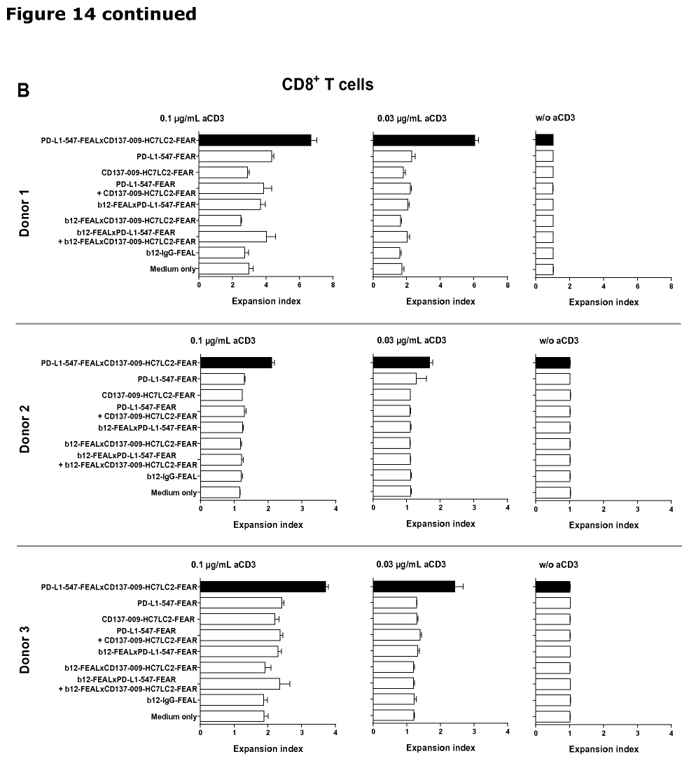

PBMCs from three different donors were analyzed testing two different anti-CD3 concentrations for stimulation and as control without anti-CD3. FIG. 14 shows that the bispecific antibody PD-L1-547-FEALxCD137-009-HC7LC2-FEAR induced a strong expansion of both CD⁴⁺ and CD⁸⁺ T cells. The monovalent CD137-control antibody, b12-FEALxCD137-009-HC7LC2-FEAR, having one irrelevant arm and the corresponding bivalent parental antibody CD137-009-HC7LC2-FEAR did not affect CD⁴⁺ (A) or CD8⁺ (B) T-cell proliferation when compared to incubation with the isotype control antibody b12 IgG. The monovalent PD-L1-control antibody as well as the bivalent parental antibody (b12-FEALxPD-L1-547-FEAR and PD-L1-547-FEAR, respectively) slightly enhanced T-cell proliferation compared to b12 IgG, only when the PBMC stimulation by anti-CD3 already resulted in a strong T cell activation (as observed by a higher expansion index in the medium only control group [see donor 1 at 0.1 μg/ml anti-CD3 stimulation]). A level of T-cell proliferation comparable to the monovalent and bivalent PD-L1 control antibodies was also detectable for the combined monovalent control antibodies (b12-FEALxCD137-009-HC7LC2-FEAR+b12-FEALxPD-L1-547-FEAR) and the combined corresponding parental antibodies (CD137-009-HC7LC2-FEAR+PD-L1-547-FEAR). However, the enhancement of proliferation induced by the bispecific PD-L1-547-FEALxCD137-009-HC7LC2-FEAR antibody was superior to both combined controls (monovalent and bivalent) (FIG. 14).

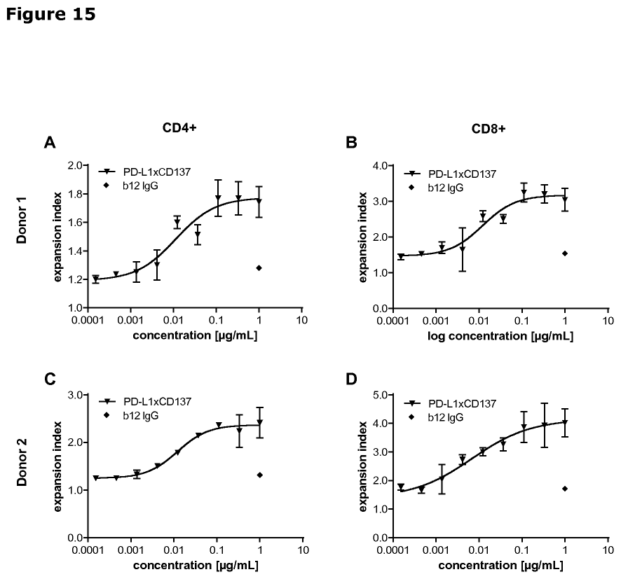

In another independent study EC₅₀ values for PD-L1-547-FEALxCD137-009-HC7LC2-FEAR were determined using PBMCs obtained from two donors, which were sub-optimally stimulated with 0.03 and 0.09 μg/mL anti-CD3. PD-L1-547-FEAL xCD137-009-HC7LC2-FEAR was assayed using serial dilutions starting at 1 μg/mL and ending at 0.15 ng/mL and b12-IgG-FEAL at 1 μg/mL was included as an isotype control antibody. For proliferation of CD⁴⁺ and CD8⁺ T-cells dose-response curves were generated (FIG. 15) and for CD⁸⁺ T-cell proliferation, EC₂₀, EC₅₀ and EC₉₀ values were determined as well, as shown in table 4.

TABLE 4
Determination of EC₂₀, EC₅₀ and EC₉₀-values of PD-L1-547-
FEALxCD137-009-HC7LC2-FEAR based on CD8⁺ T-cell expansion
data as measured by a non-antigen-specific T-cell proliferation
assay. Data shown are the values calculated based on the
four parameter logarithmic fits (FIG. 15).

| | | EC₅₀ | | Calc. | Calc. |
|---|---|---|---|---|---|
| | anti-CD3 | value | | EC₂₀ | EC₉₀ |
| Donor | [μg/ml] | [μg/ml] | Hill-Slope | [μg/ml] | [μg/ml] |
| 1 | 0.03 | 0.01218 | 1.134 | 0.00359 | 0.08455 |
| 2 | 0.09 | 0.00689 | 0.635 | 0.00078 | 0.21917 |

### Example 14: Antigen-Specific CD⁸⁺ T-Cell Proliferation Assay to Measure Cytokine Release Induced by Bispecific Antibodies Binding to PD-L1 and CD137

The induction of cytokine release by bispecific antibody PD-L1-547-FEALxCD137-009-HC7LC2-FEAR targeting PD-L1 and CD137 was measured in an antigen-specific assay, performed essentially as described in Example 7.

T cells were electroporated with 10 μg TCR α chain- and 10 μg β chain-encoding RNA, with or without 2 μg PD-1-encoding IVT RNA. Electroporated T cells were not CFSE-labeled (as described supra), but transferred into fresh IMDM medium (Life Technologies GmbH, cat. no. 12440-061) supplemented with 5% human AB serum, immediately after electroporation. iDCs were electroporated with 5 μg claudin-6 (CLDN6)-encoding RNA, as described supra. After O/N incubation, DCs were stained with Alexa647-conjugated CLDN6-specific antibody and T cells with anti-mouse TCR β chain antibody and with anti-human CD279 antibody, as described supra. 5,000 electroporated DCs were incubated with 50,000 electroporated T cells in the presence of different concentrations of PD-L1-547-FEALxCD137-009-HC7LC2-FEAR bispecific antibody or control antibody b12xIgG-FEAL in IMDM GlutaMAX supplemented with 5% human AB serum in a 96-well round-bottom plate. Following a 48-hour incubation period, the plates were centrifuged at 500×g for 5 min and the supernatant was carefully transferred from each well to a fresh 96-well round bottom plate and stored at −80° C. until cytokine analysis on the MSD® platform. The collected supernatants from the antigen-specific proliferation assay were analyzed for cytokine levels of 10 different cytokines by an MSD V-Plex Human Proinflammatory panel 1 (10-Plex) kit (Meso Scale Diagnostics, LLC., cat. no. K15049D-2) on a MESO QuickPlex SQ 120 instrument (Meso Scale Diagnostics, LLC., cat. no. R31QQ-3), according to the manufacturer's instructions.

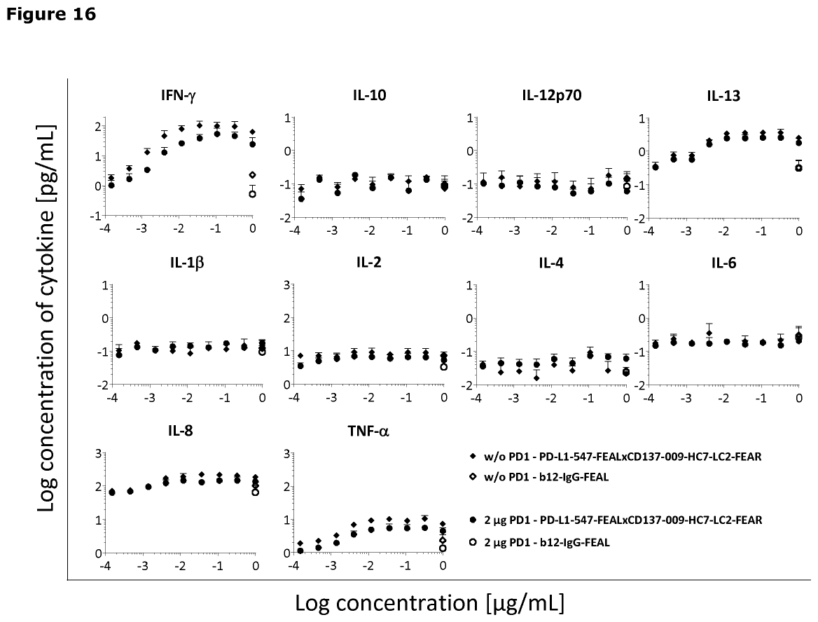

The addition of PD-L1-547-FEALxCD137-009-HC7LC2-FEAR led to a concentration-dependent increase in secretion of primarily IFN-γ, TNF-α, IL-13 and IL-8 (FIG. 16). Cytokine levels of all other cytokines (IL-10, IL-12p70, IL-1β, IL-2, IL-4, IL-6) were not elevated above those levels detected for co-cultures treated with control antibody b12-IgG-FEAL. When comparing T cell:DC co-cultures where T cells were not electroporated with PD-1 RNA to those where T cells were electroporated with 2 μg PD-1 RNA, slightly higher cytokine levels were detectable for co-cultures without PD-1 RNA electroporation. This was observed for both the PD-L1-547-FEALxCD137-009-FEAR dose response curve as well as for the b12-IgG-FEAL control antibody values.

### Example 15: Antigen-Unspecific In Vitro T-Cell Proliferation Assay to Measure Cytokine Release Induced by Bispecific Antibodies Binding to PD-L1 and CD137

Induction of cytokine release by the bispecific antibody PD-L1-547-FEALxCD137-009-HC7LC2-FEAR targeting PD-L1 and CD137 was measured in an antigen-unspecific in vitro T-cell proliferation assay, performed essentially as described supra (Example 14). The effect of trans-binding, i.e. simultaneous binding of both arms to its respective targets, on cytokine release of ten pro-inflammatory cytokines (IFN-γ, TNF-α, IL-13, IL-8, IL-10, IL-12p70, IL-1β, IL-2, IL-4, IL-6) was analyzed by a multiplex sandwich immunoassay of supernatants collected at 48 hours after antibody addition.

PBMCs were not CSFE labeled (as described supra), but were seeded immediately after isolation and only one concentration of anti-CD3 antibody (0.03 μg/mL final concentration) was used.

Following a 48-hour incubation period, the cells were collected by centrifugation at 500×g for 5 minutes and the supernatant was carefully transferred from each well to a fresh 96-well round bottom plate and stored at −80° C. until cytokine analysis on the MSD® platform. The collected supernatants were analyzed for cytokine levels of 10 different cytokines by an MSD V-Plex Human Proinflammatory panel 1 (10-Plex) kit (Meso Scale Diagnostics, LLC., cat. no. K15049D-2) on a MESO QuickPlex SQ 120 instrument (Meso Scale Diagnostics, LLC., cat. no. R31QQ-3), according to the manufacturer's instructions.

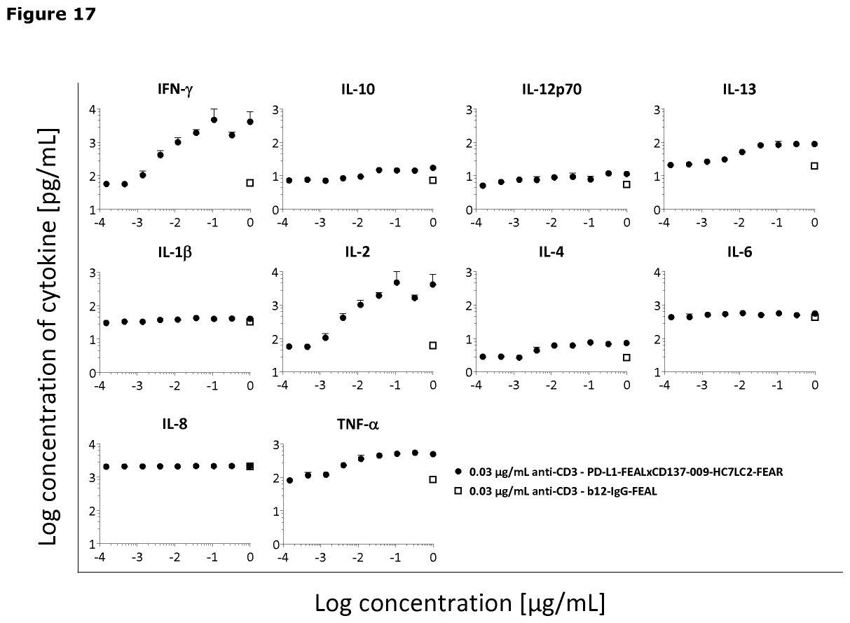

The addition of PD-L1-547-FEALxCD137-009-HC7LC2-FEAR induced a concentration-dependent increase in secretion of primarily IFN-γ, TNF-α, IL-2 and IL-13 (FIG. 17). A dose-response curve with only slightly elevated levels was also detectable for IL-10, IL-12p70 as well as IL-4. Cytokine levels of IL-1β, IL-6 and IL-8 remained at baseline levels and hence were comparable to those levels detected for co-cultures treated with control antibody b12-IgG-FEAL.

### Example 16: Antibody Formulation

Antibodies IgG1-7717-547-FEAL (7717b) and IgG1-CD137-009-HC7LC2-FEAR (7729a) were combined to the DuoBody® BisG1-7717-547-FEAL/CD137-009-HC7LC2-FEAR using the 2-MEA-induced Fab-arm exchange process described in Example 5. Following the exchange process the DuoBody was formulated at 20 mg/mL in 20 mM Histidine, 250 mM Sucrose at pH 5.5 with the addition of 0.02% PS80 or PS20. To verify the suitable characteristic of the formulation a study was conducted which evaluated the impact of pH, Excipient concentration and type of Surfactant. Table 5 below provides an overview of the formulations prepared, including three liquid formulations and one lyophilized formulation.

TABLE 5
Formulations

| | Dosage | Buffer | | Antibody | | Surfactant |
|---|---|---|---|---|---|---|
| Formulation | form | system | pH | (mg/ml) | Excipient 1 | (w/v) |
| F1 | Liquid | 20 mM | 5.5 | 20 | 250 mM | 0.02% PS80 |
| | | L-His/L-His•H₂O | | | Sucrose | |
| F2 | | | 5.5 | 20 | 250 mM | 0.02% PS20 |
| | | | | | Sucrose | |
| F3 | | | 6.0 | 20 | 250 mM | 0.02% PS80 |
| | | | | | Sucrose | |
| F4 | Lyo | | 5.5 | 50 | 250 mM | 0.02% PS80 |
| | | | | | Sucrose | |

At the beginning of the formulation stability study, each of the liquid formulations were split into two work streams. In one workstream, the liquid formulations were subjected to 5 freeze-thaw cycles consisting of freezing for 12 h at −65° C. following by thawing for 12 h at 25° C.

Samples were tested after the 5 freeze/thaw cycles with the same methods used for the second workstream which evaluated stability of the liquid formulation at time points 0, 1 and 2 months

### Visible Particles

Visible particle count was performed against a black background and against a white background at an illumination of a minimum intensity between 2000 and 3750 lux.

All formulations were practically free of visible particles (0-3 particles/ml) both at time 0 but only F1 and F2 after the freeze-thaw cycles. Thus, the samples in the F1 and F2 formulations were stable with regards to visible particles formation. Results are shown in Table 6.

### Turbidity

Turbidity testing was done by measurement against pharmacopoeial reference standard solutions using a turbidimeter. The result of the sample solution (in Nephelometric Turbidity Units (NTU)) was compared with the result of the closest reference solution. If the sample result was within [−10% to +10%], the respective reference solution's NTU value, the result was reported as equal to the reference solution.

All turbidity values were low. F1 showed the lowest turbidity with little change upon storage and under stressed conditions. Results are shown in Table 6.

### Sub-Visible Particles

Sub-visible particles after 5 freeze-thaw cycles were detected by the principle of light obscuration using a HIAC instrument. Particles of more than 2, 5, 10 or 25 micrometers were counted. Results are shown in Table 6.

All tested formulations only contained few sub-visible particles, in particular few particles over 10 or 25 micrometers.

### Size Exclusion Chromatography (SEC)

Size exclusion UPLC (SE-UPLC) was used to determine the amount of monomer, high molecular weight species (HMWS/aggregates) and low molecular weight species (LMWS/fragments) present in the samples. The main peak, HMWS and LMWS are expressed as a percentage of the relative peak area (%). Results are shown in Table 8.

The data showed that the total HMWS and LMWS were low for all formulation and that no significant increases of HMWS and LMWS were found after 5 cycles of freeze-thawing but increased aggregation was observed for stressed condition. There were no major differences between the formulations. Results are shown in Table 8.

Imaged Capillary Isoelectric Focusing (icIEF)

A drop in the main isoform at both accelerated and stressed conditions was observed. The loss appears to result in both acidic and basic variants, and there is a pH dependence as well, with more acidic variants created at the higher pH (F3) compared to other formulations. At recommended storage conditions no change is observed. Results are shown in Table 8.

### Reverse Phase Chromatography (RP-HPLC):

Under non-reducing conditions there is increase in main peak content at accelerated and stressed conditions. In reducing conditions minor changes are observed. Results are shown in Table 9.

### Capillary Electrophoresis Sodium Dodecyl Sulfate (CE-SDS)

The stability profile is very robust for this parameter for all samples tested. Results are shown in Table 10.

Overall the F1 formulation exhibited suitable characteristics for pharmaceutical uses.

TABLE 6
Determination of visible particles, turbidity and sub-visible particles,

| Sample | Visual inspection | | | | Color | Sub-visible particles | | | |
|---|---|---|---|---|---|---|---|---|---|
| Description | Seidenader | B/W | Particle | Turbidity | Series | (Cumulative counts/mL) | | | |
| Sample ID | (#/vial) | (#/vial) | classification | (NTU) | (BY) | ≥2 μm | ≥5 μm | ≥10 μm | ≥25 μm |
| F1, T0, 5° C. | PFVP | — | — | 5 | ≤BY7 | 49 | 19 | 10 | 1 |
| F2, T0, 5° C. | PFVP | — | — | 5 | ≤BY7 | 41 | 14 | 4 | 0 |
| F3, T0, 5° C. | PFVP | — | — | 6 | ≤BY7 | 33 | 12 | 4 | 0 |
| F1, 5 d, 25° C. | PFVP | — | — | 5 | ≤BY7 | 26 | 12 | 8 | 1 |
| F2, 5 d, 25° C. | PFVP | — | — | 5 | ≤BY7 | 58 | 23 | 13 | 3 |
| F3, 5 d, 25° C. | PFVP | — | — | 6 | ≤BY7 | 56 | 18 | 6 | 2 |
| F1, 5X, ≤−65 | PFVP | — | — | 5 | ≤BY7 | 22 | 13 | 5 | 1 |
| 12 h & 25 12 h ° C. | | | | | | | | | |
| F2, 5X, ≤−65 | PFVP | — | — | 5 | ≤BY7 | 33 | 19 | 13 | 1 |
| 12 h & 25 12 h ° C. | | | | | | | | | |
| F3, 5X, ≤−65 | Few | 2 | Extraneous fibers | 6 | ≤BY7 | 30 | 12 | 5 | 1 |
| 12 h & 25 12 h ° C. | | | | | | | | | |
| F1, T1M, 5° C. | PFVP | — | — | 5 | ≤BY7 | 143 | 33 | 6 | 0 |
| F2, T1M, 5° C. | Few | White | Spherical | 5 | ≤BY7 | 178 | 48 | 11 | 2 |
| F3, T1M, 5° C. | Few | 1 White | — | 6 | ≤BY7 | 149 | 38 | 3 | 0 |
| F1, T1M, 25° C. | Few | 5 | 2 extraneous fibers | 6 | ≤BY7 | 173 | 58 | 10 | 1 |
| | | | 3 spherical | | | | | | |
| F2, T1M, 25° C. | Few | 1 White | spherical | 6 | ≤BY7 | 201 | 71 | 16 | 0 |
| F3, T1M, 25° C. | Few | 1 White | Spherical | 6 | ≤BY7 | 121 | 54 | 17 | 1 |
| F1, T1M, 40° C. | Few | White | Spherical | 7 | ≤BY7 | 227 | 89 | 23 | 1 |
| F2, T1M, 40° C. | Few | White | Spherical + fibers | 8 | ≤BY6 | 156 | 55 | 8 | 0 |
| F3, T1M, 40° C. | Few | 1 White | Spherical | 8 | ≤BY6 | 82 | 41 | 15 | 1 |
| F1, T2M, 5° C. | PFVP | — | — | 6 | ≤BY7 | 36 | 8 | 1 | 0 |
| F2, T2M, 5° C. | Few | 1 | Spherical | 6 | ≤BY7 | 111 | 8 | 1 | 0 |
| F3, T2M, 5° C. | PFVP | — | — | 6 | ≤BY7 | 138 | 9 | 3 | 0 |
| F1, T2M, 25° C. | Few | 2 | Spherical | 6 | ≤BY7 | 183 | 36 | 5 | 0 |
| F2, T2M, 25° C. | PFVP | — | — | 6 | ≤BY7 | 48 | 8 | 2 | 1 |
| F3, T2M, 25° C. | Few | 1 | Extraneous fibers | 7 | ≤BY7 | 28 | 5 | 3 | 0 |
| F1, T2M, 40° C. | PFVP | — | — | 9 | ≤BY6 | 71 | 25 | 6 | 0 |
| F2, T2M, 40° C. | PFVP | — | — | 9 | ≤BY6 | 18 | 6 | 2 | 0 |
| F3, T2M, 40° C. | PFVP | — | — | 12 | ≤BY6 | 28 | 4 | 2 | 0 |

TABLE 7
Determination of osmolality, pH and protein content

| Sample Description | Osmolality | | Content |
|---|---|---|---|
| Sample ID | (mOsmol/kg) | pH | Mg/ml |
| F1, T0, 5° C. | 322 | 5.5 | 19.8 |
| F2, T0, 5° C. | 325 | 5.5 | 20.1 |
| F3, T0, 5° C. | 318 | 5.9 | 20.7 |
| F1, 5 d, 25° C. | N/A | 5.5 | 20.6 |
| F2, 5 d, 25° C. | N/A | 5.5 | 20.4 |
| F3, 5 d, 25° C. | N/A | 5.9 | 20.4 |
| F1, 5X, ≤−65 12 h & 25 12 h ° C. | N/A | 5.5 | 20.5 |
| F2, 5X, ≤−65 12 h & 25 12 h ° C. | N/A | 5.5 | 20.2 |
| F3, 5X, ≤−65 12 h & 25 12 h ° C. | N/A | 5.9 | 21.6 |
| F1, T1M, 5° C. | N/A | 5.5 | 20.3 |
| F2, T1M, 5° C. | N/A | 5.5 | 20.0 |
| F3, T1M, 5° C. | N/A | 5.9 | 20.2 |
| F1, T1M, 25° C. | N/A | 5.5 | 19.9 |
| F2, T1M, 25° C. | N/A | 5.5 | 20.0 |
| F3, T1M, 25° C. | N/A | 5.9 | 20.5 |
| F1, T1M, 40° C. | N/A | 5.5 | 20.1 |
| F2, T1M, 40° C. | N/A | 5.5 | 20.0 |
| F3, T1M, 40° C. | N/A | 5.9 | 20.3 |
| F1, T2M, 5° C. | N/A | 5.5 | 19.6 |
| F2, T2M, 5° C. | N/A | 5.5 | 19.6 |
| F3, T2M, 5° C. | N/A | 5.9 | 20.4 |
| F1, T2M, 25° C. | N/A | 5.5 | 20.1 |
| F2, T2M, 25° C. | N/A | 5.5 | 19.8 |
| F3, T2M, 25° C. | N/A | 5.9 | 20.1 |
| F1, T2M, 40° C. | N/A | 5.5 | 20.1 |
| F2, T2M, 40° C. | N/A | 5.5 | 20.2 |
| F3, T2M, 40° C. | N/A | 5.9 | 20.5 |

TABLE 8
Size exclusion chromatography (determination of the amounts of monomers, high
molecular weight species (HMW/aggregates) and low molecular weight species
(LMW/fragments)); CEX/iCE (determination of the appearance of basic and acidic
variants under valour conditions); surfactant content determination.

| | | | | | | | Surfactant |
|---|---|---|---|---|---|---|---|
| | | | | iCE | | | content |
| | Size exclusion chromatography | | | Acidic | | Basic | determination |
| Sample Description | HMW | Main Peak | LMW | regions | Main peak | regions | [Surfactant] |
| Sample ID | (% area) | (% area) | (% area) | (% area) | (% area) | (% area) | % |
| F1, T0, 5° C. | 1.1 | 98.7 | 0.2 | 37.1 | 57.3 | 5.5 | 0.02 |
| F2, T0, 5° C. | 1.0 | 98.7 | 0.3 | 37.7 | 56.7 | 5.6 | 0.02 |
| F3, T0, 5° C. | 1.1 | 98.7 | 0.3 | 37.3 | 57.4 | 5.4 | 0.02 |
| F1, 5 d, 25° C. | 1.1 | 98.6 | 0.3 | N/A | N/A | N/A | 0.02 |
| F2, 5 d, 25° C. | 1.1 | 98.6 | 0.3 | N/A | N/A | N/A | 0.02 |
| F3, 5 d, 25° C. | 1.2 | 98.6 | 0.3 | N/A | N/A | N/A | 0.02 |
| F1, 5X, ≤−65 | 1.3 | 98.5 | 0.3 | N/A | N/A | N/A | 0.02 |
| 12 h & 25 12 h ° C. | | | | | | | |
| F2, 5X, ≤−65 | 1.0 | 98.7 | 0.3 | N/A | N/A | N/A | 0.02 |
| 12 h & 25 12 h ° C. | | | | | | | |
| F3, 5X, ≤−65 | 1.1 | 98.7 | 0.3 | N/A | N/A | N/A | 0.02 |
| 12 h & 25 12 h ° C. | | | | | | | |
| F1, T1M, 5° C. | 1.1 | 98.6 | 0.3 | 36.9 | 56.9 | 6.1 | 0.02 |
| F2, T1M, 5° C. | 1.1 | 98.6 | 0.3 | 37.2 | 57.0 | 5.8 | 0.02 |
| F3, T1M, 5° C. | 1.1 | 98.6 | 0.3 | 36.7 | 57.9 | 5.4 | 0.02 |
| F1, T1M, 25° C. | 1.8 | 97.8 | 0.4 | 37.8 | 54.6 | 7.6 | 0.02 |
| F2, T1M, 25° C. | 1.8 | 97.8 | 0.5 | 38.0 | 54.5 | 7.5 | 0.02 |
| F3, T1M, 25° C. | 1.7 | 97.9 | 0.4 | 39.0 | 54.7 | 6.3 | 0.02 |
| F1, T1M, 40° C. | 5.7 | 92.3 | 2.0 | 54.4 | 34.4 | 11.2 | 0.02 |
| F2, T1M, 40° C. | 6.0 | 92.1 | 1.9 | 55.4 | 34.1 | 10.5 | 0.02 |
| F3, T1M, 40° C. | 4.9 | 93.4 | 1.7 | 56.0 | 35.6 | 8.4 | 0.02 |
| F1, T2M, 5° C. | 1.2 | 98.5 | 0.3 | 35.9 | 58.3 | 5.8 | 0.02 |
| F2, T2M, 5° C. | 1.2 | 98.5 | 0.3 | 36.1 | 57.9 | 6.0 | 0.02 |
| F3, T2M, 5° C. | 1.2 | 98.5 | 0.3 | 36.7 | 57.9 | 5.4 | 0.02 |
| F1, T2M, 25° C. | 2.3 | 97.0 | 0.7 | 36.9 | 52.6 | 7.5 | 0.02 |
| F2, T2M, 25° C. | 2.4 | 96.9 | 0.7 | 40.6 | 51.8 | 7.6 | 0.02 |
| F3, T2M, 25° C. | 2.3 | 97.1 | 0.7 | 41.8 | 51.6 | 6.6 | 0.02 |
| F1, T2M, 40° C. | 10.3 | 86.2 | 3.5 | 68.8 | 23.3 | 7.9 | 0.02 |
| F2, T2M, 40° C. | 10.5 | 86.0 | 3.5 | 70.2 | 23.8 | 6.0 | 0.02 |
| F3, T2M, 40° C. | 9.7 | 87.1 | 3.1 | 72.5 | 22.7 | 4.8 | 0.01 |

TABLE 9
Reverse phase-HPLC

| | RP-HPLC | | | | | | |
|---|---|---|---|---|---|---|---|
| | | | | Reduced | | | |
| | Non-reduced | | | | Peak 2 | | |
| | Main | | | Peak 1 | (HC X1 + LC | Peak 3 | |
| Sample Description | Peak | Pre Peak | Post Peak | (LC X1) | B1) | (HC B1) | Total |
| Sample ID | (% area) | (% area) | (% area) | (% area) | (% area) | (% area) | (% area) |
| F1, T0, 5° C. | 61.6 | 2.1 | 36.4 | 23.6 | 47.2 | 21.2 | 92.0 |
| F2, T0, 5° C. | 63.3 | 1.9 | 34.8 | 23.7 | 47.2 | 21.3 | 92.2 |
| F3, T0, 5° C. | 59.6 | 1.8 | 38.7 | 23.7 | 47.2 | 21.4 | 92.3 |
| F1, 5 d, 25° C. | n/a | n/a | n/a | n/a | n/a | n/a | n/a |
| F2, 5 d, 25° C. | n/a | n/a | n/a | n/a | n/a | n/a | n/a |
| F3, 5 d, 25° C. | n/a | n/a | n/a | n/a | n/a | n/a | n/a |
| F1, 5X, ≤−65 | n/a | n/a | n/a | n/a | n/a | n/a | n/a |
| 12 h & 25 12 h ° C. | | | | | | | |
| F2, 5X, ≤−65 | n/a | n/a | n/a | n/a | n/a | n/a | n/a |
| 12 h & 25 12 h ° C. | | | | | | | |
| F3, 5X, ≤−65 | n/a | n/a | n/a | n/a | n/a | n/a | n/a |
| 12 h & 25 12 h ° C. | | | | | | | |
| F1, T1M, 5° C. | 65.2 | 1.4 | 33.4 | 23.3 | 48.1 | 19.5 | 90.9 |
| F2, T1M, 5° C. | 64.8 | 1.4 | 33.8 | 23.4 | 48.7 | 19.7 | 91.8 |
| F3, T1M, 5° C. | 64.3 | 1.3 | 34.4 | 23.8 | 48.2 | 19.3 | 91.3 |
| F1, T1M, 25° C. | 70.2 | 1.2 | 28.6 | 23.2 | 48.6 | 20.2 | 92.0 |
| F2, T1M, 25° C. | 73.0 | 1.3 | 25.7 | 23.0 | 48.3 | 19.8 | 91.1 |
| F3, T1M, 25° C. | 70.7 | 1.0 | 28.3 | 22.9 | 48.4 | 20.4 | 91.7 |
| F1, T1M, 40° C. | 71.0 | 1.6 | 27.4 | 21.8 | 45.8 | 20.7 | 88.3 |
| F2, T1M, 40° C. | 70.3 | 1.6 | 28.2 | 21.6 | 45.3 | 20.5 | 87.4 |
| F3, T1M, 40° C. | 70.8 | 1.5 | 27.7 | 21.2 | 45.8 | 20.2 | 87.2 |
| F1, T2M, 5° C. | 66.1 | 1.7 | 32.1 | 22.6 | 48.1 | 21.4 | 92.1 |
| F2, T2M, 5° C. | 65.7 | 1.6 | 32.7 | 22.5 | 48.2 | 21.4 | 92.1 |
| F3, T2M, 5° C. | 64.9 | 1.6 | 33.5 | 22.6 | 48.3 | 21.5 | 92.3 |
| F1, T2M, 25° C. | 73.2 | 1.7 | 25.1 | 21.9 | 46.8 | 21.3 | 90.0 |
| F2, T2M, 25° C. | 72.0 | 1.7 | 26.4 | 21.6 | 46.6 | 22.0 | 90.2 |
| F3, T2M, 25° C. | 73.4 | 1.5 | 25.1 | 21.4 | 46.3 | 21.5 | 89.2 |
| F1, T2M, 40° C. | 66.7 | 2.4 | 31.0 | 18.7 | 42.5 | 20.4 | 81.7 |
| F2, T2M, 40° C. | 65.6 | 3.0 | 31.4 | 19.3 | 42.0 | 20.2 | 81.5 |
| F3, T2M, 40° C. | 67.3 | 2.9 | 29.8 | 18.0 | 41.3 | 20.1 | 79.4 |

TABLE 10
Purity determination by Capillary Electrophoresis-SDS (CE-SDS)

| | CE-SDS (Caliper) | | | | |
|---|---|---|---|---|---|
| | Non-reduced | Reduced | | | |
| Sample Description | Intact IgG | LC1 | LC2 | HC | Total |
| Sample ID | (% area) | (% area) | (% area) | (% area) | (% area) |
| F1, T0, 5° C. | 95.8 | 11.7 | 10.1 | 78.0 | 99.8 |
| F2, T0, 5° C. | 95.8 | 11.7 | 10.1 | 78.0 | 99.7 |
| F3, T0, 5° C. | 95.7 | 11.7 | 10.1 | 78.0 | 99.7 |
| F1, 5 d, 25° C. | 95.8 | 11.7 | 10.0 | 78.0 | 99.7 |
| F2, 5 d, 25° C. | 96.0 | 11.7 | 10.0 | 78.1 | 99.7 |
| F3, 5 d, 25° C. | 95.7 | 11.6 | 10.0 | 78.1 | 99.7 |
| F1, 5X, ≤−65 12 h & 25 12 h ° C. | 95.7 | 11.6 | 9.9 | 78.2 | 99.7 |
| F2, 5X, ≤−65 12 h & 25 12 h ° C. | 95.7 | 11.6 | 9.9 | 78.2 | 99.7 |
| F3, 5X, ≤−65 12 h & 25 12 h ° C. | 95.6 | 12.0 | 9.7 | 77.5 | 99.2 |
| F1, T1M, 5° C. | 95.7 | 12.5 | 10.8 | 76.4 | 99.7 |
| F2, T1M, 5° C. | 95.6 | 12.5 | 10.6 | 76.6 | 99.8 |
| F3, T1M, 5° C. | 95.9 | 12.4 | 10.6 | 76.7 | 99.7 |
| F1, T1M, 25° C. | 95.8 | 12.7 | 10.7 | 76.4 | 99.7 |
| F2, T1M, 25° C. | 96.2 | 12.6 | 10.8 | 76.3 | 99.7 |
| F3, T1M, 25° C. | 96.2 | 12.6 | 10.6 | 76.5 | 99.7 |
| F1, T1M, 40° C. | 95.4 | 13.3 | 10.6 | 75.0 | 98.9 |
| F2, T1M, 40° C. | 95.0 | 13.3 | 10.7 | 74.9 | 98.9 |
| F3, T1M, 40° C. | 95.0 | 13.3 | 10.4 | 74.5 | 98.3 |
| F1, T2M, 5° C. | 98.3 | 11.7 | 10.2 | 77.9 | 99.9 |
| F2, T2M, 5° C. | 98.4 | 11.7 | 10.2 | 78.0 | 99.9 |
| F3, T2M, 5° C. | 98.4 | 11.9 | 10.6 | 77.5 | 100 |
| F1, T2M, 25° C. | 98.6 | 11.9 | 10.1 | 77.8 | 99.8 |
| F2, T2M, 25° C. | 97.6 | 12.0 | 10.2 | 77.7 | 99.9 |
| F3, T2M, 25° C. | 98.0 | 11.9 | 9.9 | 78.1 | 99.9 |
| F1, T2M, 40° C. | 98.0 | 13.4 | 9.6 | 75.8 | 98.8 |
| F2, T2M, 40° C. | 97.8 | 13.4 | 9.8 | 75.6 | 98.8 |
| F3, T2M, 40° C. | 98.0 | 13.6 | 9.7 | 75.2 | 98.5 |

### Example 17: Antibody Formulation; Stability Study

Long-term (12-month) stability studies were conducted on DuoBody BisG1-7717-547-FEAL/CD137-009-HC7LC2-FEAR, batch 6371-16 (production date: 18 May 2018).

Storage conditions and testing intervals for the stability samples of DuoBody® BisG1-7717-547-FEAL/CD137-009-HC7LC2-FEAR, batch 6371-16 are indicated in Table 11.

TABLE 11
Storage conditions and pull intervals

| | Interval (months)/amount per pull | | | | | | |
|---|---|---|---|---|---|---|---|
| Storage Conditions | 0 | 1 | 2 | 3 | 6 | 9 | 12 |
| ≤−65° C. (−80° C. ± 10° C.) | 20 mL | 20 mL | 20 mL | 20 mL | 20 mL | 20 mL | 20 mL |
| 5° C. ± 3° C. | | 20 mL | 20 mL | — | — | — | — |
| 40° C. ± 2° C./75% rH | | 20 mL | 20 mL | 20 mL | 20 mL | — | — |

rH means “relative humidity”

An appropriate representative sample of DuoBody® BisG1-7717-547-FEAL/CD137-009-HC7LC2-FEAR, batch 6371-16 was taken from the bulk container. For each storage condition and time interval, aliquots of 20 mL DuoBody® BisG1-7717-547-FEAL/CD137-009-HC7LC2-FEAR, were stored as described in Table 12, simulating the shipping and storage containers.

TABLE 12
Packaging material

| Packaging | Packaging Material |
|---|---|
| Simulated primary Packaging | 50 mL sample bag Allegro 2D standard |
| | system with AdvantaPure tubing system |
| Simulated secondary Packaging | The bags were packed in 2 zipper PE |
| | bags for safety reasons |

For each pull point and storage condition, one bag was removed from the appropriate storage chamber to accomplish the tests.

### Appearance and Color (European Pharmacopoeia Color Visual Liquid Color Scale):

No changes were observed for the appearance and color for storage conditions ≤−65° C. and 5° C. For storage condition 40° C./75% rH the color had changed from ≤BY7 to ≤BY5 after 6 months storage.

### Opalescence:

The opalescence testing was done by measurement against pharmacopoeial reference standard solutions using a turbidimeter. The result of the sample solution (in Nephelometric Turbidity Units (NTU)) was compared with the result of the closest reference solution. If the sample result is within [−10% to +10%] the respective reference solution's NTU value, the result is reported as equal to the reference solution. No significant changes were observed for the opalescence. Results ranged from <Ref. II to <Ref. III.

pH:

The pH ranged between 5.4 and 5.6 and was well within the specified range of 5.2 to 5.8.

### Protein Concentration by UV280:

The protein concentration ranged between 20.1 and 20.6 mg/ml for storage conditions −65° C. and 5° C., which was well within the specified range of 18.0 to 22.0 mg/ml. For storage condition 40° C./75% rH results ranged between 19.5 and 22.9 mg/ml. The increase in protein concentration was possibly caused by evaporation of solvent.

### Purity by Size Exclusion Chromatography (SEC)-HPLC:

A shift from the main peak to the low molecular weight (LMW) and high molecular weight (HMW) forms was observed for storage condition 40° C./75% rH. The main peak was decreased from 99.1% area to 71.1% area after 6 months storage. For storage condition 5° C., a slight but not significant decrease of the main peak was observed after 2 months storage. No significant change was observed for storage condition ≤−65° C. after 12 months and all results met the defined specification.

### Purity by Hydrophobic Interaction Chromatography (HIC)-HPLC:

No significant changes were observed in antibody purity. The variations in antibody purity reflect analytical variation. No Homodimer PD-L1 was detected.

Charge Heterogeneity by Imaged Capillary Isoelectric Focusing (icIEF):

A shift from the main peak to the acidic species was observed for storage condition 40° C./75% rH. The main peak was decreased from 58.3% area to 5.7% area after 6 months storage. No significant changes were observed for storage conditions ≤−65° C. and 5° C.

### Purity by Capillary Electrophoresis (CE)-SDS:

Apart from the samples stored at 40° C./75% rH, all samples were comparable to the reference. A decreasing trend was observed for the purity under reduced and non-reduced conditions for storage 40° C./75% rH. The intact IgG, non-reduced, was decreased from 94.9 cor. % area to 77.0 cor. % area and the sum of HC and LC, reduced, was decreased from 99.0 cor. % area to 87.4 cor. % area after 6 months storage. No significant changes were observed for storage conditions ≤−65° C. and 5° C.

### CONCLUSION

The stability data showed that DuoBody BisG1-7717-547-FEAL/CD137-009-HC7LC2-FEAR is stable for 12 months when stored at −65° C. and for 2 months when stored at 5° C. in the undamaged original packaging. For storage condition 40° C./75% rH, results from SEC-HPLC, icIEF and CE-SDS showed significant degradation of DuoBody® BisG1-7717-547-FEAL/CD137-009-HC7LC2-FEAR after 1 month. The stability data for DuoBody® BisG1-7717-547-FEAL/CD137-009-HC7LC2-FEARbatch 6371-16 confirmed the defined shelf life of 365 days when the material is stored not above ≤−65° C. in the undamaged origin packaging.

TABLE 13
DuoBody ® BisG1-7717-547-FEAL/CD137-009-HC7LC2-FEAR, Batch 6371-16, Sample Storage Condition: ≤−65° C.

| | Component | Time points (Months) | | | | | | |
|---|---|---|---|---|---|---|---|---|
| Method/Assay | name | 0 | 1 | 2 | 3 | 6 | 9 | 12 |
| Appearance and | Appearance | Liquid | Liquid | Liquid | Liquid | Liquid | Liquid | Liquid |
| color | Color | ≤BY7 | ≤BY7 | ≤BY7 | ≤BY7 | ≤BY7 | ≤BY7 | ≤BY7 |
| Opalescence | Opalescence | =Ref. II | <Ref. III | <Ref. III | =Ref. II | =Ref. II | <Ref. II | =Ref. II |
| pH | pH (USP | 5.5 | 5.5 | 5.5 | 5.5 | 5.5 | 5.5 | 5.6 |
| | <791>) | | | | | | | |
| UV | Protein | 20.6 | 20.2 | 20.2 | 20.1 | 20.1 | 20.3 | 20.3 |
| absorption | concentration | | | | | | | |
| | (UV280, | | | | | | | |
| | Solo-VPE) | | | | | | | |
| SEC_HPLC | Purity, Main | 99.1 | 98.9 | 98.9 | 98.9 | 99.1 | 98.9 | 99.0 |
| | Peak | | | | | | | |
| | Purity, HMW | 0.8 | 0.9 | 0.9 | 0.9 | 0.8 | 0.9 | 0.9 |
| | Forms | | | | | | | |
| | Purity, LMW | 0.1 | 0.2 | 0.1 | 0.2 | 0.2 | 0.1 | 0.1 |
| | Forms | | | | | | | |
| HIC-HPLC | Purity, | 98.6 | 98.9 | 98.9 | 99.0 | 98.4 | 99.2 | 99.1 |
| | DuoBody | | | | | | | |
| | Purity, | 1.4 | 1.1 | 1.1 | 1.0 | 1.6 | 0.8 | 0.9 |
| | Homodimer | | | | | | | |
| | 4-1BB | | | | | | | |
| | Purity, | 0.0 | 0.0 | 0.0 | 0.0 | 0.0 | 0.0 | 0.0 |
| | Homodimer | | | | | | | |
| | PD-L1 | | | | | | | |
| icIEF | Charge | 58.3 | 58.0 | 57.9 | 58.3 | 57.5 | 57.8 | 57.5 |
| | Heterogeneity, | | | | | | | |
| | Main peak | | | | | | | |
| | Charge | 36.5 | 36.6 | 36.5 | 36.2 | 36.8 | 36.7 | 36.7 |
| | Heterogeneity, | | | | | | | |
| | acidic reg. | | | | | | | |
| | Charge | 5.2 | 5.4 | 5.6 | 5.5 | 5.7 | 5.4 | 5.8 |
| | Heterogeneity, | | | | | | | |
| | basic reg. | | | | | | | |
| CE-SDS | Purity, intact | 94.9 | 95.2 | 95.4 | 95.4 | 95.2 | 95.2 | 95.2 |
| | igG, non-red | | | | | | | |
| | ID comparable | Comparable | Comparable | Comparable | Comparable | Comparable | Comparable | Comparable |
| | to ref., | to reference | to reference | to reference | to reference | to reference | to reference | to reference |
| | non-red. | | | | | | | |
| | Purity, | 99.0 | 99.1 | 98.9 | 99.1 | 99.0 | 98.9 | 98.9 |
| | HC + LC, | | | | | | | |
| | red. | | | | | | | |
| | Purity, HC, | 65.0 | 65.3 | 64.3 | 65.4 | 65.4 | 65.9 | 64.6 |
| | red. | | | | | | | |
| | Purity, LC1, | 16.5 | 16.4 | 17.2 | 17.3 | 17.2 | 17.0 | 17.6 |
| | red. | | | | | | | |
| | Purity, LC2, | 17.4 | 17.3 | 17.3 | 17.3 | 17.2 | 17.0 | 17.6 |
| | red. | | | | | | | |
| | ID comparable | Comparable | Comparable | Comparable | Comparable | Comparable | Comparable | Comparable |
| | to ref., red. | to reference | to reference | to reference | to reference | to reference | to reference | to reference |
| | Purity, | N/A | 33.7 | 34.5 | 33.6 | 33.6 | 33.0 | 34.3 |
| | LC1 + LC2, | | | | | | | |
| | red. (calc.) | | | | | | | |

1. A pharmaceutical formulation comprising
a. a binding agent comprising a first antigen-binding region binding to human CD137 (4-1BB) and a second antigen-binding region binding to human PD-L1 (CD274),
the first antigen biding region comprising a first heavy chain variable region (VH) comprising the three complementarity determining regions, CDR1, CDR2, and CDR3, present within the amino acid sequence set forth in SEQ ID NO: 15, and a first light chain variable region (VL) comprising the three complementarity determining regions, CDR1, CDR2, and CDR3, present within the amino acid sequence set forth in SEQ ID NO: 16, and
the second antigen-binding region comprising a second heavy chain variable region (VH) comprising the three complementarity determining regions, CDR1, CDR2, and CDR3, present within the amino acid sequence set forth in SEQ ID NO: 17, and a second light chain variable region (VL) comprising the three complementarity determining regions, CDR1, CDR2, and CDR3, present within the amino acid sequence set forth in SEQ ID NO: 21;
b. a histidine buffer,
c. about 100 to about 400 mM of a sugar, and
d. about 0.001 to about 0.1% (w/v) non-ionic surfactant;
and having a pH between about 4.5 and about 6.5.

2. The pharmaceutical formulation according to claim 1, said formulation comprising 1 to 100 mM histidine, such as 5 to 100 mM, 10 to 100 mM, 15 to 100 mM, 5 to 90 mM, 5 to 80 mM, 5 to 70 mM, 5 to 60 mM, 5 to 50 mM, 5 to 40 mM, 5 to 30 mM, 10 to 90 mM, 10 to 80 mM, 10 to 70 mM, 10 to 60 mM, 10 to 50 mM, 10 to 40 mM, 10 to 30 mM, 15 to 90 mM, 15 to 80 mM, 15 to 70 mM, 15 to 60 mM, 15 to 50 mM, 15 to 40 mM, 15 to 30 mM or 15 to 20 mM histidine.

3. The pharmaceutical formulation according to any one of the preceding claims, said formulation comprising about 20 mM Histidine, such as 20 mM Histidine.

4. The pharmaceutical formulation according to any one of the preceding claims, said pharmaceutical formulation comprising 100 to 400 mM sugar, such as 125 to 400 mM, 150 to 400 mM, 150 to 400 mM, 175 to 400 mM, 200 to 400 mM, 225 to 400 mM, 100 to 375 mM, 100 to 350 mM, 100 to 325 mM, 100 to 300 mM, 125 to 375 mM, 125 to 350 mM, 125 to 325 mM, 125 to 300 mM, 125 to 275 mM, 150 to 375 mM, 150 to 350 mM, 150 to 325 mM, 150 to 300 mM, 150 to 275 mM, 175 to 375 mM, 175 to 350 mM, 175 to 325 mM, 175 to 300 mM, 175 to 275 mM, 200 to 375 mM 1, 200 to 350 mM 1, 200 to 325 mM, 200 to 300 mM, 200 to 275 mM, 225 to 375 mM, 225 to 350 mM, 225 to 325 mM, 225 to 300 mM, or such as 225 to 275 mM sugar.

5. The pharmaceutical formulation according to any one of the preceding claims, said formulation comprising about 250 mM sugar, such as 250 mM sugar.

6. The pharmaceutical formulation according to any one of the preceding claims, wherein the sugar is sucrose.

7. The pharmaceutical formulation according to any one of the preceding claims, said pharmaceutical formulation comprising 0.005 to 0.1% (w/v) non-ionic surfactant, such as 0.01 to 0.1% (w/v), 0.015 to 0.1% (w/v), 0.001 to 0.09% (w/v), 0.001 to 0.08% (w/v), 0.001 to 0.07% (w/v), 0.001 to 0.06% (w/v), 0.001 to 0.05% (w/v), 0.001 to 0.04% (w/v), 0.001 to 0.02% (w/v), 0.005 to 0.1% (w/v), 0.005 to 0.09% (w/v), 0.005 to 0.08% (w/v), 0.005 to 0.07% (w/v), 0.005 to 0.06% (w/v), 0.005 to 0.05% (w/v), 0.005 to 0.04% (w/v), 0.005 to 0.03% (w/v), 0.005 to 0.02% (w/v), 0.01 to 0.09% (w/v), 0.01 to 0.08% (w/v), 0.01 to 0.07% (w/v), 0.01 to 0.06% (w/v), 0.01 to 0.05% (w/v), 0.01 to 0.04% (w/v), 0.01 to 0.03% (w/v), 0.01 to 0.02% (w/v), 0.015 to 0.09% (w/v), 0.015 to 0.08% (w/v), 0.015 to 0.07% (w/v), 0.015 to 0.06% (w/v), 0.015 to 0.05% (w/v), 0.015 to 0.04% (w/v), 0.015 to 0.03% (w/v), or such as 0.015 to 0.02% (w/v) non-ionic surfactant.

8. The pharmaceutical formulation according to any one of the preceding claims, said formulation comprising about 0.02% (w/v) non-ionic surfactant, such as 0.02% (w/v) non-ionic surfactant.

9. The pharmaceutical formulation according to any of the preceding claims, wherein the non-ionic surfactant is 2-[2-[3,4-bis(2-hydroxyethoxy)oxolan-2-yl]-2-(2-hydroxyethoxy)ethoxy]ethyl (E)-octadec-9-enoate (Polyoxyethylene (20) sorbitan monooleate; Polysorbate 80) or 2-[2-[3,4-bis(2-hydroxyethoxy)oxolan-2-yl]-2-(2-hydroxyethoxy)ethoxy]ethyl dodecanoate (Polyoxyethylene (20) sorbitan monolaurate; Polysorbate 20).

10. The pharmaceutical formulation according to any one of the preceding claims, having a pH between 4.5 and 6.5, such as between 4.7 and 6.5, e.g. between 4.9 and 6.5, between 5.1 and 6.5, between 5.3 and 6.5, between 4.5 and 6.3, between 4.7 and 6.1, between 4.7 and 5.9, between 4.7 and 5.7, between 5.1 and 6.3, between 4.7 and 6.1, between 4.7 and 5.9, between 4.7 and 5.7, between 4.9 and 6.3, between 4.9 and 6.1, between 4.9 and 5.9, between 4.9 and 5.7, between 5.1 and 6.3, between 5.1 and 6.1, between 5.1 and 5.9, between 5.1 and 5.7, between 5.3 and 6.3, between 5.3 and 6.1, between 5.3 and 5.9, such as between 5.3 and 5.7.

11. The pharmaceutical formulation according to any one of the preceding claims, having a pH, which is about 5.5, such as a pH of 5.5.

12. The pharmaceutical formulation according to any one of the preceding claims, comprising 5 to 200 mg/mL of the binding agent, such as 10 to 200 mg/mL, 20 to 200 mg/mL, 40 to 200 mg/mL, 60 to 200 mg/mL, 80 to 200 mg/mL, 100 to 200 mg/mL, 120 to 200 mg/mL, 150 to 200 mg/mL, 5 to 150 mg/mL, 10 to 150 mg/mL, 20 to 150 mg/mL, 40 to 150 mg/mL, 60 to 150 mg/mL, 80 to 150 mg/mL, 100 to 150 mg/mL, 5 to 130 mg/mL, 10 to 130 mg/mL, 20 to 130 mg/mL, 40 to 130 mg/mL, 60 to 130 mg/mL, 80 to 130 mg/mL, 100 to 130 mg/mL, 5 to 100 mg/mL, 10 to 100 mg/mL, 15 to 100 mg/mL, 20 to 100 mg/mL, 30 to 100 mg/mL, 40 to 100 mg/mL, 50 to 100 mg/mL, 60 to 100 mg/mL, 5 to 80 mg/mL, 5 to 60 mg/mL, 5 to 50 mg/mL, 5 to 40 mg/mL, 5 to 30 mg/mL, 5 to 20 mg/mL, 10 to 80 mg/mL, 10 to 60 mg/mL, 10 to 50 mg/mL, 10 to 40 mg/mL, 10 to 30 mg/mL, 15 to 80 mg/mL, 15 to 60 mg/mL, 15 to 40 mg/mL, or such as 15 to 25 mg/mL of the binding agent.

13. The pharmaceutical formulation according to any one of the preceding claims, comprising about 20 mg/mL of the binding agent, such as 20 mg/mL of the binding agent.

14. The pharmaceutical formulation according to any one of the preceding claims, wherein the formulation comprises
i) about 20 mg/mL of the binding agent, such as about 40 mg/mL, about 60 mg/mL, about 80 mg/mL, about 100 mg/mL, about 120 mg/mL, or about 140 mg/mL, and
ii) about 20 mM Histidine, about 250 mM sugar, and about 0.02% (w/v) non-ionic surfactant and has a pH about 5.5.

15. The pharmaceutical formulation according to any one of the preceding claims, wherein the formulation comprises
i) 20 mg/mL of the binding agent such as 40 mg/mL, 60 mg/mL, 80 mg/mL, 100 mg/mL, 120 mg/mL, or 140 mg/mL, and
ii) 20 mM Histidine, 250 mM sugar, and 0.02% (w/v) non-ionic surfactant and has a pH of 5.5.

16. The pharmaceutical formulation according to any one of the preceding claims, wherein the formulation is essentially free of visible particles after having been subjected to 5 freeze-thaw cycles consisting of freezing for 12 h at −65° C. following by thawing for 12 h at 25° C., as determined by visible particle count performed against a black background and against a white background at an illumination of an intensity between 2000 and 3750 lux.

17. The pharmaceutical formulation according to any one of the preceding claims, wherein the binding agent is an antibody, such as a bispecific antibody.

18. The pharmaceutical formulation according to any one of the preceding claims, wherein each variable region comprises three complementarity determining regions, CDR1, CDR2, and CDR3, and four framework regions, FR1, FR2, FR3, and FR4.

19. The pharmaceutical formulation according to claim 18, wherein said complementarity determining regions and said framework regions are arranged from amino-terminus to carboxy-terminus in the following order: FR1, CDR1, FR2, CDR2, FR3, CDR3, FR4.

20. The pharmaceutical formulation according to any one of the preceding claims, wherein
the first antigen biding region comprises a first heavy chain variable region (VH) comprising the CDR1, CDR2, and CDR3 sequences set forth in: SEQ ID NO: 9, 10, 11, respectively, and a first light chain variable region (VL) comprising the CDR1, CDR2, and CDR3 sequences as set forth in: SEQ ID NO: 13, GAS, and SEQ ID NO: 14, respectively, and
the second antigen-binding region comprises a second heavy chain variable region (VH) comprising the CDR1, CDR2, and CDR3 sequences set forth in: SEQ ID NO: 18, 19 and 20 respectively, and a second light chain variable region (VL) comprising the CDR1, CDR2, and CDR3 sequences set forth in: SEQ ID NO: 22, DDN and SEQ ID NO: 23, respectively.

21. The pharmaceutical formulation according to any one of the preceding claims, wherein
the first antigen biding region comprises a first heavy chain variable region (VH) having at least 70%, at least 75%, at least 80%, at least 85%, at least 90%, at least 95%, at least 97%, at least 99%, or 100% sequence identity to the sequence set forth in SEQ ID NO: 15; and a first light chain variable region (VL) having at least 70%, at least 75%, at least 80%, at least 85%, at least 90%, at least 95%, at least 97%, at least 99%, or 100% sequence identity to the sequence set forth in SEQ ID NO: 16; and
the second antigen-binding region comprises a second heavy chain variable region (VH) having at least 70%, at least 75%, at least 80%, at least 85%, at least 90%, at least 95%, at least 97%, at least 99%, or 100% sequence identity to the sequence set forth in SEQ ID NO: 17; and a second light chain variable region (VL) having at least 70%, at least 75%, at least 80%, at least 85%, at least 90%, at least 95%, at least 97%, at least 99%, or 100% sequence identity to the sequence set forth in SEQ ID NO: 21.

22. The pharmaceutical formulation according to any one of the preceding claims, wherein
the first antigen biding region comprises a first heavy chain variable region (VH) comprising a CDR1, CDR2, and CDR3 sequence, as set forth in: SEQ ID NO: 9, 10 and 11, respectively, the first heavy chain variable region having at least 70%, at least 75%, at least 80%, at least 85%, at least 90%, at least 95%, at least 97%, at least 99%, or 100% sequence identity to the sequence set forth in SEQ ID NO: 15; and a first light chain variable region (VL) comprising a CDR1, CDR2, and CDR3 sequence, as set forth in: SEQ ID NO: 13, GAS, and SEQ ID NO: 14, respectively, the first light chain variable region having at least 70%, at least 75%, at least 80%, at least 85%, at least 90%, at least 95%, at least 97%, at least 99%, or 100% sequence identity to the sequence set forth in SEQ ID NO: 16, and
the second antigen-binding region comprises a second heavy chain variable region (VH) comprising a CDR1, CDR2, and CDR3 sequence, as set forth in: SEQ ID NO: 18, 19 and 20, respectively, the second heavy chain variable region having at least 70%, at least 75%, at least 80%, at least 85%, at least 90%, at least 95%, at least 97%, at least 99%, or 100% sequence identity to the sequence set forth in SEQ ID NO: 17; and a second light chain variable region (VL) comprising a CDR1, CDR2, and CDR3 sequence, as set forth in: SEQ ID NO: 22, DDN, 23, respectively, the second light chain variable region having at least 70%, at least 75%, at least 80%, at least 85%, at least 90%, at least 95%, at least 97%, at least 99%, or 100% sequence identity to the sequence set forth in SEQ ID NO: 21.

23. The pharmaceutical formulation according to any one of the preceding claims, wherein:
a. said first antigen-binding region binding to human CD137 comprises
a first heavy chain variable region comprising the sequence set forth in SEQ ID NO: 15 or a sequence wherein up to 20 amino acid residues, such as up to 19, up to 18, up to 17, up to 16, up to 15, up to 14, up to 13, up to 12, up to 11, up to 10, up to 9, up to 8, up to 7, up to 6, up to 5, up to 4, up to 3, up to 2, up to 1 amino acid residues is/are modified as compared to the sequence set forth in SEQ ID NO: 15, the first heavy chain variable region (VH) comprising a CDR1, CDR2, and CDR3 sequence, as set forth in: SEQ ID NO: 9, 10 and 11, respectively; and
a first light chain variable region comprising the sequence set forth in SEQ ID NO: 16 or a sequence wherein up to 20 amino acid residues, such as up to 19, up to 18, up to 17, up to 16, up to 15, up to 14, up to 13, up to 12, up to 11, up to 10, up to 9, up to 8, up to 7, up to 6, up to 5, up to 4, up to 3, up to 2, up to 1 amino acid residues is/are modified as compared to the sequence set forth in SEQ ID NO: 16 the first light chain variable region (VL) comprising a CDR1, CDR2, and CDR3 sequence, as set forth in: SEQ ID NO: 13, GAS and SEQ ID NO: 14, respectively; and
b. said second antigen-binding region binding to human PD-L1 comprises
a second heavy chain variable region comprising the sequence set forth in SEQ ID NO: 17 or a sequence wherein up to 20 amino acid residues, such as up to 19, up to 18, up to 17, up to 16, up to 15, up to 14, up to 13, up to 12, up to 11, up to 10, up to 9, up to 8, up to 7, up to 6, up to 5, up to 4, up to 3, up to 2, up to 1 amino acid residues is/are modified as compared to the sequence set forth in SEQ ID NO: 17, the second heavy chain variable region (VH) comprising a CDR1, CDR2, and CDR3 sequence, as set forth in: SEQ ID NO: 18, 19 and 20, respectively; and
a second light chain variable region comprising the sequence set forth in SEQ ID NO: 21 or a sequence wherein up to 20 amino acid residues, such as up to 19, up to 18, up to 17, up to 16, up to 15, up to 14, up to 13, up to 12, up to 11, up to 10, up to 9, up to 8, up to 7, up to 6, up to 5, up to 4, up to 3, up to 2, up to 1 amino acid residues is/are modified as compared to the sequence set forth in SEQ ID NO: 21, the second light chain variable region (VH) comprising a CDR1, CDR2, and CDR3 sequence, as set forth in: SEQ ID NO: 22, DDN and SEQ ID NO: 23, respectively.

24. The pharmaceutical formulation according to any one of the preceding claims, wherein said binding agent comprises (i) a polypeptide comprising said first heavy chain variable region (VH) and further comprising a first heavy chain constant region (CH) and (ii) a polypeptide comprising said second heavy chain variable region (VH) and further comprising a second heavy chain constant region (CH).

25. The pharmaceutical composition according to any one of the preceding claims, which comprises (i) a polypeptide comprising said first light chain variable region (VL) and further comprising a first light chain constant region (CL) and (ii) a polypeptide comprising said second light chain variable region (VL) and further comprising a second light chain constant region (CL).

26. The pharmaceutical formulation according to any one of the preceding claims, which is an antibody comprising a first binding arm and a second binding arm, wherein
a. the first binding arm comprises i) a polypeptide comprising said first heavy chain variable region (VH) and said first heavy chain constant region (CH) and ii) a polypeptide comprising said first light chain variable region (VL) and said first light chain constant region (CL) and;
b. the second binding arm comprises i) a polypeptide comprising said second heavy chain variable region (VH) and said second heavy chain constant region (CH) and ii) a polypeptide comprising said second light chain variable region (VL) and said second light chain constant region (CL).

27. The pharmaceutical formulation according to any one of the preceding claims, wherein the first antigen-binding region binds to human CD137 as set forth in SEQ ID NO: 30, or a mature polypeptide thereof.

28. The pharmaceutical formulation according to any one of the preceding claims, wherein the first antigen-binding region binds to cynomolgus monkey (Macaca fascicularis) CD137, as set forth in SEQ ID NO: 31, or a mature polypeptide thereof.

29. The pharmaceutical formulation according to any one of the preceding claims, wherein the first antigen-binding region binds to human PD-L1 as set forth in SEQ ID NO: 28, or a mature polypeptide thereof.

30. The pharmaceutical formulation according to any one of the preceding claims, wherein the second antigen-binding region binds to cynomolgus monkey (Macaca fascicularis) PD-L1 as set forth in SEQ ID NO: 29, or a mature polypeptide thereof.

31. The pharmaceutical formulation according to any one of the preceding claims, wherein the second antigen-binding region inhibits the binding of human PD-L1 to human PD-1.

32. The pharmaceutical formulation according to any one of the preceding claims, wherein the binding agent is in the format of a full-length antibody or an antibody fragment.

33. The pharmaceutical formulation according to any one of the preceding claims, wherein the binding agent is of an isotype selected from the group consisting of IgG1, IgG2, IgG3, and IgG4.

34. The pharmaceutical formulation according to any one of the preceding claims, wherein the binding agent is a full-length IgG1 antibody.

35. The pharmaceutical formulation according to any one of the preceding claims, wherein
a. the first antigen-binding region binding to CD137 is derived from a chimeric antibody, and/or
b. the second antigen-binding region binding to human PD-L1 is derived from a chimeric antibody.

36. The pharmaceutical formulation according to any one of the preceding claims, wherein
a. the first antigen-binding region binding to CD137 is derived from a humanized antibody, and/or
b. the second antigen-binding region binding to human PD-L1 is derived from a humanized antibody.

37. The pharmaceutical formulation according to any one of the preceding claims, wherein
a. the first antigen-binding region binding to human CD137 is derived from a human antibody, and/or
b. the second antigen-binding region binding to human PD-L1 is derived from a human antibody.

38. The binding agent according to any one of the preceding claims, wherein
a. the first antigen-binding region binding to human CD137 is derived from a humanized antibody, and/or
b. the second antigen-binding region binding to human PD-L1 is derived from a human antibody.

39. The pharmaceutical formulation according to any one of claims 26 to 38, wherein each of the first and second heavy chain constant regions (CH) comprises one or more of a constant region domain 1 region (CH1 region), a hinge region, a CH2 region and a CH3 region, preferably at least a hinge region, a CH2 region and a CH3 region.

40. The pharmaceutical formulation according to claim 39, wherein each of the first and second heavy chain constant regions (CHs) comprises a CH3 region and wherein the two CH3 regions comprise asymmetrical mutations.

41. The pharmaceutical formulation according to any one of claims 25 to 40, wherein in said first heavy chain constant region (CH) at least one of the amino acids in a position corresponding to a position selected from the group consisting of T366, L368, K370, D399, F405, Y407, and K409 in a human IgG1 heavy chain according to EU numbering has been substituted, and in said second heavy chain constant region (CH) at least one of the amino acids in a position corresponding to a position selected from the group consisting of T366, L368, K370, D399, F405, Y407, and K409 in a human IgG1 heavy chain according to EU numbering has been substituted, and wherein said first and said second heavy chains are not substituted in the same positions.

42. The pharmaceutical formulation according to claim 41, wherein (i) the amino acid in the position corresponding to F405 in a human IgG1 heavy chain according to EU numbering is L in said first heavy chain constant region (CH), and the amino acid in the position corresponding to K409 in a human IgG1 heavy chain according to EU numbering is R in said second heavy chain constant region (CH), or (ii) the amino acid in the position corresponding to K409 in a human IgG1 heavy chain according to EU numbering is R in said first heavy chain, and the amino acid in the position corresponding to F405 in a human IgG1 heavy chain according to EU numbering is L in said second heavy chain.

43. The pharmaceutical formulation according to any of the preceding claims, wherein said antibody induces Fc-mediated effector function to a lesser extent compared to another antibody comprising the same first and second antigen binding regions and two heavy chain constant regions (CHs) comprising human IgG1 hinge, CH2 and CH3 regions.

44. The pharmaceutical formulation according to claim 43, wherein said first and second heavy chain constant regions (CHs) are modified so that the antibody induces Fc-mediated effector function to a lesser extent compared to an antibody which is identical except for comprising non-modified first and second heavy chain constant regions (CHs).

45. The pharmaceutical formulation according to any one of claims 43 to 44, wherein said Fc-mediated effector function is measured by binding to Fcγ receptors, binding to C1q, or induction of Fc-mediated cross-linking of Fcγ receptors.

46. The pharmaceutical formulation according to claim 45, wherein said Fc-mediated effector function is measured by binding to C1q.

47. The pharmaceutical formulation according to any one of claims 43-46, wherein said first and second heavy chain constant regions have been modified so that binding of C1q to said antibody is reduced compared to a wild-type antibody, preferably reduced by at least 70%, at least 80%, at least 90%, at least 95%, at least 97%, or 100%, wherein C1q binding is preferably determined by ELISA.

48. The pharmaceutical formulation according to any one of the preceding claims, wherein in at least one of said first and second heavy chain constant region (CH) one or more amino acids in the positions corresponding to positions L234, L235, D265, N297, and P331 in a human IgG1 heavy chain according to EU numbering, are not L, L, D, N, and P, respectively.

49. The pharmaceutical formulation according to claim 48, wherein the positions corresponding to positions L234 and L235 in a human IgG1 heavy chain according to EU numbering are F and E, respectively, in said first and second heavy chains.

50. The pharmaceutical formulation according to claim 48, wherein the positions corresponding to positions L234, L235, and D265 in a human IgG1 heavy chain according to EU numbering are F, E, and A, respectively, in said first and second heavy chain constant regions (HCs).

51. The pharmaceutical formulation according to claim 48, wherein the positions corresponding to positions L234, L235, and D265 in a human IgG1 heavy chain according to EU numbering of both the first and second heavy chain constant regions are F, E, and A, respectively, and wherein (i) the position corresponding to F405 in a human IgG1 heavy chain according to EU numbering of the first heavy chain constant region is L, and the position corresponding to K409 in a human IgG1 heavy chain according to EU numbering of the second heavy chain constant region is R, or (ii) the position corresponding to K409 in a human IgG1 heavy chain according to EU numbering of the first heavy chain is R, and the position corresponding to F405 in a human IgG1 heavy chain according to EU numbering of the second heavy chain is L.

52. The pharmaceutical formulation according to claim 48, wherein the positions corresponding to positions L234 and L235 in a human IgG1 heavy chain according to EU numbering of both the first and second heavy chain constant regions are F and E, respectively, and wherein (i) the position corresponding to F405 in a human IgG1 heavy chain according to EU numbering of the first heavy chain constant region is L, and the position corresponding to K409 in a human IgG1 heavy chain according to EU numbering of the second heavy chain is R, or (ii) the position corresponding to K409 in a human IgG1 heavy chain according to EU numbering of the first heavy chain constant region is R, and the position corresponding to F405 in a human IgG1 heavy chain according to EU numbering of the second heavy chain is L.

53. The pharmaceutical formulation according to any one of claims 26 to 52, wherein the first binding arm comprises a kappa (κ) light chain, such as a kappa light chain comprising the amino acid sequence set forth in SEQ ID NO: 26 and said second binding arm comprises a lambda (λ) light chain, such as a lambda light chain comprising the amino acid sequence set forth in SEQ ID NO: 27.

54. The pharmaceutical formulation according to any one of claims 26 to 52, wherein the first binding arm comprises a lambda (λ) light chain, such as a lambda light chain comprising the amino acid sequence set forth in SEQ ID NO: 27 and said second binding arm comprises a kappa (κ) light chain, such as a kappa light chain comprising the amino acid sequence set forth in SEQ ID NO: 26.

55. The pharmaceutical formulation according to any one of claims 26 to 52, wherein both the first binding arm and the second binding arm comprises a lambda (λ) light chain, such as a lambda light chain comprising the amino acid sequence set forth in SEQ ID NO: 27.

56. The pharmaceutical formulation according to any one of claims 26 to 52, wherein both the first binding arm and the second binding arm comprises a kappa (κ) light chain, such as a kappa light chain comprising the amino acid sequence set forth in SEQ ID NO: 26.

57. The pharmaceutical formulation according to claim any one of claims 26 to 56, wherein the first binding arm comprises the amino acid sequences set forth in SEQ ID NO: 24 and the second binding arm comprises the amino acid sequence set forth in SEQ ID NO: 25.

58. The pharmaceutical formulation according to claim any one of claims 26 to 56, wherein the first binding arm comprises the amino acid sequences set forth in SEQ ID NO: 25 and the second binding arm comprises the amino acid sequence set forth in SEQ ID NO: 24.

59. The pharmaceutical formulation according to any one of the preceding claims, wherein the binding agent induces and/or enhances proliferation of T cells.

60. The pharmaceutical formulation according to claim 59, wherein said T cells are CD4⁺ and/or CD8⁺ T cells.

61. The pharmaceutical formulation according to any one of the preceding claims, wherein the binding agent activates CD137 signaling only when the second antigen-binding region binds to PD-L1.

62. The pharmaceutical formulation according to claims 59 to 61, wherein proliferation of T cells is measured by co-culturing T-cells expressing a specific T-cell receptor (TCR) with dendritic cells (DCs) presenting the corresponding antigen on the major histocompatibility complex, which is recognized by the TCR.

63. The pharmaceutical formulation according to any one of the preceding claims, the formulation being an aqueous formulation.

64. A pharmaceutical formulation as defined in any one of the preceding claims for use as a medicament.

65. A pharmaceutical formulation as defined in any one of claims 1 to 64 for use in the treatment of cancer.

66. A method of treatment of a disease comprising administering an effective amount of a pharmaceutical formulation as defined in any one of claims 1 to 64 to a subject in need thereof.

67. The method according to claim 66, wherein the disease is cancer.

68. A method for producing a pharmaceutical formulation as defined in any one of claims 1 to 64, the method comprising providing a binding agent as defined in any one of claims 1 to 65 and combining it with:
a. a histidine buffer,
b. about 100 to about 400 mM of a sugar, and
c. about 0.001 to about 0.1% (w/v) non-ionic surfactant;
at a pH between about 4.5 and about 6.5.

69. A method of inducing cell death, or inhibiting growth and/or proliferation of a tumor cell expressing PD-L1 comprising administering an effective amount of a pharmaceutical formulation as defined in any one of claims 1 to 64 to a subject in need thereof and/or bearing said tumor cell.

70. The pharmaceutical formulation for use according to claim 65, or the method according to claim 67, wherein the cancer is characterized by the presence of solid tumors or is selected from the group consisting of: melanoma, ovarian cancer, lung cancer, colon cancer and head and neck cancer.

71. The pharmaceutical formulation for use according to claim 65, or the method according to claim 67 or 68, wherein the cancer is non-small cell lung cancer (NSCLC).

72. Use of a pharmaceutical formulation according to any one of claims 1 to 64, for the manufacture of a medicament, such as a medicament for the treatment of cancer, e.g. a cancer characterized by the presence of solid tumors or a cancer selected from the group consisting of: melanoma, ovarian cancer, lung cancer, colon cancer and head and neck cancer.

73. The use according to claim 72, wherein the lung cancer is non-small cell lung cancer (NSCLC).

74. The pharmaceutical formulation for use according to claim 64 or 65, the use according to any one of claims 72 to 73 or the method according to any one of claims 66, 67, 69, wherein the pharmaceutical formulation is administered intravenously.

75. The pharmaceutical formulation for use according to claim 64 or 65, the use according to any one of claims 72 to 73 or the method according to any one of claims 66, 67, 69, wherein the use or method comprises combination with one or more further therapeutic agents, such as a chemotherapeutic agent.


SEQUENCE LISTING (51 sequences)

SEQ ID NO: 1 - protein, 127 aa, Homo Sapiens
QVQLVQSGAEVKKPGASVKVSCQASGYRFSNFVIHWVRQAPGQRFEWMGWINPYNGNKEFSAKFQDRVTFTADTSANTAYMELRSLRSADTAVYYCARVGPYSWDDSPQDNYYMDVWGKGTTVIVSS

SEQ ID NO: 2 - protein, 8 aa, Homo Sapiens
GYRFSNFV

SEQ ID NO: 3 - protein, 8 aa, Homo Sapiens
INPYNGNK

SEQ ID NO: 4 - protein, 20 aa, Homo Sapiens
ARVGPYSWDDSPQDNYYMDV

SEQ ID NO: 5 - protein, 108 aa, Homo Sapiens
EIVLTQSPGTLSLSPGERATFSCRSSHSIRSRRVAWYQHKPGQAPRLVIHGVSNRASGISDRFSGSGSGTDFTLTITRVEPEDFALYYCQVYGASSYTFGQGTKLERK

SEQ ID NO: 6 - protein, 7 aa, Homo Sapiens
HSIRSRR

SEQ ID NO: 7 - protein, 9 aa, Homo Sapiens
QVYGASSYT

SEQ ID NO: 8 - protein, 114 aa, Homo Sapiens
QSLEESGGRLVTPGTPLTLTCTVSGFSLNDYWMSWVRQAPGKGLEWIGYIDVGGSLYYASWAKGRFTISRTSTTVDLKMTSLTTEDTATYFCARGGLTYGFDLWGPGTLVTVSS

SEQ ID NO: 9 - protein, 8 aa, Homo Sapiens
GFSLNDYW

SEQ ID NO: 10 - protein, 7 aa, Homo Sapiens
IDVGGSL

SEQ ID NO: 11 - protein, 11 aa, Homo Sapiens
ARGGLTYGFDL

SEQ ID NO: 12 - protein, 110 aa, Homo Sapiens
DIVMTQTPASVSEPVGGTVTINCQASEDISSYLAWYQQKPGQRPKRLIYGASDLASGVPSRFSASGSGTEYALTISDLESADAATYYCHYYATISGLGVAFGGGTEVVVK

SEQ ID NO: 13 - protein, 6 aa, Homo Sapiens
EDISSY

SEQ ID NO: 14 - protein, 12 aa, Homo Sapiens
HYYATISGLGVA

SEQ ID NO: 15 - protein, 117 aa, Homo Sapiens
EVQLVESGGGLVQPGRSLRLSCTASGFSLNDYWMSWVRQAPGKGLEWVGYIDVGGSLYYAASVKGRFTISRDDSKSIAYLQMNSLKTEDTAVYYCARGGLTYGFDLWGQGTLVTVSS

SEQ ID NO: 16 - protein, 110 aa, Homo Sapiens
DIVMTQSPSSLSASVGDRVTITCQASEDISSYLAWYQQKPGKAPKRLIYGASDLASGVPSRFSASGSGTDYTFTISSLQPEDIATYYCHYYATISGLGVAFGGGTKVEIK

SEQ ID NO: 17 - protein, 121 aa, Homo Sapiens
EVQLLEPGGGLVQPGGSLRLSCEASGSTFSTYAMSWVRQAPGKGLEWVSGFSGSGGFTFYADSVRGRFTISRDSSKNTLFLQMSSLRAEDTAVYYCAIPARGYNYGSFQHWGQGTLVTVSS

SEQ ID NO: 18 - protein, 8 aa, Homo Sapiens
GSTFSTYA

SEQ ID NO: 19 - protein, 8 aa, Homo Sapiens
FSGSGGFT

SEQ ID NO: 20 - protein, 14 aa, Homo Sapiens
AIPARGYNYGSFQH

SEQ ID NO: 21 - protein, 108 aa, Homo Sapiens
SYVLTQPPSVSVAPGQTARITCGGNNIGSKSVHWYQQKPGQAPVLVVYDDNDRPSGLPERFSGSNSGNTATLTISRVEAGDEADYYCQVWDSSSDHVVFGGGTKLTVL

SEQ ID NO: 22 - protein, 6 aa, Homo Sapiens
NIGSKS

SEQ ID NO: 23 - protein, 11 aa, Homo Sapiens
QVWDSSSDHVV

SEQ ID NO: 24 - protein, 330 aa, Artificial Sequence
  IgG1 with FEAR substitutions
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSRLTVDKSRWQQGNVFSCSVMHEALHNHYTQKSLSLSPGK

SEQ ID NO: 25 - protein, 330 aa, Artificial Sequence
  IgG1 with FEAL substitutions
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFLLYSKLTVDKSRWQQGNVFSCSVMHEALHNHYTQKSLSLSPGK

SEQ ID NO: 26 - protein, 107 aa, Homo Sapiens [sequence omitted]

SEQ ID NO: 27 - protein, 106 aa, Homo Sapiens
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 28 - protein, 290 aa, Homo Sapiens
  SIGNAL (1)..(18)
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 29 - protein, 290 aa, Macaca Fascicularis
  SIGNAL (1)..(18)
MRIFAVFIFTIYWHLLNAFTVTVPKDLYVVEYGSNMTIECKFPVEKQLDLTSLIVYWEMEDKNIIQFVHGEEDLKVQHSNYRQRAQLLKDQLSLGNAALRITDVKLQDAGVYRCMISYGGADYKRITVKVNAPYNKINQRILVVDPVTSEHELTCQAEGYPKAEVIWTSSDHQVLSGKTTTTNSKREEKLLNVTSTLRINTTANEIFYCIFRRLDPEENHTAELVIPELPLALPPNERTHLVILGAIFLLLGVALTFIFYLRKGRMMDMKKCGIRVTNSKKQRDTQLEET

SEQ ID NO: 30 - protein, 255 aa, Homo Sapiens
  SIGNAL (1)..(23)
MGNSCYNIVATLLLVLNFERTRSLQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQIISFFLALTSTALLFLLFFLTLRFSVVKRGRKKLLYIFKQPFMRPVQTTQEEDGCSCRFPEEEEGGCEL

SEQ ID NO: 31 - protein, 253 aa, Macaca Fascicularis
  SIGNAL (1)..(23)
MGNSCYNIVATLLLVLNFERTRSLQDLCSNCPAGTFCDNNRSQICSPCPPNSFSSAGGQRTCDICRQCKGVFKTRKECSSTSNAECDCISGYHCLGAECSMCEQDCKQGQELTKKGCKDCCFGTFNDQKRGICRPWTNCSLDGKSVLVNGTKERDVVCGPSPADLSPGASSATPPAPAREPGHSPQIIFFLALTSTVVLFLLFFLVLRFSVVKRSRKKLLYIFKQPFMRPVQTTQEEDGCSCRFPEEEEGGCE

SEQ ID NO: 32 - protein, 205 aa, Artificial Sequence
  Human-Wild boar chimeric CD137
  SIGNAL (1)..(23)
MGNSCYNIVATLLLVLNFERTRSVPDPCSNCSAGTFCGKNIQELCMPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQIISFFLALTSTALLGGCEL

SEQ ID NO: 33 - protein, 255 aa, Artificial Sequence
  Human-Wild boar chimeric CD137
  SIGNAL (1)..(23)
MGNSCYNIVATLLLVLNFERTRSLQDPCSNCPAGTFCDNNRNQICSPCPLNSFSSTGGQMNCDMCRKCEGVFKTKRACSPTRDAECECTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQIISFFLALTSTALLFLLFFLTLRFSVVKRGRKKLLYIFKQPFMRPVQTTQEEDGCSCRFPEEEEGGCEL

SEQ ID NO: 34 - protein, 255 aa, Artificial Sequence
  Human-Wild boar chimeric CD137
  SIGNAL (1)..(23)
MGNSCYNIVATLLLVLNFERTRSLQDPCSNCPAGTFCDNNRNQICSPCPPNSFSSAGGQRTCDICRQCKGVFRTRKECSSTSNAECDCVPGFRCLGAGCAMCEEYCQQGQELTQKGCKDCCFGTFNDQKRGICRPWTNCSLDGKSVLVNGTKERDVVCGPSPADLSPGASSVTPPAPAREPGHSPQIISFFLALTSTALLFLLFFLTLRFSVVKRGRKKLLYIFKQPFMRPVQTTQEEDGCSCRFPEEEEGGCEL

SEQ ID NO: 35 - protein, 255 aa, Artificial Sequence
  Human-Wild boar chimeric CD137
  SIGNAL (1)..(23)
MGNSCYNIVATLLLVLNFERTRSLQDPCSNCPAGTFCDNNRNQICSPCPPNSFSSAGGQRTCDICRQCKGVFRTRKECSSTSNAECDCTPGFHCLGAGCSMCEQDCKQGQELTKEGCKDCSFGTFNDEEHGVCRPWTDCSLDGKSVLVNGTKERDVVCGPSPADLSPGASSVTPPAPAREPGHSPQIISFFLALTSTALLFLLFFLTLRFSVVKRGRKKLLYIFKQPFMRPVQTTQEEDGCSCRFPEEEEGGCEL

SEQ ID NO: 36 - protein, 255 aa, Artificial Sequence
  Human-Wild boar chimeric CD137
  SIGNAL (1)..(23)
MGNSCYNIVATLLLVLNFERTRSLQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLAGKPVLMNGTKARDVVCGPRPADLSPGASSVTPPAPAREPGHSPQIISFFLALTSTALLFLLFFLTLRFSVVKRGRKKLLYIFKQPFMRPVQTTQEEDGCSCRFPEEEEGGCEL

SEQ ID NO: 37 - protein, 255 aa, Artificial Sequence
  Human-Wild boar chimeric CD137
  SIGNAL (1)..(23)
MGNSCYNIVATLLLVLNFERTRSLQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPTDFSPGTPSTTMPVPGGEPGHTSHIISFFLALTSTALLFLLFFLTLRFSVVKRGRKKLLYIFKQPFMRPVQTTQEEDGCSCRFPEEEEGGCEL

SEQ ID NO: 38 - protein, 255 aa, Sus Scrofa
  SIGNAL (1)..(23)
MGNGYYNIVATVLLVMNFERTRSVPDPCSNCSAGTFCGKNIQELCMPCPSNSFSSTSGQKACNVCRKCEGVFRTKKECSSTSNAVCECVPGFRCLGAGCAMCEEYCQQGQELTQEGCKDCSFGTFNDEEHGVCRPWTDCSLAGKPVLMNGTKARDVVCGPRPTDFSPGTPSTTMPVPGGEPGHTSHVIIFFLALMSTAVFVLVSYLALRFSVVQQGRKKLLYIVKQPFLKPAQTVQEEDACSCRFPEEEEGECEL

SEQ ID NO: 39 - protein, 255 aa, Loxodonta Africana
  SIGNAL (1)..(23)
MGNGYYNMVATVLLVMNFERTGAVQDSCRDCLAGTYCVKNESQICSPCPLNSFSSTGGQMNCDMCRKCEGVFKTKRACSPTRDAECECVSGFHCLGAGCTMCQQDCKQGQELTKEGCKDCCLGTFNDQKNGICRPWTNCSLEGKSVLANGTKKRDVVCGPPAADSFPDTSSVTVPAPERKPDHHPQIITFFLALISAALLFLVFFLVVRFSVAKWGRKKLLYIFKQPFIKPVQTAQEEDGCSCRFPEEEEGDCEL

SEQ ID NO: 40 - protein, 41 aa, Homo Sapiens
CPPNSFSSAGGQRTCDICRQCKGVFRTRKECSSTSNAECDC

SEQ ID NO: 41 - protein, 232 aa, Homo Sapiens
LQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQIISFFLALTSTALLFLLFFLTLRFSVVKRGRKKLLYIFKQPFMRPVQTTQEEDGCSCRFPEEEEGGCEL

SEQ ID NO: 42 - protein, 116 aa, Artificial Sequence
  VH-CD137 with FEAR substitutions
EVQLVQSGAEVKKPGESLRISCKGSGYSFSTYWISWVRQMPGKGLEWMGKIYPGDSYTNYSPSFQGQVTISADKSISTAYLQWSSLKASDTAMYYCARGYGIFDYWGQGTLVTVSS

SEQ ID NO: 43 - protein, 8 aa, Homo Sapiens
GYSFSTYW

SEQ ID NO: 44 - protein, 8 aa, Homo Sapiens
IYPGDSYT

SEQ ID NO: 45 - protein, 9 aa, Homo Sapiens
ARGYGIFDY

SEQ ID NO: 46 - protein, 108 aa, Homo Sapiens
SYELTQPPSVSVSPGQTASITCSGDNIGDQYAHWYQQKPGQSPVLVIYQDKNRPSGIPERFSGSNSGNTATLTISGTQAMDEADYYCATYTGFGSLAVFGGGTKLTVL

SEQ ID NO: 47 - protein, 6 aa, Homo Sapiens
NIGDQY

SEQ ID NO: 48 - protein, 11 aa, Homo Sapiens
ATYTGFGSLAV

SEQ ID NO: 49 - protein, 153 aa, Artificial sequence
  Recombinant human interleukin analog
  SIGNAL (1)..(20)
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMSEYADETATIVEFLNRWITFCQSIISTLT

SEQ ID NO: 50 - protein, 118 aa, Homo sapiens
EVQLVESGGGLVQPGGSLRLSCAASGFTFSDSWIHWYRQAPGKGLEWYAWISPYGGSTYYADSVKGRFTISADTSKNTAYLQMNSLRAEDTAVYYCARRHWPGGFDYWGQGTLVTVSS

SEQ ID NO: 51 - protein, 107 aa, Homo sapiens [sequence omitted]